(19) United States
(12) Patent Application Publication
(10) Pub. No.: US 2022/0241428 A1
(43) Pub. Date: Aug. 4, 2022

(54) MACROPHAGE SPECIFIC ENGAGER COMPOSITIONS AND METHODS OF USE THEREOF

(71) Applicant: Myeloid Therapeutics, Inc., Cambridge, MA (US)

(72) Inventors: Daniel GETTS, Stow, MA (US); Yuxiao Wang, Belmont, MA (US)

(21) Appl. No.: 17/618,349

(22) PCT Filed: Jun. 11, 2020
(86) PCT No.: PCT/US20/37312

§ 371 Date: Dec. 10, 2021

Related U.S. Application Data
(60) Provisional application No. 62/860,055, filed on Jun. 11, 2019.
(60) Provisional application No. 62/908,978, filed on Oct. 1, 2019.

(51) Int. Cl. A61K 47/68; A61K 47/65; A61K 47/54; A61K 47/55

(52) U.S. Cl. CPC A61K 47/6849; A61K 47/6815; A61K 47/55; A61K 47/65; A61K 47/549; A61K 47/6813

(57) ABSTRACT

The present disclosure provides compositions and methods for making and using therapeutic agents comprising myeloid cell specific engagers, used for immunotherapy of cancer or infection.

### CROSS REFERENCE

This application claims the benefit of U.S. Provisional Application No. 62/860,055, filed on Jun. 11, 2019, and U.S. Provisional Application No. 62/908,978, filed on Oct. 1, 2019, each of which is incorporated herein by reference in its entirety.

### BACKGROUND

Cellular immunotherapy is a promising new technology for fighting difficult to treat diseases, such as cancer, persistent infections and diseases that are refractory to other forms of treatment. Macrophages represent the dominant cell type present in a tumor or an infection site and possess several strategic advantages such that they can be potentially utilized to treat the disease most effectively. As natural sentinels of the immune system, these cells can sense and eliminate aberrant and non-healthy cell types, including cancer cells. However, potential use of macrophages for immunotherapy has not been fully explored. Newer avenues are sought for using these cell types towards development of improved therapeutics, including but not limited to T cell malignancies.

### SUMMARY

The present disclosure relates to new compositions and methods that initiate a target cell destruction pathway through phagocytosis. This application is based on an unexpected finding that when a phagocytic receptor is triggered with at least a second concurrent or subsequent activation signal in addition to binding to its classical ligand, the second or additional signal(s) can lead to efficient destruction of a target cell by phagocytosis. Presented herein are chimeric receptors, and chimeric receptor-binding extracellular elements designed for enhancing phagocytosis of a cell, such as a myeloid cell, or a monocyte or macrophage. Careful design and/or manipulation of the at least second concurrent or subsequent signal is useful for successful activation of a chimeric phagocytic receptor such as those described herein, such that the target cell is effectively destroyed thereafter. For example, the first signal (signal 1) can be mediated via phagocytosis/tethering receptors and the second signal (signal 2) can by mediated danger signals such as pathogen-associated molecular patterns (DAMPs), or cytokines that trigger nuclear factor-KB (NF-κB)-mediated upregulation of inflammatory genes. As described in the following section, triggering phagocytosis alone may be insufficient to activate monocytes or macrophages in the context of harnessing the phagocytic ability of monocytes or macrophages to kill cancer cells, and to drive an effective anti-tumor response.

One of the specific advantages of the inventions described here is that the compositions for effective cellular immunotherapy disclosed herein are cost-effective and efficient.

In some aspects, provided herein are new chimeric cell surface binding elements or “engagers” that bind to an extracellular portion of a chimeric phagocytic receptor, and bind additionally to at least a cell surface component on a target cell such as a cancer cell.

In one embodiment, the new chimeric engagers can bind to an extracellular portion of a chimeric phagocytic receptor, and additionally bind to one or more cell surface components, at least one of which is on a target cancer cell. Accordingly, an engager may be a bi-specific monocyte or macrophage engager (BiME) and have two binding portions, wherein one binding portion binds to an extracellular portion of a chimeric phagocytic receptor, and the other binds to the cell surface component on a target cell. Likewise, an engager may be a trispecific monocyte or macrophage engager (TriME) and have three binding portions, wherein one binding portion binds to an extracellular portion of a chimeric phagocytic receptor, another binding portion binds to the cell surface component on a target cell and the third binding portion binds to the cell surface component on the phagocytic cell.

In one aspect, the engager is a synthetic protein or a peptide, a conjugated protein or conjugated peptide. Provided herein is a composition comprising: a first therapeutic agent, wherein the therapeutic agent comprises: (a) a first binding domain, wherein the first binding domain is a first antibody or functional fragment thereof that specifically interacts with an antigen of a target cell, and (b) a second binding domain, wherein the second binding domain is a second antibody or functional fragment thereof that specifically interacts with a myeloid cell; wherein, (i) the first therapeutic agent is coupled to a first component, wherein the first component is an additional therapeutic agent or a third binding domain, or (ii) the composition comprises an additional therapeutic agent.

In one aspect, provided herein is a composition comprising: a therapeutic agent, wherein the therapeutic agent is an engager that comprises: (a) a first binding domain that specifically interacts with an antigen of a target cell, (b) a second binding domain that specifically interacts with a myeloid cell, and (c) a third binding domain that specifically interacts with the myeloid cell.

In one aspect, provided herein is a composition comprising: a therapeutic agent, wherein the therapeutic agent is an engager that comprises: (a) a first binding domain that specifically interacts with an antigen of a target cell, (b) a second binding domain, wherein the second binding domain: (i) specifically interacts with a myeloid cell (e.g., a monocyte or macrophage, or a dendritic cell) and promotes phagocytosis activity of the myeloid cell, or, (ii) specifically interacts with a myeloid cell and promotes inflammatory signaling of the myeloid cell, or (iii) specifically interacts with a myeloid cell or an adhesion molecule and promotes adhesion of the myeloid cell to the target cell, and (c) a third binding domain, wherein the third binding domain (i) specifically interacts with the myeloid cell and promotes phagocytic activity of the myeloid cell, or, (ii) specifically interacts with the myeloid cell and promotes inflammatory signaling of the myeloid cell, or, (iii) specifically interacts with the myeloid cell and promotes adhesion of the myeloid cell to the target cell, or, (iv) specifically interacts with the myeloid cell and inhibits anti-phagocytic activity of the myeloid cell mediated by the target cell, or (v) specifically interacts with the myeloid cell and inhibits anti-inflammatory activity of the myeloid cell mediated by the target cell.

In some embodiments, the myeloid cell is a monocyte or macrophage cell.

In some embodiments, the target cell is a cancer cell.

In some embodiments, the second binding domain that specifically interacts with a myeloid cell interacts with a phagocytic or tethering receptor of the myeloid cell or monocyte or macrophage cell.

In some embodiments, the third binding domain that specifically interacts with a myeloid cell interacts with an extracellular region of a first phagocytic or tethering receptor of the myeloid cell or monocyte or macrophage cell.

The composition of any one of the preceding claims wherein any one of binding domains of the therapeutic agent comprises the binding domain of an antibody, a functional fragment of an antibody, a variable domain thereof, a V_{H} domain, a V_{L} domain, a VNAR domain, a V_{HH} domain, a single chain variable fragment (scFv), an Fab, a single-domain antibody (sdAb), a nanobody, a bispecific antibody, a diabody, or a functional fragment or a combination thereof.

In some embodiments, the therapeutic agent is a recombinant protein or more than one recombinant proteins.

In some embodiments, the therapeutic agent comprises recombinant proteins comprising one or more fusion proteins.

In some embodiments, the therapeutic agent is a recombinant protein comprising an antibody, a functional fragment of an antibody, a variable domain thereof, a V_{H} domain, a V_{L} domain, a VNAR domain, a V_{HH} domain, a single chain variable fragment (scFv), an Fab, a single-domain antibody (sdAb), a nanobody, a bispecific antibody, a diabody, or a functional fragment or a combination thereof. In some embodiments, the therapeutic agent is a recombinant protein or more than one recombinant proteins, each comprising multiple binding fragments, each binding fragment constituting a functional fragment of an antibody, a variable domain thereof, a V_{H} domain, a V_{L} domain, a VNAR domain, a V_{HH} domain, a single chain variable fragment (scFv), an Fab, a single-domain antibody (sdAb), a nanobody, a bispecific antibody, a diabody, or a functional fragment or a combination thereof.

In some embodiments, the therapeutic agent is a recombinant protein (the engager) comprising multiple binding domains, each having individual binding specificities, that are each linked together by linkers (e.g., peptide linkers) that exhibit complementary binding with each other. For example, one binding domain of the recombinant protein is fused with the first of a pair of linker peptides, and the other binding domain is fused with the second of the pair of linker peptides, wherein, the pair of linker peptides exhibit complementary binding with each other, wherein the pair of linker peptides comprise: (a) leucine zipper domains that exhibit complementary binding with each other; for example, leucine zippers in naturally occurring protein-protein interactions, such as the zipper sequences within the binding regions of c-Fos and c-Jun proteins, (b) synthetic peptides designed to specifically bind to each other via designed affinities, such as synthetic clasps.

In some embodiments, the therapeutic agent is a recombinant protein comprising multiple binding fragments configured to facilitate accelerated association with each other by means of leucine zipper peptide pairs comprised in the recombinant proteins.

In some embodiments, the therapeutic agent is a recombinant protein comprising multiple binding fragments configured to facilitate accelerated association with each other by means of c-Fos/c-Jun binding domains in the peptide pairs comprised within the recombinant proteins.

In some embodiments, the therapeutic agent is a recombinant protein comprising multiple binding fragments configured to facilitate accelerated association with each other by means of synthetic clasps.

In some embodiments, the antigen on the target cell to which the first binding domain binds, is a cancer antigen or a pathogenic antigen on the target cell or an autoimmune antigen.

In some embodiments, the antigen on the target cell to which the first binding domain binds, is a viral antigen

In some embodiments, the antigen on the target cell to which the first binding domain binds is a T-lymphocyte antigen.

In some embodiments, the antigen on the target cell to which the first binding domain binds is an extracellular antigen.

In some embodiments, the antigen on the target cell to which the first binding domain binds is an intracellular antigen.

In some embodiments, the antigen on the target cell to which the first binding domain binds is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor vIII (EGFRvIII), Mesothelin, Human Epidermal Growth Factor Receptor 2 (HER2), Mesothelin, EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFR-beta, SSEA-4, EGFR, NCAM, prostase, PAP, ELF2M, GM3, TEM7R, CLDN6, TSHR, GPRC5D, ALK, IGLL1 and combinations thereof.

In some embodiments, the antigen on the target cell to which the first binding domain binds is selected from the group consisting of CD2, CD3, CD4, CD5, CD7, CCR4, CD8, CD30, CD45, CD56.

In some embodiments, the antigen on the target cell to which the first binding domain binds is an ovarian cancer antigen or a T lymphoma antigen.

In some embodiments, the antigen on the target cell to which the first binding domain binds is an integrin receptor.

In some embodiments, the second binding domain or the third binding domain binds to an integrin receptor.

In some embodiments, the second binding domain or the third binding domain binds to an integrin receptor selected from the group consisting of α1, α2, αIIb, α3, α4, α5, α6, α7, α8, α9, α10, α11, αD, αE, αL, αM, αV, αX, β1, β2, β3, β4, β5, β6, β7, and β8.

In some embodiments, the therapeutic agent binds to a phagocytic or tethering receptor that comprises a phagocytosis activation domain.

In some embodiments, the therapeutic agent binds to a receptor or a protein selected from the group consisting the receptors listed in Table 2A and Table 2B, or a fragment thereof.

In some embodiments, the therapeutic agent binds to a phagocytic receptor selected from the group consisting of lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor I, CR1, CD35, CR3, CR4, Tim-1, Tim-4 and CD169.

In some embodiments, the therapeutic agent binds to a receptor comprising an intracellular signaling domain that comprises a pro-inflammatory signaling domain.

In some embodiments, the first therapeutic agent comprises a polypeptide that is less than 1000 amino acids or 1000 nm in length or 1000 nm.

In some embodiments, the first therapeutic agent comprises a polypeptide that is less than 500 amino acids or 500 nm in length.

In some embodiments, the first therapeutic agent comprises a polypeptide that is 200-1000 amino acids or 200-1000 nm in length.

In some embodiments, engagement of the binding domains of the first therapeutic agent contacts the cancer cell to the myeloid cell.

In some embodiments, the second binding domain specifically interacts with a myeloid cell and promotes phagocytosis activity of the myeloid cell.

In some embodiments, the second binding domain specifically interacts with a myeloid cell and promotes inflammatory signaling of the myeloid cell.

In some embodiments, the second binding domain specifically interacts with a myeloid cell or an adhesion molecule and promotes adhesion of the myeloid cell to the target cell.

In some embodiments, the second binding domain specifically interacts with a myeloid cell and inhibits anti-phagocytic activity of the myeloid cell mediated by the target cell.

In some embodiments, the second binding domain specifically interacts with a myeloid cell and inhibits anti-inflammatory activity of the myeloid cell mediated by the target cell.

In some embodiments, the second and/or the third binding domain promotes phagocytic activity of the myeloid cell.

In some embodiments, the second and/or the third binding domain promotes inflammatory signaling of the myeloid cell.

In some embodiments, the second and/or the third binding domain specifically interacts with a myeloid cell or an adhesion molecule and promotes adhesion of the myeloid cell to the target cell.

In some embodiments, the second and/or the third binding domain inhibits anti-phagocytic activity of the myeloid cell mediated by the target cell.

In some embodiments, the second and/or the third binding domain inhibits anti-inflammatory activity of the myeloid cell mediated by the target cell.

In some embodiments, the therapeutic agent comprises a therapeutic polypeptide.

In some embodiments, the therapeutic agent comprises a recombinant nucleic acid encoding the therapeutic polypeptide.

In some embodiments, the third binding domain or the additional therapeutic agent comprises a CD47 antagonist, a CD47 blocker, an antibody, a chimeric CD47 receptor, a sialidase, a cytokine, a proinflammatory gene, a procaspase, or an anti-cancer agent.

In some embodiments, the first binding domain, the second binding domain and the third binding domain bind to distinct non-identical target antigens.

In some embodiments, the first binding domain, the second binding domain or the third binding domain is a ligand binding domain.

In some embodiments, the first, the second or the third binding domains are operably linked by one or more linkers.

In some embodiments, the linker is a polypeptide. In some embodiments, the linker is a functional peptide. In some embodiments, the linker is a ligand for a receptor. In some embodiments, the linker is a ligand for a monocyte or macrophage receptor. In some embodiments, the linker activates the receptor. In some embodiments, the linker inhibits the receptor. In some embodiments, the linker is a ligand for a M2 monocyte or macrophage. In some embodiments, the linker is a ligand for a TLR receptor. In some embodiments, the linker activates the TLR receptor.

In some embodiments, the first, the second and/or the third binding domains are associated with a mask that binds to the binding domain.

In some embodiments, the mask is an inhibitor that inhibits the interaction of binding domain to its target when the mask remains associated with the respective binding domain.

In some embodiments, the mask is associated with the binding domain via a peptide linker.

In some embodiments, the peptide linker comprises a cleavable moiety.

In some embodiments, the cleavable moiety is cleaved by a protein or an enzyme selectively abundant in the site of the cancer or tumor.

In some embodiments, the third binding domain that specifically interacts with an extracellular region of a second receptor of the monocyte or macrophage activates the monocyte or macrophage.

In some embodiments, upon binding of the therapeutic agent to the myeloid cell, the killing or phagocytosis activity of the myeloid cell is increased by at least 10%, or 20%, or 30%, or 40%, or 50%, or 60%, or 70% or 90% or 100% compared to a myeloid cell not bound by the therapeutic agent, as measured by a particle uptake assay.

In some embodiments, engagement of the binding domains of first therapeutic agent triggers phagocytosis of the cancer cell by the myeloid cell.

In some embodiments, engagement of the second therapeutic agent potentiates or increases the phagocytic killing of the cancer cell by the myeloid cell.

In some embodiments, the second or third binding domain binds to an extracellular of IgA, IgD, IgE, IgG, IgM, FcγRI, FcγRIIA, FcγRIIB, FcγRIIC, FcγRIIIA, FcγRIIIB, FcRn, TRIM21, FcRL5.

In some embodiments, the second or the third binding domain comprises an M2 domain.

In some embodiments, the second or the third binding domain comprises a LIGHT domain or an HVEM binding domain.

In some embodiments, the second or the third binding domain comprises a HVEM binding domain.

In some embodiments, the second or the third binding domain comprises a GITR binding domain.

In some embodiments, the first binding domain comprises a sequence having an amino acid sequence with at least 80%, 85%, 90%, 95% or 100% sequence identity to a sequence selected from the group consisting of SEQ ID NOs: 27, 28, 111, 112, 113, 115, 143 and 144.

In some embodiments, the second binding domain comprises a sequence having an amino acid sequence with at least 80%, 85%, 90%, 95% or 100% sequence identity to a sequence selected from the group 141 and 142.

In some embodiments, the first component comprises an amino acid sequence GGQEINSSYGG (SEQ ID NO: 105) or QEINSSY (SEQ ID NO: 129).

In some embodiments, the first component comprises an amino acid sequence GGAPPHALSGG (SEQ ID NO: 109) or APPHALS (SEQ ID NO: 137).

In some embodiments, the linker comprises an amino acid sequence GGQEINSSYGG (SEQ ID NO: 105), or QEINSSY (SEQ ID NO: 129) or GGAPPHALSGG (SEQ ID NO: 109) or APPHALS (SEQ ID NO: 137).

In some embodiments, provided herein is a bispecific or trispecific engager, comprising a sequence having an amino acid sequence with at least 80%, 85%, 90%, 95% or 100% sequence identity to SEQ ID NO: 151.

In some embodiments, provided herein is a bispecific or trispecific engager, comprising a sequence having an amino acid sequence with at least 80%, 85%, 90%, 95% or 100% sequence identity to SEQ ID NO: 152.

Provided herein is a pharmaceutical composition comprising: a first therapeutic agent, wherein the therapeutic agent comprises one or more polypeptides or recombinant nucleic acids encoding the one or more polypeptides, wherein the one or more polypeptides comprise: a first binding domain, wherein the first binding domain is a first antibody or functional fragment thereof that specifically interacts with an antigen of a target cell, and a second binding domain, wherein the second binding domain is a second antibody or functional fragment thereof that specifically interacts with a myeloid cell; wherein, (i) the first therapeutic agent is coupled to a first component, wherein the first component is an additional therapeutic agent or a third binding domain, or (ii) the composition comprises an additional therapeutic agent; and an acceptable pharmaceutical salt or excipient.

In some embodiments, the first therapeutic agent of the pharmaceutical composition comprises a single polypeptide. In some embodiments, the first therapeutic agent of the pharmaceutical composition comprises multiple polypeptides. In some embodiments, the first therapeutic agent of the pharmaceutical composition is a recombinant nucleic acid encoding the one or more polypeptides. In some embodiments, the pharmaceutical composition further comprises a second therapeutic agent.

Provided herein is a method of treatment, comprising: administering to the subject in need thereof, a pharmaceutical composition, comprising: a first therapeutic agent, wherein the therapeutic agent comprises one or more polypeptides or recombinant nucleic acids encoding the one or more polypeptides, wherein the one or more polypeptides comprise: a first binding domain, wherein the first binding domain is a first antibody or functional fragment thereof that specifically interacts with an antigen of a target cell, and a second binding domain, wherein the second binding domain is a second antibody or functional fragment thereof that specifically interacts with a myeloid cell; wherein, (i) the first therapeutic agent is coupled to a first component, wherein the first component is an additional therapeutic agent or a third binding domain, or (ii) the composition comprises an additional therapeutic agent; and an acceptable pharmaceutical salt or excipient.

In some embodiments, the method of treatment further comprises administering a second therapeutic agent. In some embodiments, the method of treatment further comprises administering the pharmaceutical composition comprises administering the pharmaceutical composition intravenously.

In some embodiments, the method of treatment further comprises the administering the pharmaceutical composition comprises administering the pharmaceutical composition subcutaneously. In some embodiments, the method of treatment further comprises administering the pharmaceutical composition comprises injecting the pharmaceutical composition.

In some embodiments, the target cell is a cancer cell.

In some embodiments, the target cell is a cancer cell that is a lymphocyte.

In some embodiments, the target cell is a cancer cell that is an ovarian cancer cell.

In some embodiments, the target cell is a cancer cell that is an ovarian pancreatic cell.

In some embodiments, the target cell is a cancer cell that is a glioblastoma cell.

In some embodiments, the recombinant nucleic acid is DNA.

In some embodiments, the recombinant nucleic acid is RNA.

In some embodiments, the recombinant nucleic acid is mRNA.

In some embodiments, the recombinant nucleic acid is a circRNA.

In some embodiments, the recombinant nucleic acid is a tRNA.

In some embodiments, the recombinant nucleic acid is a microRNA.

Provided herein is a vector, comprising the composition described above.

In some embodiments, vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions.

Provided herein is a polypeptide encoded by the recombinant nucleic acid of the composition described above.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” herein), of which:

FIG. 1A is a graphical representation of the exemplary extracellular stimulants, receptors and pathways generating a dual signal for a myeloid cell: a signal 1 and a signal 2.

FIG. 1B is a graphical representation of a simplified engager construct with a binder A, a liner L and a second binder B. In this simplified diagram, binder A binds to a cell surface biomolecule on a target cell; binder B binds to a cell surface biomolecule on a myeloid cell.

FIG. 2A is a graphical representation of bispecific scFv engager with protease cleavable masked site, that are the antigen binding domains.

FIG. 2B is a graphical representation of bispecific V_{HH} engager with protease cleavable masked antigen binding domains.

FIG. 3A is a graphical representation of an exemplary bispecific scFv engager with protease cleavable masked site, and a peptide linker connecting the two scFv engagers that are the antigen binding domains; in this case, the peptide linker is an additional therapeutic agent or a third binding domain, specifically a TLR4 ligand peptide.

FIG. 3B is a graphical representation of an exemplary bispecific V_{HH} engager with protease cleavable masked antigen binding domains, and a peptide linker connecting the two protease cleavable masked antigen binding domains; in this case, the peptide linker is an additional therapeutic agent or a third binding domain, specifically a TLR4 ligand peptide.

FIG. 3C is a graphical representation of an exemplary bispecific scFv engager with protease cleavable masked site, and a peptide linker connecting the two scFv engagers that are the antigen binding domains; in this case, the peptide linker is an additional therapeutic agent or a third binding domain, specifically a M2 targeting peptide.

FIG. 3D is a graphical representation of an exemplary bispecific V_{HH} engager with protease cleavable masked antigen binding domains, and a peptide linker connecting the two protease cleavable masked antigen binding domains; in this case, the peptide linker is an additional therapeutic agent or a third binding domain, specifically a M2 targeting peptide.

FIG. 3E depicts data indicating cytokine production by monocytes cultured overnight in the presence of each TLR peptide indicated.

FIG. 3F shows a graphical illustration of the protein structure of a bispecific binder construct CD5-RS01-CD16, having two scFv binders specific for CD5 and CD16 respectively, and a TLR4 synthetic peptide linker (RS01).

FIG. 3G shows expression data of the CD5-RS01-CD16 demonstrated in FIG. 3F. Lanes M1 and M2, Commercially available protein molecular weight marker from TaKaRa, Cat No. 3452 and GenScript, Cat No. M00521 respectively. Lanes 1 and 2 are SDS PAGE results or western blot results as indicated, under reducing and non-reducing conditions respectively. Lane P, positive control (Multiple tag, Gene Script, Cat No. M0101). Primary Antibody used for Western Blot: Mouse anti-His mAb (GenScripts, Cat. No. A00186).

FIG. 3H shows a graphical illustration of a protein structure of bispecific binder construct CD5-RSO9-CD16, having two scFv binders specific for CD5 and CD16 respectively, and a TLR4 synthetic peptide linker (RS09).

FIG. 3I depicts expression data of the CD5-RSO9-CD16 demonstrated in FIG. 3H. Lane annotation and indices are as indicated in description for FIG. 3G.

FIG. 4A is a graphical representation of an exemplary trispecific scFv engager.

FIG. 4B is a graphical representation of an exemplary trispecific V_{HH} engager.

FIG. 4C is a graphical representation of an exemplary mode of action of a trispecific engager.

FIG. 5Ai depicts a graphical representation of the structural configuration of a recombinant bispecific scFv engager, where each of the binding domains is masked by an agent (a mask), that prevents interaction of the binding domain with its cognate substrate. The mask is attached with the terminal section of each light chain by a cleavable linker, in the example a metalloprotease (MMP2) substrate. Arrows point to the structural components of the recombinant bispecific scFv engager, which are as follows: 1, mask; 2, MMP2 substrate linker; 3, ABD1 (antigen binding domain 1)-light chain; 3′, ABD2 (antigen binding domain 2)-light chain; 4, a linker connecting the binding domain light chain and the binding domain heavy chain; 5, ABD1 (antigen binding domain 1) heavy chain; 5′, ABD2 (antigen binding domain 2) heavy chain.

FIG. 5Aii depicts a graphical representation of the structural configuration of a recombinant bispecific diabody engager, where each of the binding domains is masked by an agent (a mask), that prevents interaction of the binding domain with its cognate substrate. The mask is attached with the terminal section of each light chain by a cleavable linker, in the example a metalloprotease (MMP2) substrate. Arrows point to the structural components of the recombinant bispecific diabody engager, which are as follows: 1, mask; 2, MMP2 substrate linker; 3, ABD1 (antigen binding domain 1)-light chain; 3′, ABD2 (antigen binding domain 2)-light chain; 4, linker connecting the ABD1 light chain and the ABD1 heavy chain; 4′, linker connecting the ABD2 light chain and the ABD2 heavy chain; 5, ABD2 heavy chain; 5′, ABD1 heavy chain.

FIG. 5B depicts a graphical representation of the linear construct for a single chain of the bispecific scFv. The parts are corresponding to FIG. 5Ai or FIG. 5Aii are depicted within the linearized diagram from N-terminal to C-terminal.

FIG. 6 depicts exemplary modular constructs comprising two or three binding domains to utilize as bispecific and trispecific engagers.

FIG. 7A upper panel is a graphical representation of MD2 mediated dimerization of TLR4 receptor, which leads to TLR activation. FIG. 7A lower panel is an exemplary design of a monocyte or macrophage specific engager, where one binding domain can bind to a tumor cell associated molecule (tumor antigen), another binding domain can bind to a monocyte or macrophage receptor, in this case an FcR. The third domain is an MD2 domain, which can bind to and dimerize TLR4 receptors, to activate them.

FIG. 7B is a graphical representation that shows the mode of action of the monocyte or macrophage specific engager of FIG. 7A.

FIG. 8A is an exemplary design of a monocyte or macrophage specific engager, where one binding domain can bind to a tumor cell associated molecule (tumor antigen), a second binding domain can bind to a monocyte or macrophage receptor, in this case an FcR. The third domain is an LIGHT domain, which can engage with monocyte or macrophage HVEM and activate an inflammatory signal in the monocyte or macrophage.

FIG. 8B is a graphical representation that shows the mode of action of the monocyte or macrophage specific engager of FIG. 8A.

FIG. 9A is an exemplary design of a monocyte or macrophage specific engager, where one binding domain can bind to a tumor cell associated molecule (tumor antigen), and a second binding domain can bind to a monocyte or macrophage receptor, in this case an FcR. The third domain is a GIRT ligand (GIRTL) domain or alternatively an antigen binding domain of anti-GITR antibody that can activate monocyte or macrophage receptor GITR, and can induce an inflammatory signal in the monocyte or macrophage.

FIG. 9B is a graphical representation that shows the mode of action of the monocyte or macrophage specific engager of FIG. 9A.

FIG. 10A shows exemplary heterodimeric antibody-based engager molecule designs, comprising peptides with leucine zipper domains. L1, L2 indicate ligands.

FIG. 10B shows exemplary heteromultimeric antibody-based engager molecule designs, comprising peptides with leucine zipper domains. L1-L4 indicate ligands.

FIG. 10C shows exemplary heterodimeric antibody-based engager molecule designs, comprising peptides having synthetic anchoring design. L1, L2 indicate ligands, and ‘m’ and ‘n’ indicate synthetic binding designs.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

An “agent” can include any type of molecule and includes, but is not limited to, an antibody, a peptide, a protein, a polynucleotide (e.g., an oligonucleotide, RNA, or DNA), a small molecule, derivatives thereof and analogs thereof.

A “biologic sample” is any tissue, cell, fluid, or other material derived from an organism. As used herein, the term “sample” includes a biologic sample such as any tissue, cell, fluid, or other material derived from an organism.

“Specifically binds” refers to a condition in which a compound (e.g., peptide) recognizes and binds to a molecule (e.g., peptide or polypeptide), but does not substantially recognize and bind other molecules in a sample, for example, a biological sample, that is, the compound exhibits a selective binding to a molecule. A “binder” as described herein includes, but is not limited to, a protein, a polypeptide or fragments thereof, that exhibits specific binding to a cognate molecule. A binder may refer to an antigen binding domain, such as the first binding domain of a bispecific or trispecific engager, or the second antigen binding domain of a bispecific or trispecific engager, and so on. In some cases, a binder may be any biomolecule or fragment thereof, such as a peptide or conjugated peptide or a ligand that can specifically bind to a receptor on a cell and therefore exhibits specific binding of one portion of an exemplary engager.

The term “immune response” includes T cell mediated and/or B cell mediated immune responses that are influenced by modulation of T cell costimulation. Exemplary immune responses include T cell responses, e.g., cytokine production, and cellular cytotoxicity. In addition, the term immune response includes immune responses that are indirectly affected by T cell activation, e.g., antibody production (humoral responses) and activation of cytokine responsive cells, e.g., monocytes or macrophages.

A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. The term “derivative” encompasses both amino acid sequence variants of polypeptide and covalent modifications thereof.

The terms “phagocytic cells” and “phagocytes” are used interchangeably herein to refer to a cell that is capable of phagocytosis. There are three main categories of phagocytes: macrophages, mononuclear cells (histiocytes and monocytes); polymorphonuclear leukocytes (neutrophils) and dendritic cells.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

As used herein, the term “antigen-presenting cell” or “antigen-presenting cells” or its abbreviation “APC” or “APCs” refers to a cell or cells capable of endocytosis adsorption, processing and presenting of an antigen. The term includes professional antigen presenting cells for example; B lymphocytes, monocytes, dendritic cells (DCs) and Langerhans cells, as well as other antigen presenting cells such as keratinocytes, endothelial cells, glial cells, fibroblasts and oligodendrocytes. The term “antigen presenting” means the display of antigen as peptide fragments bound to MHC molecules, on the cell surface. Many different kinds of cells may function as APCs including, for example, monocytes or macrophages, B cells, follicular dendritic cells and dendritic cells. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

An “epitope” refers to a portion of an antigen or other macromolecule capable of forming a binding interaction with the variable region binding pocket of an antibody or TCR. The term includes any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three dimensional structural characteristics as well as specific charge characteristics.

In some embodiments, the phagocytic receptor fusion protein (PFP) comprises an extracellular antigen binding domain specific to an antigen of a target cell, fused to the phagocytic receptor. A target cell is, for example, a cancer cell. In some embodiments, the phagocytic cell, after engulfment of the cancer cell may present the cancer antigen on its cell surface to activate a T cell.

As used herein the term “antigen” is any organic or inorganic molecule capable of stimulating an immune response. The term “antigen” as used herein extends to any molecule such as, but not limited, to a peptide, polypeptide, protein, nucleic acid molecule, carbohydrate molecule, organic or inorganic molecule capable of stimulating an immune response.

In some embodiments, the phagocytic receptor fusion protein may comprise an extracellular domain, which comprises an antibody domain or a antigen binding portion thereof that can bind to a cancer antigen or a cell surface molecule on a cancer cell. The term “antibody” or “antibody moiety” is includes, but is not limited to any polypeptide chain-containing molecular structure that recognizes an epitope. Antibodies utilized in the present invention may be polyclonal antibodies, although monoclonal antibodies are preferred because they may be reproduced by cell culture or recombinantly, and can be modified to reduce their antigenicity. The term includes IgG (including IgG1, IgG2, IgG3, and IgG4), IgA (including IgA1 and IgA2), IgD, IgE, IgM, and IgY, and is meant to include whole antibodies, including single-chain whole antibodies, and antigen-binding (Fab) fragments thereof. Antigen-binding antibody fragments include, but are not limited to, Fab, Fab′ and F(ab′)2, Fd (consisting of V_{H} and CH1), single-chain variable fragment (scFv), single-chain antibodies, disulfide-linked variable fragment (dsFv) and fragments comprising either a V_{L} or V_{H} domain. The antibodies can be from any animal origin. Antigen-binding antibody fragments, including single-chain antibodies, can comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex. A person of skill in the art will recognize that a variety of immunoaffinity techniques are suitable to enrich soluble proteins, such as soluble HLA-peptide complexes or membrane bound HLA-associated polypeptides, e.g., which have been proteolytically cleaved from the membrane. These include techniques in which (1) one or more antibodies capable of specifically binding to the soluble protein are immobilized to a fixed or mobile substrate (e.g., plastic wells or resin, latex or paramagnetic beads), and (2) a solution containing the soluble protein from a biological sample is passed over the antibody coated substrate, allowing the soluble protein to bind to the antibodies. The substrate with the antibody and bound soluble protein is separated from the solution, and optionally the antibody and soluble protein are disassociated, for example by varying the pH and/or the ionic strength and/or ionic composition of the solution bathing the antibodies. Alternatively, immunoprecipitation techniques in which the antibody and soluble protein are combined and allowed to form macromolecular aggregates can be used. The macromolecular aggregates can be separated from the solution by size exclusion techniques or by centrifugation.

The adaptive immune system reacts to molecular structures, referred to as antigens, of the intruding organism. Unlike the innate immune system, the adaptive immune system is highly specific to a pathogen. Adaptive immunity can also provide long-lasting protection; for example, someone who recovers from measles is now protected against measles for their lifetime. There are two types of adaptive immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in T cells, of the body. Cytotoxic T cells recognize these antigens and kill the cells that harbor the antigens.

The term “major histocompatibility complex (MHC)”, “MHC molecules”, or “MHC proteins” refers to proteins capable of binding antigenic peptides resulting from the proteolytic cleavage of protein antigens inside phagocytes or antigen presenting cells and for the purpose of presentation to and activation of T lymphocytes. Such antigenic peptides represent T cell epitopes. The human MHC is also called the HLA complex. Thus, the term “human leukocyte antigen (HLA) system”, “HLA molecules” or “HLA proteins” refers to a gene complex encoding the MHC proteins in humans. The term MHC is referred as the “H-2” complex in murine species. Those of ordinary skill in the art will recognize that the terms “major histocompatibility complex (MHC)”, “MHC molecules”, “MHC proteins” and “human leukocyte antigen (HLA) system”, “HLA molecules”, “HLA proteins” are used interchangeably herein.

HLA proteins are ty[ically classified into two types, referred to as HLA class I and HLA class II. The structures of the proteins of the two HLA classes are very similar; however, they can have different functions. Class I HLA proteins are present on the surface of almost all cells of the body, including most tumor cells. Class I HLA proteins are loaded with antigens that usually originate from endogenous proteins or from pathogens present inside cells, and are then presented to naïve or cytotoxic T-lymphocytes (CTLs). HLA class II proteins are present on antigen presenting cells (APCs), including but not limited to dendritic cells, B cells, and monocytes or macrophages. They mainly present peptides, which are processed from external antigen sources, e.g. outside of the cells, to helper T cells. Most of the peptides bound by the HLA class I proteins originate from cytoplasmic proteins produced in the healthy host cells of an organism itself, and do not normally stimulate an immune reaction.

In HLA class II system, phagocytes such as monocytes or macrophages and immature dendritic cells take up entities by phagocytosis into phagosomes—though B cells exhibit the more general endocytosis into endosomes—which fuse with lysosomes whose acidic enzymes cleave the uptaken protein into many different peptides. Autophagy is a source of HLA class II peptides. Via physicochemical dynamics in molecular interaction with the HLA class II variants borne by the host, encoded in the host's genome, a particular peptide exhibits immunodominance and loads onto HLA class II molecules. These are trafficked to and externalized on the cell surface. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells is required for immune responses to foreign antigens. Once activated, CD4+ T cells promote B cell differentiation and antibody production, as well as CD8+ T cell (CTL) responses. CD4+ T cells also secrete cytokines and chemokines that activate and induce differentiation of other immune cells. HLA class II molecules are heterodimers of α- and β-chains that interact to form a peptide-binding groove that is more open than class I peptide-binding grooves. Peptides bound to HLA class II molecules are believed to have a 9-amino acid binding core with flanking residues on either N- or C-terminal side that overhang from the groove. These peptides are usually 12-16 amino acids in length and often contain 3-4 anchor residues at positions P1, P4, P6/7 and P9 of the binding register (Rossjohn et al., 2015).

HLA alleles are expressed in codominant fashion, meaning that the alleles (variants) inherited from both parents are expressed equally. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for α and β chains), one gene HLA-DRα (DRA1), and one or more genes HLA-DRβ (DRB1 and DRB3, -4 or -5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB*11:01, HLA-DRB*15:01, and HLA-DRB*07:01.

In some embodiments, the phagocytic cell is administered to a patient or a subject. A cell administered to a human subject must be immunocompatible to the subject, having a matching HLA subtype that is naturally expressed in the subject. Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant. The expression “amino acid” as used herein is intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified by methylation, amidation, acetylation or substitution with other chemical groups which can change the circulating half-life without adversely affecting their biological activity.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. The terms “oligomer” and “oligopeptide” are also intended to mean a peptide as described herein. Furthermore, the term polypeptide extends to fragments, analogues and derivatives of a peptide, wherein said fragment, analogue or derivative retains the same biological functional activity as the peptide from which the fragment, derivative or analogue is derived.

A polypeptide as used herein can be a “protein”, including but not limited to a glycoprotein, a lipoprotein, a cellular protein or a membrane protein. A polypeptide may comprise one or more subunits of a protein. A polypeptide may be encoded by a recombinant nucleic acid. In some embodiments, polypeptide may comprise more than one peptides in a single amino acid chain, which may be separated by a spacer, a linker or peptide cleavage sequence. A polypeptide may be a fused polypeptide. A polypeptide or a protein may comprise one or more domains. A domain is a structural portion of a protein with a defined function, a polypeptide or a protein may comprise one or more modules. A module is domain or a portion of the domain or portion of a protein with a specific function. A module may be a structural module of a protein, designated by its structural embodiments. A moiety is a portion of polypeptide, a protein or a nucleic acid, having a specific structure or perform a specific function. For example, a signaling moiety is a specific unit within the larger structure of the polypeptide or protein or a recombinant nucleic acid, which (or the protein portion encoded by it in case of a nucleic acid) engages in a signal transduction process, for example a phosphorylation. A module, a domain and a moiety, as used herein, can be used interchangeably, unless a specific structural or functional orientation is otherwise defined in the text. A motif is a structural entity in a biomolecule. A signaling motif in a protein or polypeptide, for example, refers to a stretch of amino acids on the protein or polypeptide which contain an amino acid which may be phosphorylated, dephosphorylated or can serve as a binding site of another signaling molecule. Similarly, in case of nucleic acids, for example, TNF mRNA has a conserved motif, UUAUUUAUU, in the 3′UTR to which mRNA destabilizing enzymes such as zinc-finger binding protein 36 family members bind.

The term “pro-antibody” as used herein may refer to an antibody, an scFv, a V_{HH}, single domain antibody, or a protein or polypeptide that comprises an inactive antigen binding domain; wherein the antigen binding capability is designed to be blocked or inactive e.g. by binding a cleavable antigen domain binding polypeptide, until an active step is performed to convert the pro-antibody to its active form. In some embodiments, the active step involves a protease cleavage of the entity that block the antigen binding domain.

As used herein, the term “recombinant nucleic acid molecule” refers to a recombinant DNA molecule or a recombinant RNA molecule. A recombinant nucleic acid molecule is any nucleic acid molecule containing joined nucleic acid molecules from different original sources and not naturally attached together. A recombinant nucleic acid may be synthesized in the laboratory. A recombinant nucleic acid can be prepared by using recombinant DNA technology by using enzymatic modification of DNA, such as enzymatic restriction digestion, ligation, and DNA cloning. A recombinant nucleic acid as used herein can be DNA, or RNA. A recombinant DNA may be transcribed in vitro, to generate a messenger RNA (mRNA), the recombinant mRNA may be isolated, purified and used to transfect a cell. A recombinant nucleic acid may encode a protein or a polypeptide. A recombinant nucleic acid, under suitable conditions, can be incorporated into a living cell, and can be expressed inside the living cell. As used herein, “expression” of a nucleic acid usually refers to transcription and/or translation of the nucleic acid. The product of a nucleic acid expression is usually a protein but can also be an mRNA. Detection of an mRNA encoded by a recombinant nucleic acid in a cell that has incorporated the recombinant nucleic acid, is considered positive proof that the nucleic acid is “expressed” in the cell.

The process of inserting or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the forced introduction of small molecules such as DNA, RNA, or antibodies into eukaryotic cells. Just to make life confusing, ‘transfection’ also refers to the introduction of bacteriophage into bacterial cells. ‘Transduction’ is mostly used to describe the introduction of recombinant viral vector particles into target cells, while ‘infection’ refers to natural infections of humans or animals with wild-type viruses.

As used herein, the term “vector” means any genetic construct, such as a plasmid, phage, transposon, cosmid, chromosome, virus, virion, etc., which is capable transferring nucleic acids between cells. Vectors may be capable of one or more of replication, expression, recombination, insertion or integration, but need not possess each of these capabilities. A plasmid is a species of the genus encompassed by the term “vector.” A vector typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the cell. A vector can be a DNA or RNA vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

The terms “spacer” or “linker” as used in reference to a fusion protein refers to a peptide that joins the proteins comprising a fusion protein. In some embodiments, the constituent amino acids of a spacer can be selected to influence some property of the molecule such as the folding, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. The linker is used to separate two antigenic peptides by a distance sufficient to ensure that, in some embodiments, each antigenic peptide properly folds. Exemplary peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also can be used in the linker sequence.

In some embodiments, the peptide linkers have more than one functional properties, such as the ones described herein. For example, the peptide linker links two or more functional domains, such as binding domains. Additionally, the peptide linker may be a specific signal inducer when the linker contacts an extracellular portion of a cell, such as a receptor or a ligand binding protein.

The term “immunopurification (IP)” (or immunoaffinity purification or immunoprecipitation) is a process well known in the art and is widely used for the isolation of a desired antigen from a sample. In general, the process involves contacting a sample containing a desired antigen with an affinity matrix comprising an antibody to the antigen covalently attached to a solid phase. The antigen in the sample becomes bound to the affinity matrix through an immunochemical bond. The affinity matrix is then washed to remove any unbound species. The antigen is removed from the affinity matrix by altering the chemical composition of a solution in contact with the affinity matrix. The immunopurification can be conducted on a column containing the affinity matrix, in which case the solution is an eluent. Alternatively, the immunopurification can be in a batch process, in which case the affinity matrix is maintained as a suspension in the solution. An important step in the process is the removal of antigen from the matrix. This is commonly achieved by increasing the ionic strength of the solution in contact with the affinity matrix, for example, by the addition of an inorganic salt. An alteration of pH can also be effective to dissociate the immunochemical bond between antigen and the affinity matrix.

As used herein, the terms “determining”, “assessing”, “assaying”, “measuring”, “detecting” and their grammatical equivalents refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

A “fragment” is a portion of a protein or nucleic acid that is substantially identical to a reference protein or nucleic acid. In some embodiments, the portion retains at least 50%, 75%, or 80%, or 90%, 95%, or even 99% of the biological activity of the reference protein or nucleic acid described herein.

The terms “isolated,” “purified”, “biologically pure” and their grammatical equivalents refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

The terms “neoplasia” and “cancer” refers to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

The term “vaccine” is to be understood as meaning a composition for generating immunity for the prophylaxis and/or treatment of diseases (e.g., neoplasia/tumor/infectious agents/autoimmune diseases). Accordingly, vaccines as used herein are medicaments which comprise recombinant nucleic acids, or cells comprising and expressing a recombinant nucleic acid and are intended to be used in humans or animals for generating specific defense and protective substance by vaccination. A “vaccine composition” can include a pharmaceutically acceptable excipient, carrier or diluent. Aspects of the present disclosure relate to use of the technology in preparing a phagocytic cell-based vaccine.

The term “pharmaceutically acceptable” refers to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent. A “pharmaceutically acceptable salt” of pooled disease specific antigens as recited herein can be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluene sulfonic, methane sulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled disease specific antigens provided herein, including those listed by Remington's Pharmaceutical Sciences, 17th ed., Mack Publishing Company, Easton, Pa., p. 1418 (1985).

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 420° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 250° C. in 30 mM NaCl, 3 mM trisodium citrate, and 0.1% SDS. In other exemplary embodiments, wash steps can occur at 420° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Substantially identical” refers to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The terms “treat,” “treated,” “treating,” “treatment,” and the like are meant to refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which refers to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “prevent”, “preventing”, “prevention” and their grammatical equivalents as used herein, means avoiding or delaying the onset of symptoms associated with a disease or condition in a subject that has not developed such symptoms at the time the administering of an agent or compound commences.

The term “therapeutic effect” refers to some extent of relief of one or more of the symptoms of a disorder (e.g., a neoplasia, tumor, or infection by an infectious agent or an autoimmune disease) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required.

As used herein, the term “affinity molecule” refers to a molecule or a ligand that binds with chemical specificity to an affinity acceptor peptide. Chemical specificity is the ability of a protein's binding site to bind specific ligands. The fewer ligands a protein can bind, the greater its specificity. Specificity describes the strength of binding between a given protein and ligand. This relationship can be described by a first scFv specific to a cell surface component on a dissociation constant (KD), which characterizes the balance between bound and unbound states for the protein-ligand system.

Reference in the specification to “some embodiments,” c“an embodiment,” “one embodiment” or “other embodiments” means that a feature, structure, or characteristic described in connection with the embodiments is included in at least some embodiments, but not necessarily all embodiments, of the present disclosure.

The term “myeloid cells” refers to normal or neoplastic cells found in the blood, bone marrow, other hematopoietic or other non-hematopoietic compartments of the body. In particular, the term “myeloid cells” is used herein to mean the cell lineage originating from the bone marrow that includes polymorphonuclear neutrophils, eosinophils, basophils, and mast cells, as well as the monocyte/macrophage lineage and different dendritic cell lineages. Myeloid cells are not capable of differentiating into lymphoid cells (e.g., NK-, B- and T-lymphocytes). The term refers to cells of the myeloid lineages in all stages of their differentiation and therefore includes hematopoietic blast cells, i.e., hematopoietic cells that are committed to the myeloid cell lineage, but that are in early stages of differentiation. When stimulated with appropriate growth factors, hematopoietic blast cells divide to produce a large number of cells that are more differentiated than the blast stage of differentiation. Examples are inter alia myeloblasts. Although monocytes or macrophages are exemplified throughout the specification, the compositions and methods described here are applicable to cells of a myeloid cell lineage, such as a dendritic cell. Minor optimizations and changes are envisioned on a cell to cell basis as is known to one of skill in the art, and is contemplated within the scope of the invention.

Cells that are more differentiated than blasts but not yet fully differentiated are appended with the prefix “pro” and are also intended to fall under the definition of “myeloid cells.” Examples are promyelocytes.

The term “myeloid cells” also includes myeloid progenitor cells, i.e., cell lineages, e.g., in the bone marrow, that are capable of differentiating in cells such as myelomonocytic progenitor cells, proerythroblasts or immature megakaryoblasts. Myeloid progenitor cells are not capable of giving rise to lymphoid cells.

The term “myeloid cells” does not include lympho-hematopoietic stem cells. Lympho-hematopoietic stem cells are defined as those cells that are capable of both self-renewal and differentiation into the two principle precursor components, the myeloid and lymphoid lines. Such stem cells are said to be totipotent. Stem cells that are less general but that can still differentiate into several lines are called pluripotent.

The term “monocyte or macrophage specific engagers” applies to not only monocyte or macrophage cells, but to all myeloid cells, and therefore a monocyte or macrophage specific engager is similar to a “myeloid cell specific engager.”

Phagocytes are the natural sentinels of the immune system and form the first line of defense in the body. They engulf a pathogen, a pathogen infected cell a foreign body or a cancerous cell and remove it from the body. Most potential pathogens are rapidly neutralized by this system before they can cause, for example, a noticeable infection. This can involve receptor-mediated uptake through the clathrin-coated pit system, pinocytosis, particularly macropinocytosis as a consequence of membrane ruffling and phagocytosis. The phagocytes therefore can be activated by a variety of non-self (and self) elements and exhibit a level of plasticity in recognition of their “targets”. Most phagocytes express scavenger receptors on their surface which are pattern recognition molecules and can bind to a wide range of foreign particles as well as dead cell, debris and unwanted particles within the body.

Myeloid cells, such as, monocytes and macrophages are also one of the most abundant cell types in the site of an infection, inflammation or in a tumor. Therefore, monocytes or macrophages can be attractive cell therapy vehicles. Provided herein are mechanisms to modify a monocyte or macrophage or a phagocytic cell to enhance phagocytic killing of a diseased cell, such as a tumor or an infected cell.

Although a monocyte/macrophage is described in detail in the disclosure the composition and methods can be applicable for use in any phagocytic cell type, or applicable towards myeloid cell types including and not limited to neutrophil and dendritic cells with minor optimizations if applicable, as is known to one of skill in the art. Likewise, although cancer is described in detail as the indication for a myeloid cell therapy in the disclosure, the composition and methods can be made applicable to infections and autoimmune conditions, with minor modifications as deemed necessary by a person of skill in the art.

Phagocytosis, defined as the cellular uptake of particulates (>0.5 □m) within a plasma-membrane envelope, is closely relate to and partly overlaps the endocytosis of soluble ligands by fluid-phase macropinocytic and receptor pathways. Variants associated with the uptake of apoptotic cells, also known as efferocytosis, and that of necrotic cells arising from infection and inflammation (necroptosis and pyroptosis). The uptake of exogenous particles (heterophagy) has features in common with autophagy, an endogenous process of sequestration and lysosomal disposal of damaged intracellular organelles. Uptake mechanisms vary depending on the particle size, multiplicity of receptor-ligand interactions, and involvement of the cytoskeleton. Once internalized, the phagosome vacuole can fuse selectively with primary lysosomes, or the product of the endoplasmic reticulum (ER) and Golgi complex, to form a secondary phagolysosome. This pathway is dynamic in that it undergoes fusion and fission with endocytic and secretory vesicles, macrophages, DCs, osteoclasts, and eosinophils. Anti-microbe phagocytosis clears and degrades disease-causing microbes, induces pro-inflammatory signaling through cytokine and chemokine secretion, and recruits immune cells to mount an effective inflammatory response. This type of phagocytosis is often referred to as “inflammatory phagocytosis” (or “immunogenic phagocytosis”). However, in some instances, such as with certain persistent infections, anti-inflammatory responses may follow microbial uptake. Anti-microbe phagocytosis is commonly performed by professional phagocytes of the myeloid lineage, such as immature dendritic cells (DCs) and monocytes or macrophages and by tissue-resident immune cells. Phagocytosis of damaged, self-derived apoptotic cells or cell debris (e.g., efferocytosis), in contrast, is typically a non-inflammatory (also referred to as a “nonimmunogenic”) process. Billions of damaged, dying, and unwanted cells undergo apoptosis each day. Unwanted cells include, for example, excess cells generated during development, senescent cells, infected cells (intracellular bacteria or viruses), transformed or malignant cells, and cells irreversibly damaged by cytotoxic agents.

The bone marrow is the source of circulating neutrophils and monocytes that will replace selected tissue-resident monocytes or macrophages and amplify tissue myeloid populations during inflammation and infection. After phagocytosis, newly recruited monocytes and tissue macrophages secrete their products by generating them from pre-existing phospholipids and arachidonates in the plasma membrane and by releasing radicals generated by activation of a respiratory burst or induction of inducible nitric oxide synthesis; apart from being achieved by synthesis of the low-molecular-weight products (arachidonate metabolites, superoxide anions, and nitric oxide) generated as above, secretion induced by phagocytosis in monocytes or macrophages is mainly achieved by new synthesis of RNA and changes in pH, resulting in progressive acidification. Highly phagocytic macrophages appear to be MARCO+ SignR1+ and are found in the outer marginal zone rapidly clear capsulated bacteria. Similar CD169+ F4/80− macrophages line the subcapsular sinus in lymph nodes and have been implicated in virus infection. It was noted that endothelial macrophages, including Kupffer cells in the liver, clear microbial and antigenic ligands from blood and lymph nodes to provide a sinusoidal immune function comparable to but distinct from mucosal immunity. Not all tissue macrophages are constitutively phagocytic, even though they still express typical macrophage markers. In the marginal zone of the rodent spleen, metallophilic macrophages, which lack F4/80, strongly express CD169, sialic acid-binding immunoglobulin (Ig)-like lectin 1 (SIGLEC1 [sialoadhesin]), but are poorly phagocytic. Non-professional phagocytes include epithelial cells, and fibroblasts. Fibroblasts are “working-class phagocytes” clear apoptotic debris by using integrins other than CD11b-CD18 through adhesion molecules ICAM and vitronectin receptors. Astrocytes have also been reported to engulf, even if not efficiently degrade, apoptotic corpses. Plasma-membrane receptors relevant to phagocytosis can be opsonic, FcRs (activating or inhibitory) for mainly the conserved domain of IgG antibodies, and complement receptors, such as CR3 for iC3b deposited by classical (IgM or IgG) or alternative lectin pathways of complement activation. CR3 can also mediate recognition in the absence of opsonins, perhaps by depositing macrophage-derived complement. Anti-microbe phagocytosis is commonly performed by professional phagocytes of the myeloid lineage, such as immature dendritic cells (DCs) and macrophages and by tissue-resident immune cells.

In cancer, monocytes, attracted by numerous factors including CCL2, ATP, etc., migrate into the tumor microenvironment. However, a majority of these monocytes can then differentiate into tumor associated monocytes or macrophages and or myeloid suppressor cells. In order to generate monocytes or macrophages and myeloid cells that are potent in killing tumor cells, as opposed to being myeloid suppressor cells and tumor associated monocytes or macrophages, the present composition provide means for enhancing phagocytosis of the tumor cells by resident monocytes or macrophages, and also mount a successful and stable immune response.

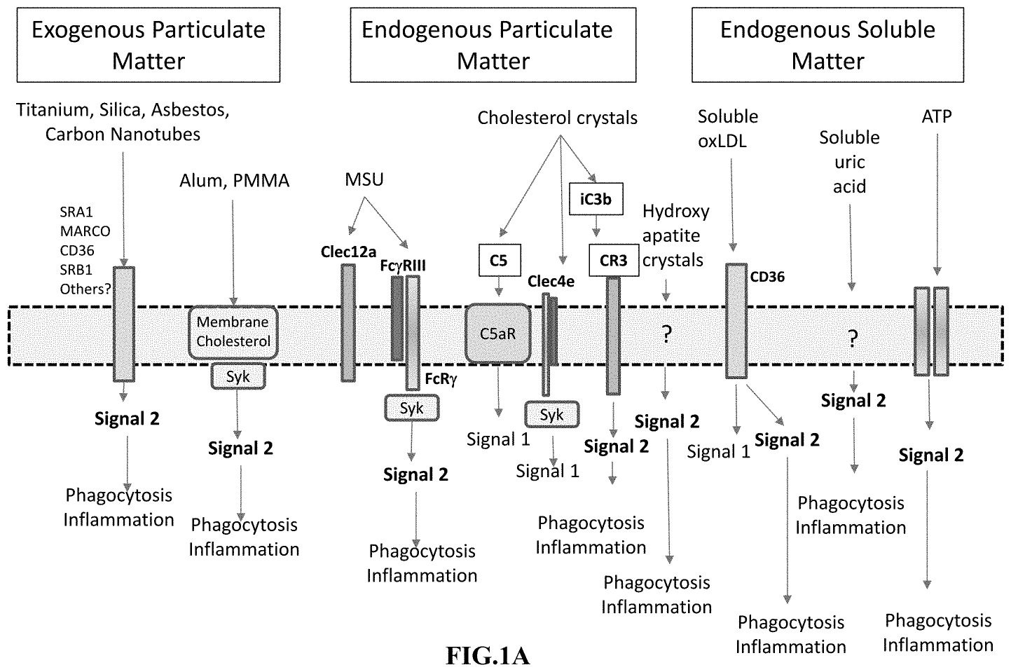

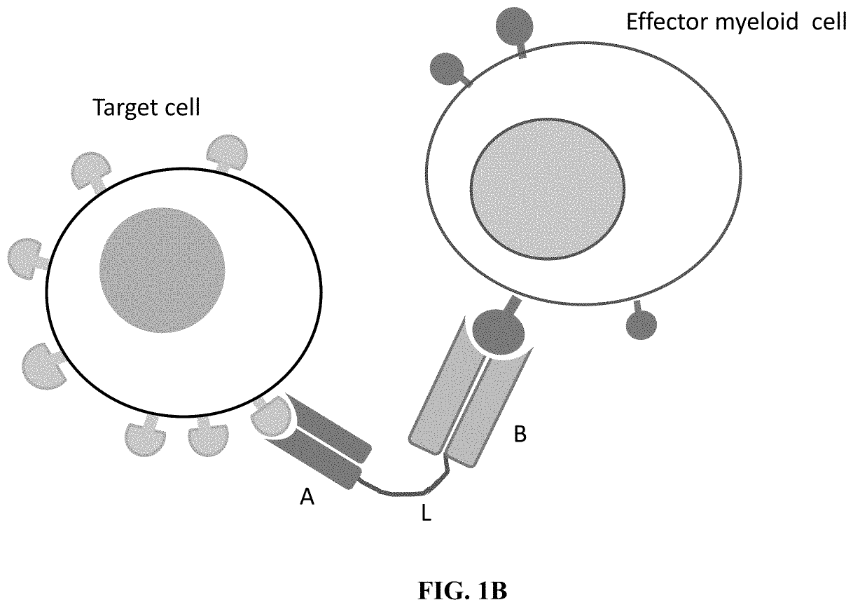

Investigations on monocyte or macrophage function in a tumor environment indicated that at least two signals are required for the activation of monocytes or macrophages. The first signal (signal 1) is mediated via phagocytosis/tethering receptors and the second signal (signal 2) by danger signals such as pathogen-associated molecular patterns (DAMPs), or cytokines that trigger nuclear factor-κB (NF-κB)-mediated upregulation of inflammatory genes (FIG. 1A). Triggering phagocytosis alone may be insufficient to activate monocytes or macrophages and in the context of harnessing macrophages to kill cancer, as it is insufficient to drive an anti-tumor response with a phagocytosis triggering signal alone generated by binding to a cancer cell.

Whereas a cancer cell or a tumor cell is repeatedly referred here as the target cell, the concepts described here are suitable for any type of a target cell, such as an infected cell, or a specific disease cell type that needs to be eliminated by phagocytosis, as long as the binding domain for a cell surface component of a cancer cell is suitably replaced by a binding domain for a cell surface component of the specific for the target cell.

The present disclosure is based on a number of endeavors that address effective ways to trigger a myeloid cell to mount a strong response to a target cell, for example, a cancer cell or tumor cell, such that the myeloid cell destroys the target upon contact, as well as trigger an immune response that activates other immune cells, for example, T lymphocytes, B lymphocytes and NK cells. One aspect of the endeavor is to generate therapeutically effective myeloid cells in the patient, in situ. In another aspect, therapeutic myeloid cells are generated ex vivo, and introduced into a patient in need thereof.

In one aspect, the disclosure provides one or more synthetic or recombinant biomolecules, such as proteins or polypeptides, that are capable of binding to and activating a myeloid cell to trigger phagocytic killing and immune response against a target cell, such as a tumor cell. In some embodiments, the synthetic or recombinant biomolecule can bind (a) on one hand, a cell surface molecule (i.e. and antigen) on a myeloid cell, and on the other hand (b) a cell surface molecule (i.e. and antigen) on a target cell, thereby effectively, at least, bringing the two cells (an effector and a target), in close proximity, such that other cellular receptors and membrane components on either cell can interact and the effector myeloid cell can thereby trigger engulfment of the target cell. A simplified graphical representation is depicted in FIG. 1B. Structurally, such a molecule would have two arms, one specific for each cell surface molecule, for example, a first binding domain (e.g., A), and a second binding domain (e.g., B) connected by a linker (e.g. L) (FIG. 1B). Such synthetic or recombinant biomolecules can be called bispecific engagers, or, bispecific myeloid cell engagers, or BiMEs. In one or more embodiments, the bispecific engagers comprise two antigen binding domains (“binders”). One of the two binders is designed to bind to an antigen on the surface of an effector myeloid cell; the other is designed to bind to an antigen on a target cell. In some embodiments the antigen binding domains are antibodies or fragments thereof. In some embodiments, a binder may be a ligand, binding to a receptor on a cell surface, such as a receptor on a myeloid cell or on a target cell.

In one aspect, the present disclosure provides a therapeutic composition comprising one or more synthetic or recombinant biomolecules, such as proteins or polypeptides, that are capable of binding to and activating a myeloid cell to trigger phagocytic killing and immune response against a target cell, such as a cancer cell, and the synthetic or recombinant biomolecule comprises more than two binders. Accordingly, in some embodiments, provided herein is a first therapeutic agent, wherein the therapeutic agent comprises: a first binding domain (or, a first binder), wherein the first binding domain may be a first antibody or functional fragment thereof that specifically interacts with an antigen or a surface molecule on a target cell, and a second binding domain (or, a second binder), wherein the second binding domain may a second antibody or functional fragment thereof that specifically interacts with a myeloid cell. In one or more embodiments, the first therapeutic agent is coupled to a first component such as a linker or another bioactive peptide that may offer a third binding domain; or an activator molecule, or an additional therapeutic agent. In some embodiments, the composition comprises an additional therapeutic agent.

In one aspect, the disclosure provides one or more synthetic or recombinant biomolecules, such as proteins or polypeptides, that are capable of binding to and activating a myeloid cell to trigger phagocytic killing and immune response against a target cell, such as a cancer cell, and the synthetic or recombinant biomolecule comprises more than two binders. In one embodiment, the recombinant biomolecule comprises three binders each of exhibit specific binding to a surface molecule, and therefore the recombinant biomolecule can exhibit binding to three elements on two or more cells. In one embodiment, the recombinant biomolecule having three binders is capable of binding to more than one antigens on a myeloid cell or on a target cell. A recombinant biomolecule as described here, having three binders is termed a trispecific myeloid cell engager (TriME). In some embodiments, a TriME may bind to, or engage three different cells, for example, a myeloid cell, a target cell such as a cancer cell, and a third cell, or a target. In some embodiments, the BiME or TriME may engage more than one antigens or surface molecules on either a myeloid cell or on a cancer cell that either activates the myeloid cell or inhibits a function of a cancer cell, such as engaging to and inducing tolerance or immunosuppression on a myeloid cell. In some embodiments, a bispecific, trispecific or a multispecific engager may comprise a second trigger, i.e., a second signal that not only induces phagocytosis of the target cell by the myeloid cell, but also initiates an immune response or inflammatory response that activates other immune cells for a prolonged response and generation of immunological memory. In some embodiments, a bispecific, trispecific or a multispecific engager is a chimeric molecule. Accordingly, provided herein is a composition comprising a therapeutic agent, wherein the therapeutic agent comprises: (a) a first binding domain that specifically interacts with an antigen of a target cell, (b) a second binding domain that specifically interacts with a myeloid cell, and (c) a third binding domain that specifically interacts with the myeloid cell. In some embodiments, the composition comprises an additional therapeutic agent. In some embodiments, a binder may also be an activator of a receptor, such as a scavenger receptor, or a TLR receptor. In some embodiments, a binder may be an inhibitor or blocker of a virulent agent on a pathogenic target, or an immune suppressor molecule on a pathogen cell or a tumor cell. In some embodiments, a binding domain or a binder or any part of an engager that may perform a function as described above may be a therapeutic element of the binder. In some embodiments, the engager may comprise one or more therapeutic agents. In some embodiments, the therapeutic composition may comprise one or more engagers, and one or more therapeutic agents, such as a separate recombinant protein, or nucleic acid encoding the same, a pharmaceutical product or a small molecule.

In some aspects as described herein, a second therapeutic agent may be required. A second therapeutic agent may be a second recombinant protein. In some embodiments, the second therapeutic agent can suppress a tumor-mediated immunosuppressor. In some embodiments, the second therapeutic agent is necessary for evading a myeloid cell suppressor function, or a tolerogenic response on myeloid cells. In some embodiments, the second therapeutic is necessary to evade the anti-phagocytic, “don't-eat-me” signals by a tumor cell towards a phagocyte. For example, the second therapeutic may comprise a CD47 antagonist, a CD47 blocker, an antibody, a chimeric CD47 receptor, a sialidase, a cytokine, a proinflammatory gene, a procaspase, or an anti-cancer agent. In some embodiments, the second therapeutic agent can provide the second signal for the phagocytic cell mediated immune response.

Using the methods and compositions described herein, a myeloid cell can be directed to activate the immune response cycle irrespective of the effects in a tumor microenvironment. A phagocytic cell can be directed to phagocytose and kill a target cell, and activate the immune response sequelae that generates successful and sustained adaptive immune response and immunological memory against the target.

In the following section, compositions comprising therapeutic agents are described.

### First Therapeutic Agent

In one aspect, a first therapeutic agent is described, wherein the first therapeutic agent comprises: (i) a first antigen binding domain that specifically interacts with an antigen of a target cell, and (ii) a second antigen binding domain that specifically interacts with an extracellular region of a receptor of a myeloid cell, such as a monocyte or a macrophage cell. The first therapeutic agent is a recombinant chimeric protein, which can bind to at least a target cell, such as a tumor cell, and a monocyte or macrophage cell and attach the two cell types to facilitate phagocytosis of the cancer cell by the monocyte or macrophage. In some embodiments, the first therapeutic agent is a chimeric bi- or trispecific engager. In some embodiments, the first therapeutic agent is coupled to a first component, wherein the first component is an additional therapeutic agent or a third binding domain. An additional therapeutic agent may be a peptide, a protein, a conjugated protein, an antibody, a functional derivative of an antibody such as an scFv, a ligand, a receptor or a functional fragment thereof for a ligand, or a small molecule. In several examples in the preceding sentence, the first therapeutic agent is coupled to a first component, wherein the first component is a binding element that can associate with a cell surface component of the target cell or the monocyte or macrophage cell.

In some embodiments, the first therapeutic agent comprises an additional therapeutic agent. An additional therapeutic agent as described herein can be a small molecule. In some embodiments, the additional therapeutic agent is a peptide binding domain. In some embodiments, the additional therapeutic agent is a cell surface binding domain. In some embodiments, the additional therapeutic agent is a target cell binding domain. In some embodiments, the additional therapeutic agent may be an antibody, a functional derivative of an antibody, such as an scFv. In some embodiments, the additional therapeutic agent is a ligand, a peptide. In some embodiments, the additional therapeutic agent is a protein, a conjugated protein, a receptor or a functional fragment thereof for a ligand. In some embodiments, the additional therapeutic agent is an inhibitor of the myeloid cell, e.g., the monocyte or macrophage cell mediated by the target cell.

In some embodiments, the therapeutic agent is a recombinant protein. The therapeutic agent as described herein is a recombinant protein that not only binds a tumor or a cancer cell and a monocyte or macrophage thereby providing a first signal (signal 1) for triggering phagocytosis of the tumor cell by the monocyte or macrophage, but also provides a second signal (signal 2) to enhance phagocytic killing by the monocyte or macrophage.

In one embodiment the first therapeutic agent is an extracellular protein.

In some embodiments, the first therapeutic agent is a secreted protein.

In some embodiments, the first therapeutic agent is encoded by a recombinant nucleic acid encoding one or more nucleic acid sequences encoding a first antigen binding domain that specifically interacts with an antigen of a target cell, and (ii) a second antigen binding domain that specifically interacts with an extracellular region of a receptor of a monocyte or macrophage cell.

In some embodiments, the first therapeutic agent is encoded by a vector expressing a recombinant nucleic acid encoding one or more polypeptides comprising a first antigen binding domain that specifically interacts with an antigen of a target cell, and (ii) a second antigen binding domain that specifically interacts with an extracellular region of a receptor of a myeloid cell.

In some embodiments, a binder is selected on the basis of its binding specificity to a its target or cognate element. A binding domain may be derived from a protein that is an antibody or a functional fragment thereof, that binds to the target antigen or the cognate molecule. The binding domain is one that has high specificity, high binding affinity or both, towards its target.

In some embodiments, the binding affinity to its target or cognate molecule is 10⁻⁸ M or less, 10⁻⁹ M or less 10⁻¹⁰ M or less or 10⁻¹¹ M or less, 10⁻¹² M or less. In some embodiments, the binding domain may further be modified to increase its binding specificity or binding affinity or both. One of skill in the art can use existing technology to enhance the binding properties of a binder region, and such modifications are contemplated within the scope of this disclosure.

### Bi- and Trispecific Monocyte or Macrophage Engagers

### A. Binding Target Cell and Effector Cell

Provided herein are recombinant bi- and trispecific engagers designed to anchor a target cell with an effector cell, such that the effector cell attack the target cell, and kill the specific target cell. In some embodiments, the effector cell is a myeloid cell. In some embodiments, the myeloid cell is a monocyte or macrophage cell. In some embodiments, the target cell is a cancer cell.

While cancer is one exemplary embodiment described in exclusive detail in the instant disclosure, the methods and technologies described herein are contemplated to be useful in targeting an infected or otherwise diseased cell inside the body.

In some embodiments, the present disclosure provides compositions and methods for cancer immunotherapy. The methods provided herein help design tools that can induce resident human monocytes or macrophages to become efficient killer cells that target cancer cells and eliminate them by efficient phagocytosis. In some embodiments, the monocytes or macrophages provide sustained immunological response against the cancer cell. Various embodiments are described herein.

Provided herein are specific constructs and designs are disclosed for such chimeric proteins, termed chimeric “engagers”.

In some embodiments, the chimeric engagers comprise two or more fused antibodies, each having a specific binding region on the target cell, such as cancer cell or on the monocyte or macrophage. In certain embodiments, the two or more fused antibodies or the immunofusion comprises a target binding domain operably linked by a hinge-CH2-CH3 domain or a hinge-CH3 domain of an immunoglobulin constant region to an effector binding domain that specifically binds a cell surface component of the monocyte or macrophage.

In one aspect the chimeric protein is a bispecific monocyte or macrophage engager.

In some embodiments, a bispecific engager comprises a first therapeutic agent, wherein the first therapeutic agent comprises: (i) a first antigen binding domain that specifically interacts with an antigen of a target cell, and (ii) a second antigen binding domain that specifically interacts with an extracellular region of a receptor of a monocyte or macrophage cell. In one embodiment, the therapeutic agent is a bispecific engager. In one embodiment, the bispecific monocyte or macrophage engager comprises two antibody single chain variable regions (scFv) only (no Fc amino acid segments were included) with a flexible linker, one scFv binds a cell surface component of a target cell and the other binds a receptor on monocyte or macrophage cell surface. In full unmodified forms of IgG, the variable light chain domain (V_{L}) and the variable heavy chain domain (VH) are separate polypeptide chains, i.e., are located in the light chain and heavy chain, respectively. Interaction of the antibody light chain and an antibody heavy chain, in particular the interaction of the V_{L} and V_{H} domains, one of the epitope binding site of the antibody is formed. In contrast, in the scFv construct, but V_{L} and V_{H} domains of antibodies are included in a single polypeptide chain. The two domains are separated by flexible linkers long enough to allow self-assembly of the V_{L} and V_{H} domains into functional epitope binding site.

In some embodiments, a bispecific monocyte or macrophage engager comprises: (a) a single chain variable fragment (scFv) that binds to a cell surface component of a target cell, e.g., a cancer antigen, (b) a single chain variable fragment (scFv) that binds to a cell surface component of an effector cell, e.g. the monocyte or macrophage, (c) a short linker operably linking (a) and (b). In some embodiments, the scFvs are fused at their C-termini. Each scFv comprises a light chain variable domain, and a heavy chain variable domain, operably linked by a peptide linker. In certain embodiments, the scFvs are humanized. Humanized scFvs comprise “complementarity determining regions” (CDR) that are present on a framework of an immunoglobulin of a different species as compared to that of the parent immunoglobulin from which the CDR was derived. For example, a murine CDR may be grafted into the framework region of a human antibody to prepare the “humanized antibody.” The design of an exemplary bispecific engager comprising two scFvs can be represented by the simplified formula:

NH2-[Target cell binding scFv]-COOH-[Linker]-COOH-[Effector cell binding scFv]-NH2 [I]

In some embodiments, the bispecific engager is a diabody. The bispecific diabody is constructed with a V_{L} and a V_{H} domain on a single polypeptide chain have binding specificities to different (non-identical) epitopes. Additionally, the linker connecting V_{L} and V_{H} is shorter than 12 amino acid in length that is insufficient for reassembly into a functional epitope. Generally, one polypeptide chain construct comprises V_{L} having binding specificity to a first antigen and V_{H} having binding specificity to a second antigen, and another polypeptide chain construct comprises V_{L} having binding specificity to the second antigen and V_{H} having binding specificity to the first antigen; the two polypeptide chains are allowed to self-assemble into a bi-specific diabody. In some embodiments, a cysteine residue may be introduced at the C terminus of the construct that can allow disulfide bond formation between two chains without interfering with the binding properties of the

In some embodiments, the bispecific engager is a tandem-di-scFv.

In some embodiments, recombinant nucleic acid constructs can be prepared encoding the bispecific scFv engager. The recombinant nucleic acid constructs for expressing a bispecific scFv engager comprises one or more polypeptides encoding (a) a nucleic acid sequence encoding a variable domain of the target cell binding scFv light chain, a linker, a variable domain of the target cell binding scFv heavy chain; (b) a nucleic acid sequence encoding a linker; (c) a nucleic acid sequence encoding a variable domain of the effector (monocyte or macrophage) cell binding scFv light chain, a linker, a variable domain of the effector (monocyte or macrophage) cell binding scFv heavy chain. In some embodiments, the nucleic acid constructs for expressing a bispecific scFv engager comprises an N-terminal signal peptide sequence for secretion of the bispecific scFv engager.

In some embodiments, a bispecific engager comprises two single domain antibodies (V_{HH}) operably linked with a flexible linker, one V_{HH} binds a cell surface component of a target cell, and the other V_{HH} binds a receptor on a monocyte or macrophage cell surface. In some embodiments, a chimeric bispecific monocyte or macrophage engager comprises: (a) a V_{HH} domain that binds to a cell surface component of a target cell, e.g., a cancer antigen, (b) a V_{HH} domain that binds to a cell surface component of an effector cell, e.g. the monocyte or macrophage, (c) a short linker operably linking (a) and (b). In some embodiments the engager comprising two single domain antibodies is a nanobody. The design of an exemplary bispecific engager comprising two V_{HH} domains can be represented by the simplified formula:

NH2-[Target cell binding single domain]-COOH-[Linker]-COOH-[Effector cell binding single domain]-NH2 [II]

In some embodiments, the short linker operably linking (a) and (b) may further have additional functions. In some embodiments, the peptides can bind to a specific cell surface receptor, such as, for example, a TLR receptor, and can activate a receptor mediated cell signaling pathway in the monocyte or macrophage cell. In some embodiments, the linker is designed such as to be able to bind and activate at least an inflammatory pathway in the monocyte or macrophage cell, or potentiate monocyte or macrophage mediated phagocytosis and killing of a target cell. In some embodiments, the linker peptide may have a function of blocking or inhibiting a target cell mediated downregulation of a monocyte or macrophage cell function.

In some embodiments, nucleic acid constructs for a bispecific V_{HH} engager can be generated, which comprises: (a) a nucleic acid sequence encoding a (a) a V_{HH} domain that binds to a cell surface component of a target cell, e.g., a cancer antigen, (b) a V_{HH} domain that binds to a cell surface component of an effector cell, e.g. the monocyte or macrophage, (c) a short linker operably linking (a) and (b). In some embodiments, the nucleic acid constructs for expressing a bispecific scFv engager comprises an N-terminal signal peptide sequence for secretion of the bispecific scFv engager.

As is known to one of skill in the art, the nucleic acid sequences encoding the polypeptides comprising the V_{HH} or scFv binding domains can be inserted in a suitable expression vector under one or more promoters, e.g. CMV at the 5′ end, and a polyadenylation signal at the 3′-end of the sequences encoding the polypeptides.

In some embodiments, the constructs may comprise internal ribosomal entry site (IRES), e.g., a nucleic acid sequences encoding one or more polypeptides may be preceded by an IRES.

In some embodiments, the nucleic acid sequences encoding one of the polypeptides may be placed under a separate promoter control than the remaining of the expressed sequences.

In some embodiments, a bispecific engager may further comprise an antibody or a fragment thereof that binds to a cell surface component of a target cell, and an antibody or a fragment thereof that binds to a cell surface component of an effector cell.

Provided herein are further variations of an engager, a trispecific engager. A trispecific engager comprises a first therapeutic agent, wherein the first therapeutic agent comprises: a first antigen binding domain that specifically interacts with an antigen of a target cell; a second antigen binding domain that specifically interacts with an extracellular region of a first receptor of a monocyte or macrophage cell; and a third antigen binding domain that specifically interacts with an extracellular region of a second receptor of the monocyte or macrophage cell.

In some embodiments, the trispecific engager is a fused construct of three scFvs, comprising a first scFv specific to a cell surface component on a target cancer cell, a second scFv specific to a cell surface component on the monocyte or macrophage, for example, the chimeric phagocytic receptor, and a third scFv specific to another cell surface component on the monocyte or macrophage. In some embodiments, the trispecific engager is designed such that the cell surface component on the monocyte or macrophage to which the third scFv can bind, provides an additional activation signal for the monocyte or macrophage to trigger phagocytosis and killing of the target cell. In some embodiments the third scFv binds to another phagocytic receptor on the monocyte or macrophage. In some embodiments the third scFv binds to a danger associated monocyte or macrophage signaling pathway (DAMP). In some embodiments, the third scFv binds to a TLR receptor. In some embodiments, the third scFv binds to a cytokine receptor which activates the receptor and triggers monocyte or macrophage intracellular signaling. In some embodiments, the third scFv binds to a monocyte or macrophage receptor known to generate a phagocytosis inhibitory signal and that binding of the third scFv to the receptor blocks the receptor, enabling enhanced phagocytosis. In some embodiments, the third scFv binds to a receptor that engages with one or more transmembrane domains and enhances phagocytic signaling. Various designs of trispecific engagers have been contemplated herein, of which an exemplary trispecific engager comprising two scFvs can be represented by the simplified formulae:

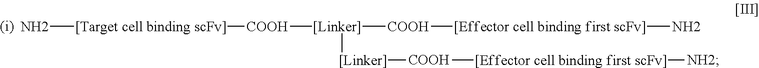

OR

(ii)NH2-[Target cell binding scFv]-COOH-[Linker]-COOH-[Effector cell binding first scFv]-NH2-[Linker]-COOH-[Effector cell binding second scFv]-NH2. [IV]

In some embodiments, each of the three binding domains of the trispecific engager comprises the antigen binding domain of an antibody, a functional fragment of an antibody, a variable domain thereof, a V_{H} domain, a V_{L} domain, a VNAR domain, a V_{HH} domain, a single chain variable fragment (scFv), an Fab, a single-domain antibody (sdAb), a nanobody, a bispecific antibody, a diabody, or a functional fragment or a combination thereof.

In some embodiments, the binding domains of the trispecific engager are operably linked by one or more peptide linkers. In some embodiments, the one or more peptide linkers may be functional peptides that can bind to a specific cell surface receptor, such as, for example, a TLR receptor, and can activate a receptor mediated cell signaling pathway in the monocyte or macrophage cell. In some embodiments, the linker is designed such as to be able to bind and activate at least an inflammatory pathway in the monocyte or macrophage cell, or potentiate monocyte or macrophage mediated phagocytosis and killing of a target cell. In some embodiments, the linker peptide may have a function of blocking or inhibiting a target cell mediated downregulation of a monocyte or macrophage cell function.

In some embodiments, a nucleic acid constructs encoding a trispecific engager comprises one or more nucleic acid encoding (a) a polypeptide comprising an scFv domain that binds to a cell surface component of a target cell, e.g., a cancer antigen, (b) a polypeptide comprising an scFv domain that binds to a first cell surface component of an effector cell, e.g. the monocyte or macrophage, (c) a polypeptide comprising an scFv domain that binds to a second cell surface component of the monocyte or macrophage, for example, the chimeric construct constituting the second therapeutic agent; or a native monocyte or macrophage cell surface receptor, wherein each of the polypeptides are operably linked to one another. In some embodiments, a nucleic acid constructs encoding a trispecific engager comprises one or more nucleic acid encoding (a) a polypeptide comprising a V_{HH} domain that binds to a cell surface component of a target cell, e.g., a cancer antigen, (b) a polypeptide comprising a V_{HH} domain that binds to a first cell surface component of an effector cell, e.g. the monocyte or macrophage, (c) a polypeptide comprising a V_{HH} domain that binds to a second cell surface component of the monocyte or macrophage. In some embodiments, the nucleic acid constructs for expressing a bispecific scFv engager comprises an N-terminal signal peptide sequence for secretion of the bispecific scFv engager. As contemplated herein, a skilled artisan can exchange the scFv or V_{HH} binding sequences with a nucleic acid sequence of a short peptide encoding any suitable target region binding element. In some embodiments, the polypeptide constructs are encoded in a monocistronic construct. In some embodiments, the polypeptide constructs are encoded in a polycistronic construct. In some embodiments, one or more nucleic acid sequences encoding short linker polypeptides are inserted in between sequences encoding two polypeptides. In some embodiments, the expression of the nucleic acid sequence encoding each polypeptide is driven by a separate promoter. In some embodiments, the nucleic acid sequence encoding each polypeptide is driven by a single promoter. In some embodiments one or more IRES sequences are introduced into the construct.

In some embodiments, one or more polypeptides may be expressed separately within a cell, and which may assemble post-translationally.

In some embodiments, polypeptides may be designed to assemble on special peptide scaffolds upon secretion outside the cell.

In some embodiments, the bi- or trispecific engagers bind to an antigen on a cancer cell, selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor vIII (EGFRvIII), Mesothelin, Human Epidermal Growth Factor Receptor 2 (HER2), Mesothelin, EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFR-beta, SSEA-4, EGFR, NCAM, prostase, PAP, ELF2M, GM3, TEM7R, CLDN6, TSHR, GPRC5D, ALK, IGLL1 and combinations thereof. In some embodiments, for example, the cancer antigen for a target cancer cell can be one or more of the mutated/cancer antigens: MUC16, CCAT2, CTAG1A, CTAG1B, MAGE A1, MAGEA2, MAGEA3, MAGE A4, MAGEA6, PRAME, PCA3, MAGE C1, MAGEC2, MAGED2, AFP, MAGEA8, MAGE9, MAGEA11, MAGEA12, IL13RA2, PLAC1, SDCCAG8, LSP1, CT45A1, CT45A2, CT45A3, CT45A5, CT45A6, CT45A8, CT45A10, CT47A1, CT47A2, CT47A3, CT47A4, CT47A5, CT47A6, CT47A8, CT47A9, CT47A10, CT47A11, CT47A12, CT47B1, SAGE1, and CT55.

In some embodiments, the antigen on a cancer cell is selected from the group consisting of CD2, CD3, CD4, CD5, CD7, CCR4, CD8, CD30, CD45, CD56.

In some embodiments, the antigen is an ovarian cancer antigen or a T lymphoma antigen.

In some embodiments, for example, the cancer antigen for a target cancer cell can be one or more of the mutated/cancer antigens: IDH1, ATRX, PRL3, or ETBR, where the cancer is a glioblastoma.

In some embodiments, for example, the cancer antigen for a target cancer cell can be one or more of the mutated/cancer antigens: CA125, beta-hCG, urinary gonadotropin fragment, AFP, CEA, SCC, inhibin or extradiol, where the cancer is ovarian cancer.

In some embodiments the cancer antigen for a target cancer cell may be CD5.

In some embodiments the cancer antigen for a target cancer cell may be HER2.

In some embodiments the cancer antigen for a target cancer cell may be EGFR Variant III.

In some embodiments the cancer antigen for a target cancer cell may be CD19.

In some embodiments, the antigen is an integrin receptor.

In some embodiments, the antigen is an integrin receptor selected from the group consisting of α1, α2, αIIb, α3, α4, α5, α6, α7, α8, α9, α10, α11, αD, αE, αL, αM, αV, αX, β1, β2, β3, β4, β5, β6, β7, and β8. In some embodiments, the bi- or trispecific engager binds to an extracellular domain of a monocyte or macrophage receptor from a member of the integrin β₂ subfamily α_{M}β₂ (CD11b/CD18, Mac-1, CR3, Mo-1), α_{L}β₂ (CD11a/CD18, LFA-1), α_{X}β₂ (CD11c/CD18), and α_{D}β₂ (CD11d/CD18).

Provided herein are exemplary target cell binders (e.g., engagers) that can specifically bind to a cell surface molecule (such as a cell surface antigen) on a cancer cell. In some embodiments, the binder is an antibody specific to the antigen, or a fragment thereof. In some embodiments, the binder comprises a scFv, or a fragment thereof, that specifically binds to an antigen on a tumor cell. In some embodiments, the antigen on a tumor cell is CD5. The binder comprises a heavy chain (HC) sequence and a light chain (LC) sequence. An scFv specific for CD5 (anti-CD5 scFv) comprises an amino acid sequence corresponding to a variable heavy chain (VH) domain and an amino acid sequence corresponding to a (V_{L}). In some embodiments, a first binding domain, which is a CD5 binder can be an scFv having comprising a sequence of SEQ ID NO: 27, and a sequence of SEQ ID NO: 28, joined by a linker peptide. Provided herein in Table 1A are exemplary anti-CD5 HC and LC variable domains.

TABLE 1A
Exemplary CD5 binder domains

| Domain | Sequence |
|---|---|
| Anti-CD5 | EIQLVQSGGGLVKPGGSVRISCAASGYTFTNYGMNWVRQAPGKG |
| heavy chain | LEWMGWINTHTGEPTYADSFKGRFTFSLDDSKNTAYLQINSLRAE |
| variable | DTAVYFCTRRGYDWYFDVWGQGTTVTV |
| domain | (SEQ ID NO: 27). |
| Anti-CD5 light | DIQMTQSPSSLSASVGDRVTITCRASQDINSYLSWFQQKPGKAPKT |
| chain variable | LIYRANRLESGVPSRFSGSGSGTDYTLTISSLQYEDFGIYYCQQYD |
| domain | ESPWTFGGGTKLEIK |
| | (SEQ ID NO: 28). |

In one embodiment, the target cell binder is a single domain antibody that binds CD5. In some embodiments the target cell binder is a CD5-binding V_{HH}. In some embodiments, the target cell binder or a first binding domain can be a CD5 binding V_{HH} comprising a sequence of SEQ ID NO: 27.

In some embodiments, an exemplary target cell binder (e.g., an engager) is a HER2 engager, that can specifically bind to cell surface antigen HER2 on a HER2 positive cancer cell. In some embodiments, the binder is an antibody specific to the antigen, or a fragment thereof. In some embodiments, the binder comprises a scFv, or a fragment thereof, that specifically binds to HER2. The binder comprises a heavy chain (HC) sequence and a light chain (LC) sequence. An scFv specific for HER2 (anti-HER2 scFv) comprises an amino acid sequence corresponding to a variable heavy chain (VH) domain and an amino acid sequence corresponding to a (V_{L}). In some embodiments, a first binding domain may be an scFv having a HER2 binder comprising a sequence of SEQ ID NO: 29, and a sequence of SEQ ID NO: 30, joined by a linker peptide. In some embodiments, the target cell binder or a first binding domain can be a HER2 binding V_{HH} comprising a sequence of SEQ ID NO: 29.

Provided herein in Table 1B are exemplary anti-HER2 HC and LC variable domains.

TABLE 1B
Exemplary HER2 binder domains

| Domain | Sequence |
|---|---|
| Anti-HER2 | DIQMTQSPSSLSASVGDRVTITCRASQDVNTAVAWYQQKPGKAP |
| heavy chain | KLLIYSASFLYSGVPSRFSGSRSGTDFTLTISSLQPEDFATYYCQQH |
| variable | YTTPPTFGQGTKVEIKRTGSTSGSGKPGSGEGSEVQLVE |
| domain | (SEQ ID NO: 29). |
| Anti-HER2 | LVQPGGSLRLSCAASGFNIKDTYIHWVRQAPGKGLEWVARIYPTN |
| light chain | GYTRYADSVKGRFTISADTSKNTAYLQMNSLRAEDTAVYYCSRW |
| variable | GGDGFYAMDVWGQGTLVTV |
| domain | (SEQ ID NO: 30). |

In some embodiments, the tumor associated macrophages may be characterized largely as having an M2 phenotype. Human M2 macrophages can be identified as nearing the cell surface markers CD14+CD163+CD206+CD80− phenotype. Hence, a bi- or trispecific engager that specifically binds to the myeloid cell, e.g., a monocyte or macrophage associated with a tumor can comprise one or more binding domains that can bind to one or more of: CD14, CD163, and CD206 cell surface molecules.

Typically, the M2-like tumor associated macrophage (TAM) population lacks expression of reactive nitrogen intermediates, less efficiently presents antigen, displays little tumoricidal activity, and produces angiogenic factors, metalloproteases, and cathepsins. Matrix metalloproteinases, e.g., MMP2 is readily expressed in TAMs. Classical activation of macrophages up-regulate MMP-1, -3, -7, -10, -12, -14 and -25 and decrease TIMP-3 (tissue inhibitors of metalloproteinase-3) levels. Bacterial lipopolysaccharide, IL-1 and TNFα are found to be more effective than IFN-gamma except for the effects on MMP-25, and TIMP-3. By contrast, alternative activation decrease MMP-2, -8 and -19 but increase MMP-11, -12, -25 and TIMP-3 steady-state mRNA levels. Up-regulation of MMPs during classical activation depends on mitogen activated protein kinases, phosphoinositide-3-kinase and inhibitor of KB kinase-2. Therefore, depending on the target monocyte or macrophage population, an engager may be designed such that a metalloproteinase can be a binding moiety for the monocyte or macrophage engager. MMP2 being one of the readily expressed TAM markers, a tumor specific myeloid cell engager comprises a MMP2 binding domain.

Hypoxia, or cytokines produced secondary to hypoxia, attract macrophages which subsequently up-regulate hypoxia inducible factor 2-alpha (HIF-2α). Accordingly, a binding domain on a bi- or trispecific engager that specifically binds to a tumor associated macrophage can bind to HIF-2α which is upregulated in these cells.

Monocyte/Macrophage cell-surface markers include LPS co-receptor (CD14), HLA-DR (MHC class II), CD312, CD115, the Fcγ-receptor FcγRIII (CD16). Subset-specific markers include CD163 and CD204, both scavenger receptors expressed by M2 macrophages, CD301, a galactose-type C-type lectin expressed by M2 macrophages.

In some embodiments, the bi- or trispecific engager binds to an extracellular domain of a phagocytic receptor, selected from the extracellular domains of any one of the proteins in Table 2A.

TABLE 2A
Exemplary receptors on phagocytes

| Gene names, aliases | NCBI Acc # |
|---|---|
| MSR1, SR-AI, , CD204, SCARA1, SR-A1 | NM_138715 |
| Alternatively spliced form of SR-AI SR-AII SR-A1.1 | NM_002445 |
| MARCO, SCARA2, SR-A6 | NM_006770 |
| SCARA3, MSRL1, SR-A3 | NM_016240 |
| COLEC12, SCARA4, SRCLI, SRCLII, CL-P1, SR-A4 | NM_130386 |
| SCARA5, TESR, NET33 SR-A5 | NM_173833 |
| CD36 SCARB3, FAT, GPIV, PAS4 SR-B2 | NM_001001548 |
| SCARB1 SR-BI, CD36L1 SR-B1 | NM_005505 |
| CD68 gp110, SCARD1, LAMP4 SR-D1 | NM_001251 |
| OLR1 LOX-1, SCARE1, CLEC8A SR-E1 | NM_002543 |
| Alternatively spliced form of SRE-1 LOXIN SR-E1.1 | NM_001172632 |
| CLEC7A, Dectin-1, SCARE2, CD369, SR-E2 | NM_197947 |
| CD206/MRC1, Mannose receptor 1 SR-E3 | NM_002438 |
| ASGPR ASGR1, CLEC4H1, HL-1 SR-E4 | NM_001197216 |
| SCARF 1, SREC-I, SR-F1 | NM_003693 |
| MEGF10, EMARDD, SR-F2 | NM_032446 |
| CXCL16, SR-PSOX SR-G1 | NM_001100812 |
| STAB1, FEEL-1, SR-H1 | NM_015136 |
| STAB2, FEEL-2, SR-H2 | NM_017564 |
| CD163 M130, CD163A, SR-I1 | NM_004244 |
| CD163L1 CD163B, M160 SR-I2 | NM_001297650 |
| SCART1 CD163c-a SR-I3 | NR_002934.3 |
| RAGE (membrane form) AGER SR-J1 | NM_001136 |
| RAGE (soluble form) AGER SR-J1.1 | AB061668 |
| CD44 Pgp-1 SR-K1 | NM_000610 |
| LRP1 A2MR, APOER, CD91 SR-L1 | NM_002332 |
| LRP2 Megalin, gp330 SR-L2 | NM_004525 |
| SRCRB4D | NM_080744 |
| SSC5D | NM_001144950 |
| CD14 | NM_000591 |
| Ly75/CD205 | NM_002349 |
| CD207/Langerin | NM_015717 |
| CD209/DC-SIGN CLEC4L | NM_021155 |

In some embodiments, the bi- or trispecific engager binds to an extracellular domain of a PFP, selected from the extracellular domains of any one of the proteins in Table 2B.

Table 2B provides exemplary surface markers and phenotypic characteristics of monocytes, macrophages and DCs.

TABLE 2B

| | Molecularly defined | Other characteristics |
|---|---|---|
| Monocytes | CD14+ + CD16− | CD16+ monocytes |
| | CD14+ + CD16+ | (undefined as to |
| | DC-like phenotype - | whether they |
| | High | CD14+ + CD16+ or |
| | DR, CD80+ | CD16+ CD14dim) |
| | Macrophage-like | possess superior |
| | phenotype - CD163+, | phagocytosis compared |
| | CD68+ | to blood monocytes and |
| | CD16+ CD14dim | can efficiently activate |
| | CD14 “DC”-Postulated | CD4+ T cells |
| | to be monocyte derived | |
| Macrophages in | Pan CD68 | Liver Macrophages |
| the liver | | appear to |
| | | be predominantly |
| | | tolerogenic in |
| | | nature, with a |
| | | regulatory and |
| | | scavenging role |
| Dendritic cells | BDCA1 (CD1c+) DC | Tolerogenic in nature; |
| | BDCA2 (CD303+) DC | Lower expression of |
| | BDCA3 (CD141hi) DC | costimulation markers |
| | | compared to spleen; |
| | | Produce IL-10 on LPS |
| | | stimulation; Stimulate |
| | | T-cells that are |
| | | IL-10 producing and |
| | | hypo-responsive |
| | | on re-stimulation; |
| | | Produce higher |
| | | numbers of FoxP3+ |
| | | Treg cells on naïve |
| | | T cell stimulation; |
| | | Weak MLR response |
| | | compared to blood. |

In some embodiments, the bi- or trispecific engager binds to an extracellular domain of a myeloid cell receptor, e.g., a monocyte receptor, a macrophage receptor, for examples, a receptor selected from the extracellular domain comprises an Ig binding domain.

In some embodiments, the bi- or trispecific engager binds to an extracellular domain of a macrophage receptor e.g., an IgA, IgD, IgE, IgG, IgM, FcγRI, FcγRIIA, FcγRIIB, FcγRIIC, FcγRIIIA (CD16), FcγRIIIB, FcRn, FcRL5 binding domain. A CD16 receptor referred to herein can be a CD16A receptor or a CD16B receptor.

In some embodiments, the bi- or trispecific engager binds to an extracellular domain of a an FcR extracellular domain.

In some embodiments, the bi- or trispecific engager binds to an extracellular domain of a macrophage receptor selected from the extracellular domains of an FcR-alpha, an FcR-beta, an FcR-Epsilon or an FcR-gamma.

In some embodiments, the bi- or trispecific engager binds to an extracellular domain of an FcαR (FCAR).

In some embodiments, the bi- or trispecific engager binds to an extracellular domain of an FcR-beta.

In some embodiments, the bi- or trispecific engager binds to an extracellular domain of an FcεR (FCER1A).

In some embodiments, bi- or trispecific engager binds to the extracellular domain comprises an FcγR (FDGR1A, FCGR2A, FCGR2B, FCGR2C, FCGR3A, FCGR3B) receptor.

In some embodiments, the bi- or trispecific engager binds to an extracellular domain of a monocyte or macrophage phagocytic receptor selected from selected from lectin, dectin 1, mannose receptor (CD206), scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), and CD169 receptor.

In some embodiments, the bi- or trispecific engager binds to the extracellular domain of a TREM protein. In some embodiments, the extracellular domain of a TREM protein is a TREM 1 protein extracellular domain. In some embodiments, the extracellular domain of a TREM protein is a TREM 2 protein extracellular domain. In some embodiments, the extracellular domain of a TREM protein is a TREM 3 protein extracellular domain.

In some embodiments, the bi- or trispecific engager binds to an extracellular domain of a monocyte or macrophage receptor selected from a group consisting of lectin, dectin 1, mannose receptor (CD206), scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, CCR2, CX3CR1, CSF1R, Tie2, HuCRIg(L), and CD169 receptor.

It may be understood that in some embodiments, a binder or any part of an engager can be a molecule other than an antibody or a fragment thereof. For example, a binder that binds to a surface molecule of a cell, such as a target cell or an effector myeloid cell, and may be a ligand for a receptor, where the cell surface molecule is a receptor specific for the ligand. In some embodiments a ligand may be a chimeric protein or a fusion protein, or a naturally occurring ligand of the receptor. In some embodiments one binder in an engager recombinant protein may be a ligand, and another may be an antibody or a fragment thereof.

In some embodiments, an engager molecule may comprise one or more linkers or spacers. Linkers or spacers may be made up of 2-50 amino acids. In some embodiments, the linker is a 3-30 amino acid spacer. In some embodiments, the linker is a 4-20 or 5-10 amino acid long peptide. In some embodiments, the spacer may be made of nonreactive amino acid moieties, for example, a series of glycine or serine or alanine residues. An exemplary linker may comprise an amino acid sequence GSGS, or SGGG, or SGGGGSG. An exemplary linker may comprise an amino acid sequence SSGGGGSGGGGSGGGGS. A linker may link the V_{H} and V_{L} domains of an scFv. A linker may link the binder domains of a bi- or trispecific engager. The linkers generally serve as structural elements that connect the effective binder sites. In some embodiments, the linker may be flexible. In some embodiments, the linker may be rigid. The length of the linker is adjusted as per the need of the design and the length that is optimal or necessary to space the binders at the opposite ends. In some embodiments, the linker may comprise a peptide that has a unique function, other than linking two domains. Exemplary peptides discussed below may be part of the design of a bi- or trispecific or multispecific engager, and may or may not be a part of a linker.

In some embodiments, the bi- or trispecific engager comprises a peptide that specifically targets the tumor associated macrophages, such as, for instance, the M2 macrophages. In some embodiments, the M2-specific peptide is M2-pep, having an amino acid sequence, YEQDPWGVKWWY. (SEQ ID NO: 116).

In some embodiments, an M2-specific peptide may comprise a sequence HLSWLPDVVYAW, (hereafter, HLS pep) (SEQ ID NO: 117).

A peptide such as the M2-pep or the HLS pep described above can form a part of the bi-specific engager or a trispecific engager, such as a linker between two binding domains or a part of a linker. In some embodiments, the first and the second binding domains of an engager are coupled to an M2-pep or an HLS pep, whereas the M2-pep or the HLS pep further target the engager to the tumor associated macrophages, and help tether the engager to the tumor associated macrophages.

In some embodiments, an exemplary engager may comprise a CD5 binding domain and a CD16 binding domain, connected by a linker. In some embodiments an exemplary engager comprises TLR activation peptide, such as a TLR4 peptide.

B. Engagers with Masked Antigen Binding Domains

Provided herein are compositions for a therapeutic agent that comprising bispecific or trispecific engagers comprising one or more pro-antibodies. In some embodiments, a pro-antibody is an inactive form of an antibody, or fragments or variants thereof, whose antigen binding domain is blocked or “masked” from interacting with the antigen. In some embodiments, the pro-antibody comprises a substrate peptide or a conjugate that remains associated with the antigen binding domain by a protease cleavable linker peptide and “mask” the antigen binding domain from binding to its cognate antigen. Under suitable condition, the substrate peptide is cleaved to release the mask, and promote antigen binding at the antigen binding domain.

In some embodiments, the bispecific or trispecific antibody comprises one or more scfv that is a pro-antibody, that is, the antigen binding domain of the scFv is masked with a cleavable blocker. In some embodiments the blocker comprises a substrate peptide and a protease cleavable linker. In some embodiments, the bispecific or trispecific antibody comprises a V_{HH} pro-antibody, wherein, the V_{L} domain or the V_{H} domain or both of the V_{HH} antibody is masked with a cleavable blocker. In some embodiments, the bispecific or trispecific antibody comprises a nanobody where one or more of antigen binding domains are masked by association with a substrate peptide or conjugate linked to the antibody by a cleavable linker.

In some embodiments, the cleavable linker is designed such that it is cleaved when the therapeutic agent reaches the target site of its action. For example, the pro-antibody for a cancer therapeutic agent can be designed to contain a protease cleavable linker where the protease that cleaves the protease cleavable linker is abundant in the tumor microenvironment and relatively absent or negligible in the non-tumor tissue, and the therapeutic agent is activated by the protease when the therapeutic agent reaches the tumor microenvironment when administered systemically. In some embodiments, therapeutic agents are developed comprising protease-activated pro-antibodies to direct antibody action solely to disease sites.

In some embodiments, the cleavable linker is a matrix metalloprotease-2 (MMP2) cleavable peptide having the amino acid sequence GPLGVR (SEQ ID NO: 118).

In some embodiments, the cleavable linker is a M2-specific peptide, having the amino acid sequence YEQDPWGVKWWY (SEQ ID NO: 116), or the amino acid sequence HLSWLPDVVYAW (SEQ ID NO: 117).

In some embodiments, the cleavable linker comprises a hypoxia inducible protein mediated cleavage site.

In some embodiments, the cleavable linker is a non-naturally occurring synthetic peptide, and comprises a protease cleavable site. In some embodiments, the cleavage site can be cleaved by a protease that is administered exogenously. In some embodiments, the cleavage site can be cleaved by a protease that is associated with a cancer targeted drug.

In some embodiments, the cleavable linker is a mutated peptide, where the mutated peptide contains a protease cleavable site, not occurring in the corresponding non-mutated peptide.

In some embodiments, the purpose of the instant program disclosed herein is generating therapeutic products for use in immunotherapy.

C. Engagers with Domains that Promotes Enhanced Phagocytic Activity and Immune Response of the Myeloid Cell

The tumor microenvironment (TME) constitutes an immunosuppressive environment. Influence of IL-10, glucocorticoid hormones, apoptotic cells, and immune complexes can interfere with innate immune cell function. Immune cells, including phagocytic cells settle into a tolerogenic phenotype. In macrophages, this phenotype, commonly known as the M2 phenotype is distinct from the M1 phenotype, where the macrophages are potent and capable of killing pathogens. Macrophages exposed to LPS or IFN-gamma, for example, can polarize towards an M1 phenotype, whereas macrophages exposed to IL-4 or IL-13 will polarize towards an M2 phenotype. LPS or IFN-gamma can interact with Toll-like receptor 4 (TLR4) on the surface of macrophages inducing the Trif and MyD88 pathways, inducing the activation of transcription factors IRF3, AP-1, and NFKB and thus activating TNF-□ genes, interferon genes, CXCL10, NOS2, IL-12, etc., which are necessary in a pro-inflammatory M1 macrophage response. Similarly, IL-4 and IL-13 bind to IL-4R, activation the Jak/Stat6 pathway, which regulates the expression of CCL17, ARG1, IRF4, IL-10, SOCS3, etc., which are genes associated with an anti-inflammatory response (M2 response). Expression of CD14, CD80, D206 and low expression of CD163 are indicators of macrophage polarization towards the M1 phenotype.

In some embodiments, the engagers comprise a binding domain that can bind to the extracellular domain of a receptor, such as a phagocytic receptor. Engagement with the monocyte or macrophage phagocytic receptor, for example, at a specific site may activate the receptor by enhancing the intracellular signaling mediated by the intracellular domain of the receptor. In some embodiments, the binding domain of the engager comprises a ligand for the phagocytic receptor. In some embodiments the binding domain binds to the ligand which then binds to the phagocytic receptor.

Some phagocytic receptors are more potent in activating phagocytosis than the others, and can induce rapid phagocytosis of the target cell. It is necessary to identify the potent phagocytic receptors. Most macrophage scavenger have broad binding specificity that may be used to discriminate between self and non-self in the nonspecific antibody-independent recognition of foreign substances. The type I and II class A scavenger receptors (SR-AI1 and SR-AII) are trimeric membrane glycoproteins with a small NH2-terminal intracellular domain, and an extracellular portion containing a short spacer domain, an a-helical coiled-coil domain, and a triple-helical collagenous domain. The type I receptor additionally contains a cysteine-rich COOH-terminal (SRCR) domain. These receptors are present in macrophages in diverse tissues throughout the body and exhibit an unusually broad ligand binding specificity. They bind a wide variety of polyanions, including chemically modified proteins, such as modified LDL, and they have been implicated in cholesterol deposition during atherogenesis. They may also play a role in cell adhesion processes in macrophage-associated host defense and inflammatory conditions.

Table 2A and Table 2B exemplify a non-extensive list of receptors or surface antigens associated with different myeloid cells, wherein the cells have a range of characteristics ranging from highly phagocytic to tolerogenic. Even within macrophages, some receptors are associated with the actively phagocytic M1 phenotype, while others are associated with the anti-inflammatory M2 phenotype which has dampened phagocytic response. Activation of the M1-associated receptors by engaging with an M1 receptor can generate a characteristic shift in the macrophage type, from M2 towards M1 phenotype.

Macrophage receptors that activate phagocytosis comprise an intracellular phagocytosis signaling domain that comprises a domain having one or more Immunoreceptor Tyrosine-based Activation Motif (ITAM) motifs. ITAMs are conserved sequences present in the cytoplasmic tails of several receptors of the immune system, such as T cell receptors, immunoglobulins (Ig) and FcRs. They have a conserved amino acid sequence motif consisting of paired YXXL/I motifs (Y=Tyrosine, L=Lysine and I=Isoleucine) separated by a defined interval (YXXL/I-X₆₋₈-YXXL/I). In addition, most ITAMs contain a negatively charged amino acid (D/E) in the +2 position relative to the first ITAM tyrosine. Phosphorylation of residues within the ITAM recruits several signaling molecules that activate phagocytosis. ITAM motifs are also present in the intracellular adapter protein, DNAX Activating Protein of 12 kDa (DAP12).

In some embodiments, the phagocytic signaling domain in the intracellular region can comprise a PI3kinase (PI3K) recruitment domain (also called PI3K binding domain). CD19, CD28, CSFR or PDGFR receptors comprise PI3 kinase recruitment to the binding domain. In some embodiments, the bi- or trispecific engager binds to a receptor such as any one or more of CD19, CD28, CSFR or PDGFR receptors. Engaging with such receptors lead to Akt mediated signaling cascade and activation of phagocytosis. The PI3K-Akt signaling pathway is important in phagocytosis, regulation of the inflammatory response, and other activities, including vesicle trafficking and cytoskeletal reorganization. The PI3kinase recruitment domain is an intracellular domain in a plasma membrane protein, which has tyrosine residues that can be phosphorylated, and which can in turn be recognized by the Src homology domain (SH2) domain of PI3Kp85. The SH2 domain of p85 recognizes the phosphorylated tyrosines on the cytosolic domain of the receptor. This causes an allosteric activation of p110 and the production of phosphatidylinositol-3,4,5-trisphosphate (PIP3) that is recognized by the enzymes Akt and the constitutively active 3′-phosphoinositide-dependent kinase 1 (PDK1) through their plekstrin homology domains. The interaction of Akt with PIP3 causes a change in the Akt conformation and phosphorylation of the residues Thr308 and Ser473 by PDK1 and rictor-mTOR complex, respectively. Phosphorylation of these two residues causes the activation of Akt which in turn phosphorylates, among other substrates, the enzyme glycogen synthase kinase-3 (GSK-3). GSK-3 has two isoforms, GSK-3a and GSK-3p both of which are constitutively active. The isoforms are structurally related but functionally nonredundant. Inactivation of GSK-3 is observed when the residues Ser21 in GSK-3a or Ser9 in GSK-3p, located in their regulatory N-terminal domains, are phosphorylated by Akt and other kinases. Inhibition of GSK-3 by phosphorylation is important for the modulation of the inflammation and in phagocytosis processes.

In some embodiments, a bi- or trispecific engager comprises a binding domain that binds to a receptor or part thereof that can activate pro-phagocytic signaling by engaging DAP12 activation.

In some embodiments, a bi- or trispecific engager comprises a binding domain that binds to a receptor or part thereof that promotes clustering of a group of receptors on a monocyte or macrophage or phagocytic cell, and potentiates phagocytosis. In some embodiments, clustering of receptors activate intracellular signaling pathways.

In some embodiments, the bi- or trispecific engager comprises a binding domain for Fc□R1 (CD89). Fc□R1 receptor engagement or cross-linking activates antigen mediated cytotoxicity, and activate phagocytosis on monocytes or macrophages. Fc□R1 is expressed constitutively in macrophages as well as some other cells such as neutrophils and eosinophils. It is especially advantageous in a trispecific engager when the engager comprises a binding domain that binds to an antigen on a target cell, such as a tumor cell, a binding domain specific for a monocyte or macrophage receptor such as CD206, and a binding domain for CD89. Given the length and flexibility of the design of the engager molecule, the CD206 and Fc□R1 (CD89) binding domains could engage and provide multiple activation signals by cross-linking with more than one phagocytosis specific receptors, and crosslinking with the target cell. Fc□R1 activation redirects monocytes or macrophages from M2 phenotype to killer M1 phenotype and can therefore having an Fc□R1 binding domain in a bi- or trispecific engager can be a powerful tool in repurposing tumor associated macrophages for tumor cytotoxicity.

In some embodiments, the bi- and trispecific engager comprises a binding domain that binds to a monocyte or macrophage scavenger receptor. There are currently eight classes of scavenger receptors (classes A-H). In some cases, multiple names have been assigned to the same receptor (e.g., MSR1, SR-AI, CD204, and SCARA1). In addition, there are proteins exhibiting scavenger receptor activity that have been named based on other criteria and have not been included in a general scavenger receptor nomenclature. Some examples include RAGE (SR-E1), LRP1, LRP2, ASGP, CD163, SR-PSOX, and CXCL16. In some embodiments, the bi- or trispecific engager comprises a binding domain that binds to a scavenger receptor, selected from lectin, dectin 1, mannose receptor (CD206), scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, CCR2, CX3CR1, CSF1R, Tie2, HuCRIg(L), and CD169 receptor. The binding domains bind to their respective ligands with a dissociation constant (K_{D}) of 10⁻⁵ to 10⁻¹² M or less, or, 10⁻⁷ to 10⁻¹² M or less or, 10⁻¹ to 10⁻¹² M (i.e. with an association constant (KA) of 10⁵ to 10¹² M or more, or, 10⁷ to 10¹² M or more or 10⁸ to 10¹² M).

In some embodiments, an exemplary binding domain of an engager that binds to the scavenger receptor SRA1 comprises a variable region having an amino acid sequence or a portion thereof, or a sequence having at least 95% sequence identity to a sequence:

(SEQ ID NO: 1)
EVQLVESGGGLVQAGGSLRLSCTASGRAVSTYAMGWFRQAPGKEREFVA
AMISSLSSKSYADTVKGRFTISRDYAKNTVYXQMNSLKPEDTADYYCAA
DLLPYSSSRSLPMGYDYWGQGTQVTVSS

Exemplary binding domains of an engager that binds to the scavenger receptor SRA1 can comprise a binding domain having an amino acid sequence of any one of SEQ ID NOs 2-7, or a portion thereof, or a sequence having at least 95% sequence identity to any one of the sequences:

(SEQ ID NO: 2)
EVQLVESGGGLVQAGGSLRLSCTASGRAVSTYAMGWFRQAPGKEREFVA
AMISSLSSKSYADSVKGRFTISRDYAKNTVYLQMNSLKPEDTADYYCAA
DLLPYSSTRSLPMGYDYWGQGTQVTVSS
(SEQ ID NO: 3)
EVQLVESGGGLVQAGGSLRLSCAASGSFSLYDMGWFSQAPGKEREFVAA
INWSGGSTAYADSVKGRFTISRDSAKNTVYLQMNSLKPEDTAVYYCAAK
PAKYHFGSGYRDFAEYPYWGQGTQVTVSS.
(SEQ ID NO: 4)
EVQLVESGGGLVQAGGSLRLSCAASGRTFSRYAMAWFRHAPGKDREFVA
AVSQSGLLTFYADSVKGRFTISRDNAKNTVYLQMNSLKPEDTAVYDCAA
XSRFPLVVPVAYENWGQGTQVTVSS; wherein X can be any
naturally occurring amino acid.
(SEQ ID NO: 5)
EVQLVESGGGLVQAGGSLRLSCAASGRTFSRYAMAWFRHAPGKDREFVA
AVSQSGLLTFYADSVKGRFTISRDNAKNTVYLQMNSLKPEDTAVYDCAA
DSRFPLVVPVAYENWGQGTQVTVSS.
(SEQ ID NO: 6)
EVQLVESGGGLVQVGGSLRLSCAASGISIRTHAMGWYRQAPGKQRELVA
TITSVTSGGSLNYADSVKGRFTISRDNAKNTVYLQMNSLKPEDTAVYYC
KLLGFDYRGQGTQVTVSS.
(SEQ ID NO: 7)
EVQLVESGGGLVQPGGSLRLSCAASGSIGRFVAMGWYRQAPGKQRELVA
TITSITSGGRTNYADSVKGRFTISRDNAKNTVYLQMNSLKPEDTAVYYC
NVVPYVNDYWGQGTQVTVSS.

In some embodiments, an exemplary binding domain of an engager that binds to the scavenger receptor RAGE can comprise a binding domain having an amino acid sequence of any one of SEQ ID NOs 8-15, or a portion thereof, or a sequence having at least 95% sequence identity to any one of the sequences.

(SEQ ID NO: 8)
EVQLVESGGGLVQAGDSLRLSCIASGRTFTMGWFRQAPGKEREFVAAIS
WSGGRTYYADSVKGRFTISRENAKNTVYLQMNSLKPEDTAVYCCATENL
ASSGSAYSDDRYNACGQGTQVTVSS
(SEQ ID NO: 9)
EVQLVESGGEVVQPGGSLRLSCAASGFTFDDRAIGWFRQAPGKEREGVA
CSANNDNRAFYEDSVKGRFAVSRDNAKNTVYLQMNSLKPEDTAVYYCAT
RCAAGRVNLYYGMDYVVGKGTLVTVSS
(SEQ ID NO: 10)
EVQLVESGGGLVQPGGSLRLSCAASGFTLGNYAIGWFRQAPGKEREGVS
CVDRDGGSTYYLDSVTGRFTTSRDDAENTVYLQMNSLIPDDTAVYYCAT
RLYGCSGYGRDYADWGQGTQVTVSS
(SEQ ID NO: 11)
EVQLVESGGGLVQAGGSLRLSCAVSGRTFSTDAFGWFRQAPGKEREFVS
AMRWNGSSSYYADLVKGRFTISRDNAKNTVYLLMNSLKPEDTAVYYCTA
GKRYGYYDYWGQGTQVTVSS
(SEQ ID NO: 12)
EVQLVESGGGLVQAGGSLRLSCAASGRTFSNYSMGWFRQAPGKEREFVA
TISWSGALTHYTDSVKGRFTISRDNAKNTVYLQMNSLKPEDTAVYYCAA
SDSDYGNKYDYWGQGTQVTVSS
(SEQ ID NO: 13)
EVQLVESGGGLVQAGGSLRLSCAASGRTVSDMTMGWFRQAPGKERVFVA
AISNSGLSTYYQDSVKGRFTISRDTANNTVALQMNSLKPEDTAVYFCAA
RSGWSGQYDYWGQGTQVTVSS
(SEQ ID NO: 14)
EVQLVESGGGLVQAGGSLRLSCAASGRIFNNYAMGWFRQAPGKEREFVA
GISWSGDSTLYADSVKGRFTTSRDNAKNTVYLQMNSLKPEDTANYYCAE
KQGADWAPYDYWGQGTQVTVSS
(SEQ ID NO: 15)
EVQLVESGGGLVQAGGSLRLSCVASELTFSLYRMGWFRQAPGKEREFVS
AMSTSGAGTYYADSVKGRFTISRDNPKNTVYLQMNSLKPEDTAVYYCVA
GVRFGVYDYWGQGTQVTVSS

In some embodiments, an exemplary binding domain of an engager that binds to the scavenger receptor Lox-1 can comprise a binding domain having an amino acid sequence of any one of SEQ ID NOs 16-26, or a portion thereof, or a sequence having at least 95% sequence identity to any one of the sequences.

(SEQ ID NO: 16)
EVQLVESGGGLVQPGGSLRLSCAASGFTLDDYAIGWFRQAPGKEREGVS
CISRTDGSTDYADSVKGRFTISRDNAKNTVYLQMNSLKPEDTAVYYCAA
GRTYYSGSYYFGLGSDEYDYWGQGTQVTVSS

Sequences of additional exemplary binding domains of an engager that binds to the scavenger receptor Lox-1 comprises a variable region having an amino acid sequence are given below:

(SEQ ID NO: 17)
EVQLVESGGGLVQPGGSLRLSCAASGSIFTINAMAWYRQAPGKQRELVA
HLTNSGRTGYADSVKGRFTISSDNAKNTVYLQMNSLKPEDTAVYYCNRL
GLHWSWGQGTQVTVSS
(SEQ ID NO: 18)
EVQLVESGGGLVQAGGSLRLSCAASIGTFSAYHMGWFRQAPGKERELVA
AISWSVSSTYYADSVKGRFTISRDNAKRTVSLQMDSLKPEDTAVYYCAA
RSGERYDYYKAQYEYWGQGTQVTVSS
(SEQ ID NO: 19)
EVQLVESGGGLVQPGGSLRLSCAAYGSFFSIGTMGWYRQPPGNQRELVA
VTYGLGSTNYAESVKGRFTISRDNAKNTVSLQMNSLKPEDTAVYYCYAE
IDTDPRSGEWDYWGQGTQVTVSS
(SEQ ID NO: 20)
EVQLVESGGGLVQPGGSLRLSCLPSTSTSSLRTVGWYRQGPGKQRDLVA
IMSAGTTRYADSVKGRFTISLDDAKNTVYLQMNSLKPEDTAVYICNGRP
VFSNVDYWGQGTQVTVSS
(SEQ ID NO: 21)
EVQLVESGGGLVQAGGSLRLSCAASGFTFDDYAIGWFRQAPGKEREGVS
CVSRDGGSTYYLDSVKGRFTISSDNAKNTVYLQMNSLKPEDAAVYYCAA
SRYDCSKYLIDYNYRGQGTQVTVSS
(SEQ ID NO: 22)
EVQLVKSGGGLVQAGGSLRLSCAASGRRFSTSGMGWFRQAPGREREFVX
GIXWNSRXTYYAESVKGRFTISRDNSKNTVYLQMNSLKPEDTAVYYCAT
NYYGSXWSVNSDDYDYWXQGXQVTVSS
(SEQ ID NO: 23)
EVQLVESGGGLVQAGGSLRLSCAASGRTFSNYAMGWFRQAPGKEREFVA
AITWSGSSTYYADSVKGRFTISRDNAKNTVYLQMNSLKPEDTAVYYCAA
AQRGRYYYLDRNVEYDYWGQGTQVTVSS
(SEQ ID NO: 24)
EVQLVESGGGLVQPGGSLRLSCAASGFTLDDYGIGWFRQAPGKEREGVS
CISSSDGSTDYADSVKGRFTISRDNAKNTVYLQMNNLKPEDTAVYYCAA
GRTYYSGSYYFGLGSDEYDYWGQGTQVTVSS
(SEQ ID NO: 25)
EVQLVESGGNLVQAGGSLRLSCAASGFTFDDYVIGWFRQAPGKEREGVS
CISSVEGSTYYADSVKGRFTISGDNAKNTVYLQMNSLKPEDTAVYYCAA
GTWLDCSGYGSYDMDYWGKGTLVTVSS
(SEQ ID NO: 26)
EVQLVESGGGLVQAGGSLRLSCAASGFTFDDYVIGWFRQAPGKEREGVS
CISSSEGSTYYAESVKGRFTISSDNAKNTVYLQMNSLKPEDTAVYYCAA
STWLDFVHGNEYDYRGQGTQVTVSS

In some embodiments, an exemplary SRA-1 binding CDR1 sequence can be any one of the amino acid sequences: TYAMG (SEQ ID NO: 31), YDMG (SEQ ID NO: 32), RYAMA (SEQ ID NO: 33), THAMG (SEQ ID NO: 34), FVAMG (SEQ ID NO: 35).

In some embodiments, an exemplary SRA-1 binding CDR2 sequence can be any one of the amino acid sequence;

(SEQ ID NO: 36)
AMISSLSSKSYADTVKG
(SEQ ID NO: 37)
AMISSLSSKSYADSVKG
(SEQ ID NO: 38)
AINWSGGSTAYADSVKG
(SEQ ID NO: 39)
AVSQSGLLTFYADSVKG
(SEQ ID NO: 40)
AVSQSGLLTFYADSVKG
(SEQ ID NO: 41)
TITSVTSGGSLNYADSVKG
(SEQ ID NO: 42)
TITSITSGGRTNYADSVKG

In some embodiments, an exemplary SRA-1 binding CDR3 sequence sequences can be any one of the amino acid sequences:

(SEQ ID NO: 43)
DLLPYSSSRSLPMGYD
(SEQ ID NO: 44)
DLLPYSSTRSLPMGYDY
(SEQ ID NO: 45)
KPAKYHFGSGYRDFAE
(SEQ ID NO: 46)
XSRFPLVVPVAYEN
(SEQ ID NO: 47)
DSRFPLVVPVAYEN
(SEQ ID NO: 48)
LGFDY
(SEQ ID NO: 49)
VPYVNDY

wherein, X is a naturally occurring amino acid.

In some embodiments, an exemplary RAGE binding CDR1 sequence can be any one of the amino acid sequences: DRAIG (SEQ ID NO: 50), NYAIG (SEQ ID NO: 51), TDAFG (SEQ ID NO: 52), NYSMG (SEQ ID NO: 53), DMTMG (SEQ ID NO: 54), NYAMG (SEQ ID NO: 55), or LYRMG (SEQ ID NO: 56).

In some embodiments, an exemplary RAGE binding CDR2 sequence can be any one of the amino acid sequences:

(SEQ ID NO: 57)
AISWSGGRTYYADSVKG
(SEQ ID NO: 58)
CSANNDNRAFYEDSVKG
(SEQ ID NO: 59)
CVDRDGGSTYYLDSVTG
(SEQ ID NO: 60)
AMRWNGSSSYYADLVKG
(SEQ ID NO: 61)
TISWSGALTHYTDSVKG
(SEQ ID NO: 62)
AISNSGLSTYYQDSVKG
(SEQ ID NO: 63)
GISWSGDSTLYADSVKG
(SEQ ID NO: 64)
AMSTSGAGTYYADSVKG
(SEQ ID NO: 65)
CISRTDGSTDYADSVKG

In some embodiments, an exemplary RAGE binding CDR3 sequence can be any one of the amino acid sequences:

(SEQ ID NO: 66)
ENLASSGSAYSDDRYN
(SEQ ID NO: 67)
RCAAGRVNLYYGMDY
(SEQ ID NO: 68)
RLYGCSGYGRDYAD
(SEQ ID NO: 69)
GKRYGYYDY
(SEQ ID NO: 70)
SDSDYGNKYDY
(SEQ ID NO: 71)
RSGWSGQYDY
(SEQ ID NO: 72)
KQGADWAPYDY
(SEQ ID NO: 73)
GVRFGVYDY

In some embodiments, Lox-1 binding CDR1 sequences can be any one of the amino acid sequences: DYAIG (SEQ ID NO: 74), INAMA (SEQ ID NO: 75), AYHMG (SEQ ID NO: 76), IGTMG (SEQ ID NO: 77), LRTVG (SEQ ID NO: 78), DYAIG (SEQ ID NO: 79), TSGMG (SEQ ID NO: 80), NYAMG (SEQ ID NO: 81), DYGIG (SEQ ID NO: 82), DYVIG (SEQ ID NO: 83).

In some embodiments, Lox-1 binding CDR2 sequences can be any one of the amino acids sequences:

(SEQ ID NO: 84)
HLTNSGRTGYADSVKG
(SEQ ID NO: 85)
AISWSVSSTYYADSVKG
(SEQ ID NO: 86)
VTYGLGSTNYAESVKG
(SEQ ID NO: 87)
IMSAGTTRYADSVKG
(SEQ ID NO: 88)
CVSRDGGSTYYLDSVKG
(SEQ ID NO: 89)
GIXWNSRXTYYAESVKG
(SEQ ID NO: 90)
AITWSGSSTYYADSVKG
(SEQ ID NO: 91)
CISSSDGSTDYADSVKG
(SEQ ID NO: 92)
CISSVEGSTYYADSVKG
(SEQ ID NO: 93)
CISSSEGSTYYAESVKG

wherein, X is a naturally occurring amino acid.

In some embodiments, Lox-1 binding CDR3 sequences can be any one of the amino acid sequences:

(SEQ ID NO: 94)
GRTYYSGSYYFGLGSD
(SEQ ID NO: 95)
LGLHWS
(SEQ ID NO: 96)
RSGERYDYYKAQYEY
(SEQ ID NO: 97)
EIDTDPRSGEWDY
(SEQ ID NO: 98)
RPVFSNVDY
(SEQ ID NO: 99)
SRYDCSKYLIDYNY
(SEQ ID NO: 100)
NYYGSXWSVNSDDYDY
(SEQ ID NO: 101)
AQRGRYYYLDRNVEYD
(SEQ ID NO: 102)
GRTYYSGSYYFGLGSDEYDY
(SEQ ID NO: 103)
GTWLDCSGYGSYDMDY
(SEQ ID NO: 104)
STWLDFVHGNEYDY

In some embodiments, the bi- or trispecific engager comprises a binding domain that binds to a protein that can generate phagocytosis activation signals or pro-inflammatory signals, for example via activation of any one of: MRC1, ItgB5, MERTK, ELMO, BAIL Tyro3, Axl, Traf6, Syk, MyD88, Zap70, PI3K, FcγR1, FcγR2A, FcγR2B2, FcγR2C, FcγR3A, FcER1, FcaRl, BAFF-R, DAP12, NFAM1, and CD79b.

In some embodiments, the bi- or trispecific engager comprises a binding domain that binds to the extracellular domain of a TREM protein. TREM 1, 2, 3. TREMs share common structural properties, including the presence of a single extracellular immunoglobulin-like domain of the V-type, a trans-membrane domain and a short cytoplasmic tail. In particular, the TREM trans-membrane domain (TM) possesses negatively charged residues that interact with the positively charged residues of the DNAX Activating Protein of 12 kDa (DAP12), a trans-membrane adaptor containing an immunoreceptor tyrosine-based activation motif (ITAM).

D. Engagers with Domains that Promote Inflammatory Activity of the Myeloid Cell

Activation of monocytes or macrophages can lead to increase in inflammatory activity. Activated M1 monocytes or macrophages are characterized by IFN-gamma production, as well as production of pro-inflammatory cytokines, IL-1b, IL-6, CSF, GMCSF, and TNF to name a few. In some embodiments, a monocyte or macrophage M1 phenotype is associated with potent pro-inflammatory response associated with IL-1 signaling cascade and inflammasome activation.

In some embodiments, a bi- or trispecific engager may comprise a domain that generates a signal is necessary to trigger inflammasomes and pro-inflammatory signals. Toll-like receptors, TLRs are known to induce inflammasome activation. TLRs elicit conserved inflammatory pathways culminating in the activation of NF-κB and activating protein-1 (AP-1). TLR ligands include high-mobility group B1 (HMGB1), heat shock proteins (HSP60, HSP70), endotoxins, and extracellular matrix components. TLR2 and TLR4, for example comprise extracellular domains which are activated by ligand binding, and which is turn activates a pro-inflammatory cascade associated with inflammasome activation. Intracellular signaling pathway is mediated by signaling proteins e.g., Nod-like receptors (NLRs) that recruit proinflammatory caspases and induce their cleavage and activation. This leads to direct activation of ROS, and often results in a violent cell death known as pyroptosis. There are four inflammasome complexes, NLRP1m, NLRP3, IPAF and AIM2.

In some embodiments, a bi- or trispecific engager may comprise a binding domain that generates a signal is necessary to trigger inflammasomes and pro-inflammatory signal binds to TLRs, such as TLR4. TLR4 is expressed in monocytes or macrophages and is induced by LPS and other ligands. In some embodiments, a bi- or trispecific engager may bind to a TLR ligand which then binds to the TLR.

E. Engagers with Domains that Promote Cell Adhesion and Inflammatory Activity of the Myeloid Cell

Cell-cell and cell-substratum adhesion is mediated by the binding of integrin extracellular domains to diverse protein ligands; however, cellular control of these adhesive interactions and their translation into dynamic cellular responses, such as cell spreading or migration, requires the integrin cytoplasmic tails. These short tails bind to intracellular ligands that connect the receptors to signaling pathways and cytoskeletal networks. Integrins are heterodimeric adhesion receptors formed by the non-covalent association of α and β subunits. Each subunit is a type I transmembrane glycoprotein that has relatively large extracellular domains and, with the exception of the β4 subunit, a short cytoplasmic tail. Individual integrin family members have the ability to recognize multiple ligands. Integrins can bind to a large number of extracellular matrix proteins (bone matrix proteins, collagens, fibronectins, fibrinogen, laminins, thrombospondins, vitronectin, and von Willebrand factor), reflecting the primary function of integrins in cell adhesion to extracellular matrices. Many “counter-receptors” are ligands, reflecting the role of integrins in mediating cell-cell interactions. Integrins undergo conformational changes to increase ligand affinity.

The Integrin β₂ subfamily consists of four different integrin receptors, α_{M}β₂ (CD11b/CD18, Mac-1, CR3, Mo-1), α_{L}β₂ (CD11a/CD18, LFA-1), α_{X}β₂ (CD11c/CD18), and α_{D}β₂ (CD11d/CD18). These leukocyte integrins are involved in virtually every aspect of leukocyte function, including the immune response, adhesion to and transmigration through the endothelium, phagocytosis of pathogens, and leukocyte activation.

The a subunits of all β₂ integrins contain an inserted region of ˜200 amino acids, termed the I or A domain. Highly conserved I domains are found in several other integrin a subunits and other proteins, such as certain coagulation and complement proteins. I domains mediate protein-protein interactions, and in integrins, they are integrally involved in the binding of protein ligands. Although the I domains dominate the ligand binding functions of their integrins, other regions of the a subunits do influence ligand recognition. As examples, in α_{M}β₂ a mAb (OKM1) recognizing an epitope outside the I domain but in the QM subunit inhibits ligand binding; and the EF-hand regions in α_{L}β₂ and α₂β₁, integrins with I domains in their a subunits, contribute to ligand recognition. The α_{M} subunit, and perhaps other α subunits, contains a lectin-like domain, which is involved in engagement of non-protein ligands, and occupancy may modulate the function of the I domain.

As integrins lack enzymatic activity, signaling is instead induced by the assembly of signaling complexes on the cytoplasmic face of the plasma membrane. Formation of these complexes is achieved in two ways; first, by receptor clustering, which increases the avidity of molecular interactions thereby increasing the on-rate of binding of effector molecules, and second, by induction of conformational changes in receptors that creates or exposes effector binding sites. Within the ECM, integrins have the ability to bind fibronectin, laminins, collagens, tenascin, vitronectin and thrombospondin. Clusters of integrin/ECM interactions form focal adhesions, concentrating cytoskeletal components and signaling molecules within the cell. The cytoplasmic tail of integrins serve as a binding site for α-actinin and talin which then recruit vinculin, a protein involved in anchoring F-actin to the membrane. Talin is activated by kinases such as protein kinase C (PKC□).

Integrins are activated by selectins. Leucocytes express L-selectin, activated platelets express P-selectin, and activated endothelial cells express E- and P-selectin. P-selectin-mediated adhesion enables chemokine- or platelet-activating factor-triggered activation of β2 integrins, which stabilizes adhesion. It also facilitates release of chemokines from adherent leucocytes. The cytoplasmic domain of P-selectin glycoprotein ligand 1 formed a constitutive complex with Nef-associated factor 1. After binding of P-selectin, Src kinases phosphorylated Nef-associated factor 1, which recruit the phosphoinositide-3-OH kinase p85-p110δ heterodimer and result in activation of leukocyte integrins. E-selectin ligands transduce signals that also affect β2 integrin function. Selectins trigger activation of Src family kinases. SFKs activated by selectin engagement phosphorylate the immunoreceptor tyrosine-based activation motifs (ITAMs) in the cytoplasmic domains of DAP12 and FcRγ. In some respects, CD44 is sufficient to transduce signals from E-selectin. CD44 triggers the inside-out signaling of integrins. A final common step in integrin activation is binding of talin to the cytoplasmic tail of the β subunit. Kindlins, another group of cytoplasmic adaptors, bind to a different region of integrin β tails. Kindlins increase the clustering of talin-activated integrins. Kindlins are responsive to selectin signaling, however, kindlins are found mostly in hematopoietic cells, such as neutrophils. Selectin signaling as well as signaling upon integrin activation by chemokines components have shared components, including SFKs, Syk, and SLP-76.

In some embodiments, the engagers comprise a binding domain that can bind to the extracellular domain of an adhesion molecule such as an integrin or a selectin, for example, a P-selectin, L-selectin or E-selectin.

F. Engagers with Binding Domains that Inhibits Anti-Phagocytic and Anti-Inflammatory Activity of the Myeloid Cell

In one aspect, a bi- or a trispecific engager may comprise an additional functional domain that inhibits CD47 mediated downregulation of monocyte or macrophage phagocytosis. Tumor cells typically express the “don't eat me” signal CD47 that binds to a monocyte or macrophage receptor SIRP-α, and inhibits phagocytosis. Inhibition of CD47 therefore counteracts the tumor cell mediated anti-phagocytosis activity. One arm of a bi- or trispecific engager may comprise a CD47 blocker. The CD47 blocker associated with the engager may be the extracellular CD47-binding domain of SIRP-α, acting as a decoy receptor or neutralizing receptor.

In one aspect, disclosed herein are compositions that can inhibit phagocytosis regulatory signal transduction by members of the Siglec family of membrane proteins that are expressed on immune cells. Various members of the family transduce checkpoint signal upon contact with sialylated glycans on membrane proteins. In some members, the intracellular domains of the Siglec proteins comprise multiple immunoreceptor tyrosine-based inhibitory motifs (ITIMs). ITIMs share a consensus amino acid sequence in their cytoplasmic tail, namely (I/V/L/S)—X—Y—X—X-(L/V), where X denotes any amino acid, I=Isoleucine, V=valine, L=Lysine, S=Serine, Y=Tyrosine. Phosphorylation of the Tyrosine residues at the ITIM motif recruit either of two SH2 domain-containing negative regulators: the inositol phosphatase SHIP (Src homology 2-containing inositol polyphosphate 5-phosphatase) or the tyrosine phosphatase SHP-1 (Src homology 2-containing protein tyrosine phosphatase-1). A leucine in the (Y+2) position favors binding to SHIP, whereas an isoleucine in the (Y-2) position favors SHP-1 binding. ITIMs can also bind to another tyrosine phosphatase, SHP-2, but evidence for SHP-2 playing a functional role in ITIM-mediated inhibition is less clear than for the other mediators. Therefore, activation of the Siglec membrane proteins at the extracellular ligand binding domain by binding with a sialic acid residue, (e.g. in sialylated membrane glycan proteins), the ITIMs receive the intracellular signals, which are phosphorylated, and initiate the SHP mediated signaling for immunomodulation, including reduction in phagocytic potential.

Siglec family receptors comprise the membrane proteins, siglec 1 (CD169), siglec 2 (CD22), siglec 3 (CD33), siglec 4 (MAG), siglec 5, siglec 6, siglec 7, siglec 8, siglec 9, siglec 10, siglec 11, siglec 12, siglec 13, siglec 14, siglec 15, siglec 16.

In some embodiments the composition described herein may comprise a binding domain for a Siglec receptor (SgR) such that the SgR receptor is blocked, and SgR induced immunoregulatory intracellular signaling is inhibited.

### Specific Multimerization Domains for Engagers

Also envisioned in the molecular design of bi- and trispecific engagers are additional structures and helpers that assist in the engager's capability to modularly and concomitantly engage with multiple targets. These designs include additional anchoring or clasping elements for two or more binding domains separated by linkers, such as in a bi- or trispecific antibody, a tribody or a triple body formats. These higher order multi-specific binding domains often require inclusion of the multimerization domains to improve stability and flexibility in binding the multiple domains on the same and different cells. The additional anchoring or clasping elements occur in cognate pairs, such that one of the cognate pair of the anchoring or clasping modality is attached to one of the binding domains of an engager and the other of the pair is attached to the other of the cognate pair.

In some embodiments, the engager is a recombinant protein comprising multiple binding domains as described throughout the specification, each having individual binding specificities, that are each linked together by linkers having cognate peptide anchoring or clasping elements that exhibit complementary binding with each other. For example, one binding domain of the recombinant protein is fused with the first of a pair of cognate peptides, and the other binding domain is fused with the second of the pair of peptides, wherein, the pair of peptides exhibit complementary binding with each other, wherein the pair of cognate peptides comprise: (a) leucine zipper domains that exhibit complementary binding with each other; for example, leucine zippers in naturally occurring protein-protein interactions, such as the zipper sequences within the binding regions of c-Fos and c-Jun proteins, (b) synthetic peptides designed to specifically bind to each other via synthetic clasps.

In some embodiments, the therapeutic agent is a recombinant protein comprising multiple binding fragments configured to facilitate accelerated association with each other by means of leucine zipper peptide pairs comprised in the recombinant proteins. Leucine zipper sequences often comprise a heptad leucine repeat and constitute adhesive peptide pairs when two peptides possess the leucine zipper structures. Among the naturally occurring leucine zippers, the c-Fos and c-June pairs are most widely known. They exhibit a strong binding affinity with K_{D}: 5.4×10⁻⁸ M. They form parallel coils. In some embodiments, the leucine zipper coil is the coil of the c-Fos: c-Jun pair. In some embodiments, exemplary cognate pair anchoring or clasping elements include the ACID-p1 (LZA) and BASE-p1 (LZB) pair; which are prevented from homodimerizing because of the electrostatic repulsion between the charges among the amino acid side chains. Prevention of homodimerization can be beneficial in a number of embodiments.

Exemplary c-Fos leucine zipper domain comprises an amino acid sequence as follows:

(SEQ ID NO: 119)
IARLEEKVKTLKAQNSELASTANMLREQVAQLKQKVMNH

Exemplary c-Jun leucine zipper domain comprises an amino acid sequence as follows:

(SEQ ID NO: 120)
TDTLQAETDQLEDEKSALQTEIANLLKEKEKLEFILAAH

Exemplary LZA leucine zipper domain comprises an amino acid sequence as follows:

(SEQ ID NO: 121)
AQLEKELQALEKENAQLEWELQALEKELAQK

Exemplary LZB leucine zipper domain comprises an amino acid sequence as follows:

(SEQ ID NO: 122)
AQLKKKLQALKKKNAQLKWKLQALKKKLAQK

In some embodiments, the therapeutic agent is a recombinant protein comprising multiple binding fragments configured to facilitate accelerated association with each other by means of c-Fos/c-Jun binding domains in the peptide pairs comprised within the recombinant proteins.

In some embodiments, the anchoring or clasping elements exhibit specific heterodimerizing capabilities and do not exhibit homodimerization.

In some embodiments, the therapeutic agent is a recombinant protein comprising multiple binding fragments configured to facilitate accelerated association with each other by means of synthetic clasps. In some embodiments, the synthetic anchoring or clasping elements are designed to heterodimerize and prevent homodimerization.

In some embodiments the synthetic clasps of the linkers are non-peptide crosslinkers.

In some embodiments the complementary binding of cognate peptides with each other can be via chemical binding, such as crosslinking. Chemical crosslinkers can be useful for activating the crosslinking in vitro. There are homo- and heterobifunctional protein crosslinkers that can be commercially available. Examples include BS2G crosslinker (BS²G; Bis[Sulfosuccinimidyl] glutarate) is an amine-reactive, water soluble, homobifunctional protein crosslinker (both binding units at the opposite ends of a spacer arm have the identical reactive groups), or its membrane permeable version, DSG (Disuccinimidyl glutarate; Di(N-succinimidyl) glutarate); BS3 crosslinker (Bis[sulfosuccinimidyl] suberate; Sulfo-DSS; BSSS) or DST crosslinker (Disuccinimidyl tartrate), are among other homobifunctional crosslinkers for peptides; whereas BMPS (N-(β-Maleimidopropyloxy) succinimide ester; MBS crosslinker (m-Maleimidobenzoyl-N-hydroxysuccinimide ester); PDPH crosslinker (3-[2-Pyridyldithio]propionyl hydrazide) provide examples of some heterobifunctional crosslinkers.

In some embodiments, the variable light chain (V_{L}) subunit and the variable heavy chain (V_{H}) regions arranged in tandem within a multi-specific engager may be linked via two linkers having the cognate peptide anchoring or clasping elements. The length of the linkers can limit or facilitate specific V_{L}-V_{H} associations. For example, limiting the linker peptide length to less than 10 amino acids restricts the association between two adjacent V_{L} and V_{H} domains.

In some embodiments, the anchoring or clasping elements exhibit an affinity having a K_{D}: less than 5×10⁻⁶M, or less than 10⁻⁶M, less than 5×10⁻⁷M, or less than 4×10⁻⁷M, or less than 3×10⁻⁷M, or less than 2×10⁻⁷M; or less than 10⁻⁷M, or less than 9×10⁻⁸M, or less than 8×10⁻⁸M, or less than 7×10⁻⁸M, or less than 6×10⁻⁸M, or less than 5×10⁻⁸M, or less than 4×10⁻⁸M, or less than 3×10⁻⁸M, or less than 2×10⁻⁸M, or less than 10⁻⁸M, or less than 10⁻⁸M, or less than 10⁻⁰M, or higher affinity.

Additionally, inclusion of the additional anchoring or hetero-multimerization domains in these higher order multi-specific engagers (e.g., engagers with multiple binding domains) that are formed by the assembly of heterodimeric or heteromultimeric units assist in the production, folding, stability and tissue availability of the multi-specific engagers.

### Co-Expression of an Inflammatory Gene

In one aspect, the recombinant nucleic acid comprises a coding sequence for a pro-inflammatory gene, which is expressed in an engineered cell. In some embodiments, the pro-inflammatory gene is a cytokine. Examples include but not limited to TNF-α, IL-1α, IL-1β, IL-6, CSF, GMCSF, or IL-12 or interferons. In some embodiments, the recombinant nucleic acid encoding a coding sequence of a proinflammatory gene is a therapeutic agent, such as an additional therapeutic agent to accompany at least the first therapeutic agent.

### Peptide Linker

In some embodiments, the extracellular antigen binding domains, scFvs or binding domains are linked with each other by a linker. In some embodiments, where there are more than one scFv at the extracellular antigen binding domain the more than scFvs are linked with each other by linkers.

In some embodiments the linkers are flexible. In some embodiments the linkers comprise a hinge region. Linkers are usually short peptide sequences. In some embodiments the linkers are stretches of Glycine and one or more Serine residues. Other amino acids preferred for a peptide linker include but are not limited to threonine (Thr), serine (Ser), proline (Pro), glycine (Gly), aspartic acid (Asp), lysine (Lys), glutamine (Gln), asparagine (Asn), and alanine (Ala) arginine (Arg), phenylalanine (Phe), glutamic acid (Glu). Of these Pro, Thr, and Gln are frequently used amino acids for natural linkers. Pro is a unique amino acid with a cyclic side chain which causes a very restricted conformation. Pro-rich sequences are used as interdomain linkers, including the linker between the lipoyl and E3 binding domain in pyruvate dehydrogenase (GA₂PA₃PAKQEA₃PAPA₂KAEAPA₃PA₂KA). For the purpose of the disclosure, the empirical linkers may be flexible linkers, rigid linkers, and cleavable linkers. Sequences such as (G4S)x (where x is multiple copies of the moiety, designated as 1, 2, 3, 4, and so on) comprise a flexible linker sequence. Other flexible sequences used herein include several repeats of glycine, e.g., (Gly)6 or (Gly)8. On the other hand, a rigid linker may be used, for example, a linker (EAAAK)x, where x is an integer, 1, 2, 3, 4 etc. gives rise to a rigid linker.

The length of a linker peptide can be crucial in the design of a multi-specific engager. For example, limiting the linker peptide length to less than 10 amino acids restricts the association between two adjacent domains. In some embodiments, the linker may comprise a anchoring or clasping function and may comprise a crosslinking moiety. The cross linking moiety may be a peptide or a chemical cross linking moiety, several of which are described in the previous section.

Specific peptides with specific functions have been discussed elsewhere in the document. In some embodiments, a peptide linker may further function as an activator or a signal in a myeloid cell. For example, a TLR4 activation peptide may be incorporated within the linker between two binding domains of an engager, and the TLR4 activation peptide binds to and activates a TLR4 signal in a monocyte or macrophage.

In some embodiments, a peptide linker may further function as a conditionally cleavable linker. By conditionally cleavable it is understood that the peptide is cleaved when the agent that cleaves it is available. For example, an MMP2 cleavable peptide is described herein, which is readily cleaved only when the peptide is in a region rich in MMP2. An exemplary MMP2 cleavable peptide is GPLGVR.

In some embodiments, a peptide linker may further function as a targeting peptide. For example, an M2 peptide is described, which is can bind to a M2 monocyte or macrophage, which is the predominant tumor associated monocyte or macrophage phenotype. An exemplary peptide is YEQDPWGVKWWY.

Any one or more peptide linkers may comprise specialized functions, such as they can dimerize, trimerize or multimerize. In some embodiments, one or more linkers may comprise leucine zipper sequences.

In some embodiments, the peptide linker is 5, 6, 7, 8, 9, 10, 11, 12, 13, 14, 15, 16, 17, 18, 19 or 20 amino acids in length. In some embodiments, a peptide linker or the two linker peptides with an anchor or a clasp together span a length of 50 amino acids or less, 45 amino acids or less, 40 amino acids or less, 35 amino acids or less, 30 amino acids or less, 25 amino acids or less, 20 amino acids or less, 15 amino acids or less, 10 amino acids or less, or 5 amino acids or less. In some embodiments, a peptide linker or the two linker peptides with an anchor or a clasp together span a length of 25 amino acids or less, 24 amino acids or less, 23 amino acids or less, 22 amino acids or less, 21 amino acids or less, 20 amino acids or less, 19 amino acids or less, 19 amino acids or less, 18 amino acids or less, 17 amino acids or less, 16 amino acids or less, 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, or 5 amino acids or less.

### Methods for Preparing Monocyte or Macrophage Specific Engagers

The engagers described herein are produced as recombinant proteins. Generally, a polynucleotide sequence is constructed that encodes the recombinant protein is prepared and inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

The recombinant polynucleotide is synthesized by ligating DNA encoding, for example, a first binding domain, a linker, and a second binding domain in the same open reading frame using the molecular cloning techniques well known to one of skill in the art. In some embodiments, one or more polynucleotide sequences are arranged under the same promoter and regulatory elements for generation of a single polypeptide. In some embodiments, a short spacer may be inserted between two adjacent polynucleotides encoding two polypeptides wherein the spacer may encode a post translational cleavage site. The two polypeptides can be separated after translation by induction of the cleavage at the specific cleavage site. In some embodiments, the construct may be monocistronic or polycistronic. In some embodiments, more than one polypeptides are generated which then reassemble after translation. For example, light chain and heavy chain domains of an antibody or parts thereof can be generated by translation from two independent polynucleotide sequences, which are allowed to freely assemble with each other post-translationally. Alternatively, multiple polypeptide chains containing LC and HC variable domains that bind with each other are transcribed and translated from a single polynucleotide, which is cleaved after translation into respective peptide chains which can then reassemble. The polypeptide having a leader sequence is a preprotein and can have the leader sequence cleaved by the host cell to form the mature form of the polypeptide.

In some embodiments, the polynucleotide construct encodes an N-terminal signal sequence upstream of the polypeptide for secretion of the polypeptides. In some embodiments, the N terminal signal sequence comprises a secretion sequence. The resulting translated protein product having the N-terminal signal sequence for secretion would be secreted by the cell.

In some embodiments the plasmid vector is introduced or incorporated in the cell by known methods of transfection, such as using lipofectamine, or calcium phosphate, or via physical means such as electroporation or nucleofection. In some embodiments the viral vector is introduced or incorporated in the cell by infection, a process commonly known as viral transduction.

In some embodiments, recombinant nucleic acid is integrated or incorporated in an expression vector. A vector comprises one or more promoters, and other regulatory components, including enhancer binding sequence, initiation and terminal codons, a 5′UTR, a 3′UTR comprising a transcript stabilization element, optional conserved regulatory protein binding sequences and others.

In some embodiments the vectors of use in the application are specifically enhanced for expression. Other exemplary vectors of use throughout the process include phages, cosmids, or artificial chromosomes.

It is understood that any one of the first binder domains (domain binding to a target cell such as a cancer cell or a diseased cell or a pathogen) can be designed in combination with a second binder domain that binds to a myeloid cell or a third binding domain described anywhere in the specification.

Viral Vectors: In some embodiments, the vector for expression of the recombinant protein is of a viral origin, namely a lentiviral vector or an adenoviral vector. In some embodiments, the nucleic acid encoding the recombinant nucleic acid is encoded by a lentiviral vector. In some embodiments the lentiviral vector is prepared in-house and manufactured in large scale for the purpose. In some embodiments, commercially available lentiviral vectors are utilized, as is known to one of skill in the art.

In some embodiments the viral vector is an Adeno-Associated Virus (AAV) vector.

Lipid nanoparticle mediated delivery: Lipid nanoparticles (LNP) may comprise a polar and or a nonpolar lipid. In some embodiments cholesterol is present in the LNPs for efficient delivery. LNPs are 100-300 nm in diameter provide efficient means of mRNA delivery to various cell types, including monocytes or macrophages. In some embodiments, LNP may be used to introduce the recombinant nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an mRNA molecule. In some embodiments, the LNP encapsulates the nucleic acid wherein the nucleic acid is inserted in a vector, such as a plasmid vector. In some embodiments, the LNP encapsulates the nucleic acid wherein the nucleic acid is a circRNA molecule.

In some embodiments, the LNP is used to deliver the nucleic acid into a subject. LNP can be used to deliver nucleic acid systemically in a subject. It can be delivered by injection. In some embodiments, the LNP comprising the nucleic acid is injected by intravenous route. In some embodiments the LNP is injected subcutaneously.

Microbubble mediated delivery: In some embodiments, microbubbles can be used for delivery of a composition comprising e.g., a nucleic acid in a subject. Perfluorocarbon-filled microbubbles are stable for circulating in the vasculature as blood pool agents, they act as carriers of these agents until the site of interest is reached. Ultrasound applied over the skin surface can then be used to burst the microbubbles at this site, causing localized release of the drug. Various other forms of microbubbles include Sonazoid Optison, gas-filled albumin microbubble, and PESDA. Optimization of the composition of the microbubble with respect to the composition of the therapeutic agent that is delivered, along with the site of delivery intended is necessary.

In some embodiments, the recombinant proteins, for example the engagers, or the inflammatory proteins that are co-expressed, or any associated protein designed to be expressed in a myeloid cell may be encoded by a recombinant nucleic acid, wherein the recombinant nucleic acid is an RNA. In some embodiments, the recombinant nucleic acid is an mRNA. In some embodiments, the mRNA comprises one or more modifications for enhanced expression and stability. In some embodiments, the mRNA may be circularized. In some embodiments, the modifications may include but are not limited to: replacement of a nucleobase with a base analog, or a modified nucleotide; inserting one or more motifs within the mRNA, and introducing modifications in the 5′- and 3′ UTRs. In some embodiments, the recombinant nucleic acid may be administered directly in a subject in need thereof.

### Pharmaceutical Composition

Provided herein is a pharmaceutical composition, comprising at least a first therapeutic agent which comprises monocyte or macrophage specific engagers. The monocyte or macrophage specific engagers in the composition may be in the form of peptides or polypeptides or a complex of multiple peptides. The monocyte or macrophage specific engagers may be provided in a composition as purified recombinant proteins. The monocyte or macrophage specific engagers may be provided in a composition as conjugated recombinant proteins, V_{HH} complexes, scFv complexes or nanobodies. The monocyte or macrophage specific engagers may be in the form of a polynucleotide encoding the recombinant monocyte or macrophage specific engagers. In some embodiments, polynucleotide encoding the monocyte or macrophage specific engagers may comprise DNA, mRNA or circRNA or a liposomal composition of any one of these. The liposome is a LNP.

Pharmaceutical compositions can include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Acceptable carriers are physiologically acceptable to the administered patient and retain the therapeutic properties of the compounds with/in which it is administered. Acceptable carriers and their formulations are generally described in, for example, Remington' pharmaceutical Sciences (18^{th} ed. A. Gennaro, Mack Publishing Co., Easton, Pa. 1990). One example of carrier is physiological saline. A pharmaceutically acceptable carrier is a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. Nor should an acceptable carrier alter the specific activity of the neoantigens.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (i.e., systemic or local). Thus, compositions include carriers, diluents, or excipients suitable for administration by various routes.

In some embodiments, a composition can further comprise an acceptable additive in order to improve the stability of immune cells in the composition. Acceptable additives may not alter the specific activity of the immune cells. Examples of acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

The pharmaceutical composition can be administered, for example, by injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride can be included in the composition. The resulting solutions can be packaged for use as is, or lyophilized; the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution thereof.

Compositions can be conventionally administered intravenously, such as by injection of a unit dose, for example. For injection, an active ingredient can be in the form of a parenterally acceptable aqueous solution which is substantially pyrogen-free and has suitable pH, isotonicity and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Additionally, compositions can be administered via aerosolization.

When the compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available kits.

Acceptable carriers can contain a compound that stabilizes, increases or delays absorption, or increases or delays clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (e.g., Fix (1996) Pharm Res. 13:1760 1764; Samanen (1996) J. Pharm. Pharmacol. 48:119 135; and U.S. Pat. No. 5,391,377).

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations in the blood are contemplated.

### Treatment Methods

The instant disclosure comprises methods of treatment for diseases such as cancer, and infection, where enhanced phagocytosis by myeloid cells can be beneficial to remove diseased cells, or infected cells.

Cancers include, but are not limited to T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, a composition comprising at least a first therapeutic agent, comprising a monocyte or macrophage specific engager is administered per administration dose. In some embodiments, a composition the first therapeutic agent is administered in combination with a second or a third therapy.

In some embodiments, the composition comprising at least a first therapeutic agent comprising a monocyte or macrophage specific engager is administered once. In some embodiments, the composition comprising at least a first therapeutic agent is administered more than once. In some embodiments, the composition comprising at least a first therapeutic agent comprising a monocyte or macrophage specific engager is administered repeatedly, multiple times over a span of the therapy.

In some embodiments, the composition comprising at least a first therapeutic agent comprising a monocyte or macrophage specific engager is administered twice, thrice, four times, five times, six times, seven times, eight times, nine times, or ten times or more to a subject over a span of time comprising a few months, a year or more.

In some embodiments, the composition comprising at least a first therapeutic agent comprising a monocyte or macrophage specific engager is administered once weekly. In some embodiments, the composition comprising at least a first therapeutic agent comprising a monocyte or macrophage specific engager is administered twice weekly.

In some embodiments, the composition comprising at least a first therapeutic agent comprising a monocyte or macrophage specific engager is administered once every two weeks.

In some embodiments, the composition comprising at least a first therapeutic agent comprising a monocyte or macrophage specific engager is administered once every three weeks.

In some embodiments, the composition comprising at least a first therapeutic agent comprising a monocyte or macrophage specific engager is administered once monthly.

In some embodiments, the composition comprising at least a first therapeutic agent comprising a monocyte or macrophage specific engager is administered once in every 2 months, once in every 3 months, once in every 4 months, once in every 5 months or once in every 6 months.

In some embodiments, the composition comprising at least a first therapeutic agent comprising a monocyte or macrophage specific engager is administered by injection.

In some embodiments, the composition comprising at least a first therapeutic agent comprising a monocyte or macrophage specific engager is administered by infusion.

In some embodiments, the composition comprising at least a first therapeutic agent comprising a monocyte or macrophage specific engager is administered by intravenous infusion.

In some embodiments, the composition comprising at least a first therapeutic agent comprising a monocyte or macrophage specific engager is administered by subcutaneous infusion.

In some embodiments, treatment with monocyte or macrophage specific engagers increase the phagocytic ability and monocyte or macrophage mediated target cell killing, compared to a case where no monocyte or macrophage specific engagers were used. Monocytes or macrophages retrieved from the tumor site after treatment with monocyte or macrophage specific engagers may demonstrate a greater than 10%, or greater than 20%, or greater than 30%, or greater than 40%, or greater than 50%, or greater than 60%, or greater than 70%, or greater than 80%, or greater than 90%, or greater than 100%, or greater than 150%, or greater than 200%, or greater than 250%, or greater than 300%, or greater than 350%, or greater than 400%, or greater than 450%, or greater than 500%, or greater than 600%, or greater than 700%, or greater than 800%, or greater than 900%, or greater than 1000% increase in phagocytosis.

In some embodiments, treatment with monocyte or macrophage specific engagers increases ROS production in associated monocytes or macrophages that may be retrieved from the tumor site, compared to a case with no monocyte or macrophage specific engager treatment. Monocytes or macrophages retrieved from the tumor site after treatment with monocyte or macrophage specific engagers may demonstrate a greater than 2-fold, or greater than 3-fold, or greater than 4-fold, or greater than 5-fold, or greater than 6-fold, or greater than 7-fold, or greater than 8-fold, or greater than 9-fold, or greater than 10-fold, or greater than 20-fold, or greater than 30-fold, or greater than 40-fold, or greater than 50-fold, or greater than 60-fold, or greater than 70-fold, or greater than 80-fold, or greater than 90-fold, or greater than 100-fold, or greater than 200-fold, or greater than 300-fold, or greater than 400-fold, or greater than 500-fold, or greater than 700-fold, or greater than 800-fold, or greater than 900-fold, or greater than 1000-fold increase in ROS compared to a case with no monocyte or macrophage specific engager treatment. In some embodiments, treatment with monocyte or macrophage specific engagers increases iNOS production in associated monocytes or macrophages that may be retrieved from the tumor site, compared to a case with no monocyte or macrophage specific engager treatment. In some embodiments, treatment with monocyte or macrophage specific engagers increases respiratory burst in associated monocytes or macrophages that may be retrieved from the tumor site, compared to a case with no monocyte or macrophage specific engager treatment.

In some embodiments, treatment with monocyte or macrophage specific engagers may increase the expression of CD80 in the associated monocytes or macrophages. In some embodiments, treatment with monocyte or macrophage specific engagers may increase the expression of CD86 in the associated monocytes or macrophages compared to a no treatment. In some embodiments, treatment with monocyte or macrophage specific engagers may increase the expression of TRAIL/TNF Family death receptors in associated monocytes or macrophages compared to a case with no monocyte or macrophage specific engager treatment. In some embodiments, treatment with monocyte or macrophage specific engagers may increase the expression of LIGHT in associated monocytes or macrophages compared to a case with no monocyte or macrophage specific engager treatment. In some embodiments, treatment with monocyte or macrophage specific engagers may increase the expression of HVEM in associated monocytes or macrophages compared to a case with no monocyte or macrophage specific engager treatment. In some embodiments, treatment with monocyte or macrophage specific engagers may increase the expression of CD40 in associated monocytes or macrophages compared to a case with no monocyte or macrophage specific engager treatment. In some embodiments, treatment with monocyte or macrophage specific engagers may increase the expression of TL1A in associated monocytes or macrophages compared to a case with no monocyte or macrophage specific engager treatment. In some embodiments, treatment with monocyte or macrophage specific engagers may increase the expression of OX40L in associated monocytes or macrophages compared to a case with no monocytes or macrophages specific engager treatment. In some embodiments, treatment with monocyte or macrophage specific engagers may increase the expression of GITR in associated monocytes or macrophages compared to a case with no monocytes or macrophages specific engager treatment. In some embodiments, treatment with monocyte or macrophage specific engagers may increase the expression of SLAM in associated monocytes or macrophages compared to a case with no monocyte or macrophage specific engager treatment. In some embodiments, treatment with monocyte or macrophage specific engagers may increase the expression of CD58 in associated monocytes or macrophages compared to a case with no monocyte or macrophage specific engager treatment. In some embodiments, treatment with monocyte or macrophage specific engagers may increase the expression of CD155 in associated monocytes or macrophages compared to a case with no monocyte or macrophage specific engager treatment. In some embodiments, treatment with monocyte or macrophage specific engagers may increase the expression of CD112 in associated monocytes or macrophages compared to a case with no monocyte or macrophage specific engager treatment. In some embodiments, treatment with monocyte or macrophage specific engagers may increase the expression of B7-DC in a tumor associated myeloid cell compared to a case with no monocyte or macrophage specific engager treatment.

### EXAMPLES

### Example 1. Materials and Methods

Dulbecco modified Eagle medium, trypsin-EDTA, wortmannin (W), LY294002 (LY), Bradford reagent, and lysostaphin are purchased from Sigma-Aldrich, Inc. (St. Louis, Mo.). Reduced serum Opti-MEM I medium are purchased from Gibco-BRL (Gaithersburg, Md.). SH-5 was acquired from Enzo Life Sciences (Plymouth, Pa.), and OSU-03012 (OSU) was purchased from Cedarlane Labs (Burlington, N.C.). FuGENE transfection reagent and the 50×EDTA-free protease inhibitor cocktail are purchased from Roche Applied Science (Manheim, Germany). Cells are grown in 24-well plates to 60 to 70% confluence, and the culture medium was changed to DMEM 10% FCS. Then, in order to have a similar protein expression 5 ng of pCMV5-Akt-CA or 200 ng of pCMV5-Akt-DN in 1.2 μl of FuGENE transfection reagent (ratio, 4:1 [FuGENE-plasmid]) are added to BEC in reduced serum Opti-MEM I medium according to the manufacturer's instructions.

Cloning and characterization of the BiME, TriME and multi-specific engagers is performed in a bacterial expression system, such as in an E. coli system for test and screening purposes. Briefly, following screening of the binding domains to incorporate in an engager design, polynucleotide sequences encoding specific variable light chain and/or variable heavy chain domains are individually amplified by PCR from respective antibody-expressing clones. In case the binding domains comprise entire Fab regions, the respective regions are amplified by PCR from the respective antibody-expressing clones. The linkers are either enzymatically ligated typically to the C-terminal end of the encoded Fab or the variable domain. Alternatively, polynucleotides encoding the binding domains and the linker sequences are incorporated into plasmid by sequential cloning. In yet another alternative method, the specific sequences encoding the binding domains (Fab or variable regions) are ligated to each other by overlapping PCR, and larger inserts comprising Fab-linker-Fab designs are cloned into the expression vector to express chimeric proteins comprising the engagers.

Expressed proteins are purified and concentrated by commonly known techniques and the products are tested in experimental animals for tumor targeting and toxicity.

In some examples, a lentiviral construct comprising the chimeric proteins are used to transduce the chimeric construct in a monocyte or macrophage.

### Example 2. Construction of a Bispecific Engager (BiME) Platform

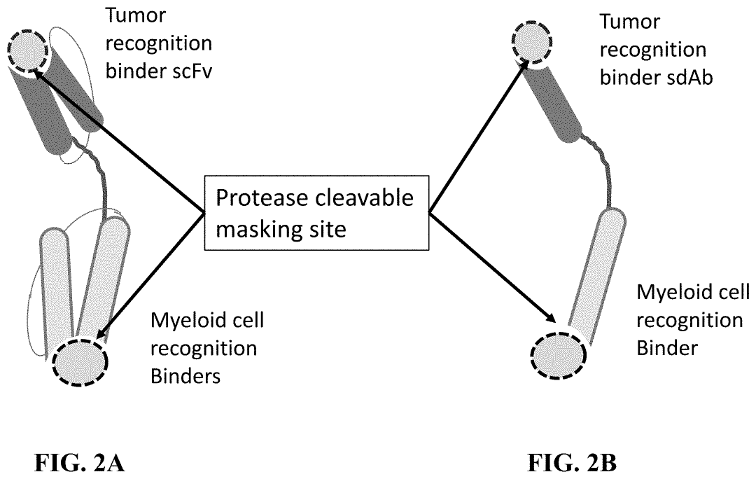

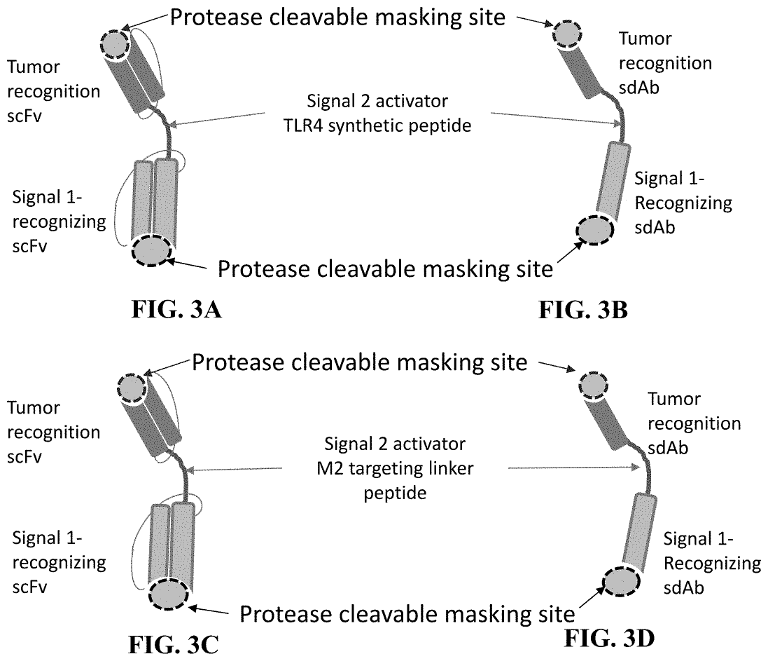

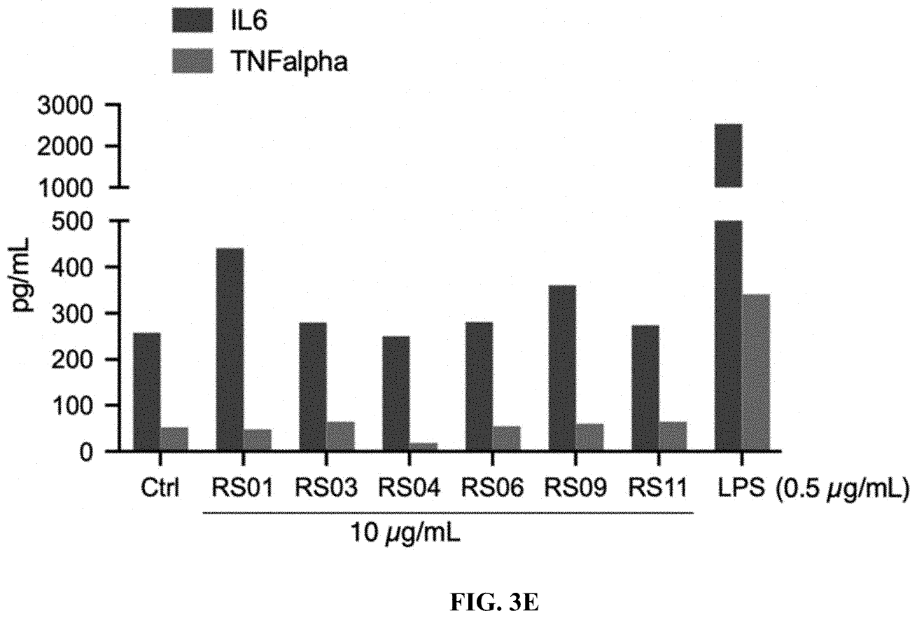

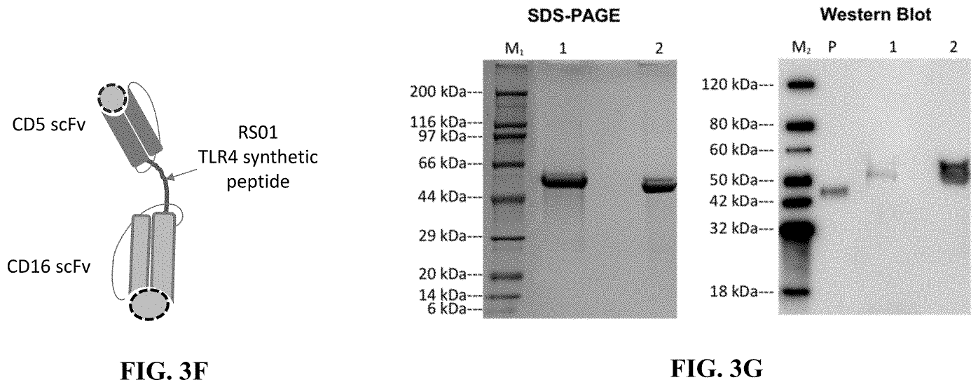

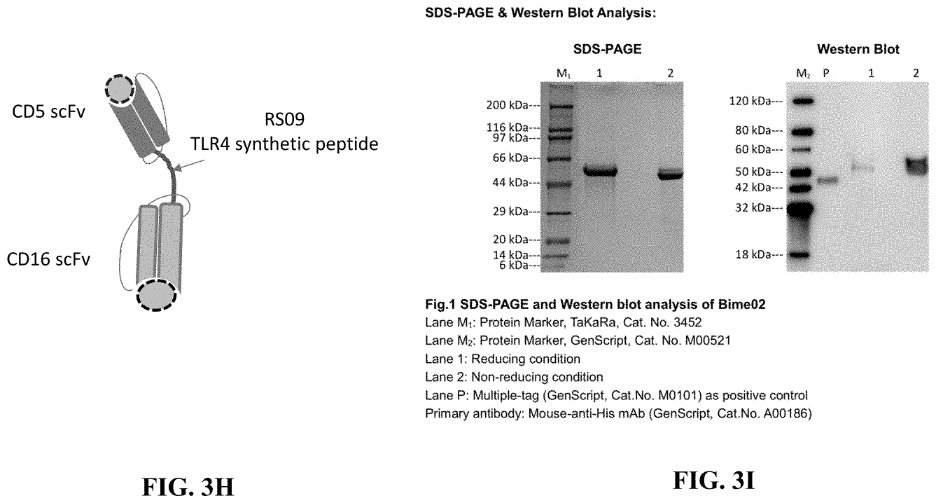

In this example, a monocyte or macrophage and tumor targets with protease masking site is designed. The bispecific engager comprises a monocyte or macrophage binding domain, which is a scavenger receptor (SRA1) binding domain. The target cell binding domain is a tumor recognition domain (e.g., TROP2). An scFv construct polypeptide design is designated in FIG. 2A. A V_{HH} construct polypeptide design is designated in FIG. 2B. In another exemplary design, the antigen binding domains are occupied with protease cleavable masking elements. Additionally, the two binding domains are linked by a TLR4 synthetic peptide. An scFv construct polypeptide design is designated in FIG. 3A. A V_{HH} construct polypeptide design is designated in FIG. 3B. The synthetic peptide is bound to two scFvs (FIG. 3A) or across two single domains (FIG. 3B). The specific TLR4 synthetic peptide linker activates TLR4 receptor on monocytes or macrophages and provides the second activation signal—Signal 2 that potentiates a monocyte or macrophage phagocytic and pro-inflammatory activity. In another exemplary design, the two binding domains of a monocyte or macrophage specific engager comprises a M2 targeting peptide. The M2 targeting peptide has an amino acid sequence of YEQDPWGVKWWY (SEQ ID NO: 116) (M2-pep) or HLSWLPDVVYAW (HLS pep) (SEQ ID NO: 117), which specifically target and bind to an M2 monocyte or macrophage which is the predominant phenotype of tumor associated monocytes or macrophages. Thus, in addition to the binding and activation by the specific binding domain, in this design a bi-specific engager can further be employed to target the engager to the specific cell, in this case an M2 monocyte or macrophage cell (FIG. 3C and FIG. 3D).

### Example 3. Construction and Expression of Bispecific Engagers

This example describes construction, expression and testing of BiMEs having activator peptide sequences within the linkers. First, a peptides sequences that were derived from different TLR activators were tested for immune activation on monocytes in culture. Exemplary TLR peptide sequences that were tested are listed below:

Table 3. TLR activator peptide sequences used as part of a linker sequences to generate bispecific engager constructs exemplified in FIG. 3A and FIG. 3B are shown in Table 3.

TABLE 3

| Sequence | |
|---|---|
| Name | Amino Acid Sequence |
| RS01 | GGQEINSSYGG (SEQ ID NO: 105) or QEINSSY (SEQ ID NO: 129) |
| RS02 | GGSHPRLSAGG (SEQ ID NO: 123) or SHPRLSA (SEQ ID NO: 130) |
| RS03 | GGSMPNPMVGG (SEQ ID NO: 106) or SMPNPMV (SEQ ID NO: 131) |
| RS04 | GGGLQQVLLGG (SEQ ID NO: 107) or GLQQVLL (SEQ ID NO: 132) |
| RS05 | GGHELSVLLGG (SEQ ID NO: 124) or HELSVLL (SEQ ID NO: 133) |
| RS06 | GGYAPQRLPGG (SEQ ID NO: 108) or YAPQRLP (SEQ ID NO: 134) |
| RS07 | GGTPRTLPTGG (SEQ ID NO: 125) or TPRTLPT (SEQ ID NO: 135) |
| RS08 | GGAPVHSSIGG (SEQ ID NO: 126) or APVHSSI (SEQ ID NO: 136) |
| RS09 | GGAPPHALSGG (SEQ ID NO: 109) or APPHALS (SEQ ID NO: 137) |
| RS10 | GGTFSNRFIGG (SEQ ID NO: 127) or TFSNRFI (SEQ ID NO: 138) |
| RS11 | GGVVPTPPYGG (SEQ ID NO: 110) or VVPTPPY (SEQ ID NO: 139) |
| RS12 | GGELAPDSPGG (SEQ ID NO: 128) or ELAPDSP (SEQ ID NO: 140) |

For testing immune response of each of the peptides, 2×10\hat{ }6 monocytes were incubated overnight with 1 microgram/ml of a peptide from Table 3, and IL6 and TNF-alpha release was measured using fluorimetric detection using Luminex 200. As shown in FIG. 3E, RS01 and RSO9 peptides induced higher IL6 release. These two peptide sequences were next selected from the pool above and utilized to generate bispecific engagers. Similarly, several more are being tested.

The bispecific or trispecific engagers can be constructed by molecular cloning. Upon generation of successful clones, each clone can be sequenced and the sequence validated. In some embodiments, a bispecific or trispecific engager comprises (i) an anti-CD5 scFv capable of binding to a CD5+ tumor cell, and (ii) an anti-CD16 scFv capable of binding to a CD16 surface molecule on a monocyte, or macrophage cell.

An exemplary anti-CD5 binder comprises a heavy chain comprising the sequence:

(SEQ ID NO: 111)
MWLQSLLLLGTVACSISEIQLVQSGGGLVKPGGSVRISCAASGYTFTNY
GMNWVRQAPGKGLEWMGWINTHTGEPTYADSFKGRFTFSLDDSKNTAYL
QINSLRAEDTAVYFCTRRGYDWYFDVWGQGTTVTV
or
(SEQ ID NO: 144)
MWLQSLLLLGTVACSISEIQLVQSGGGLVKPGGSVRISCAASGYTFTNY
GMNWVRQAPGKGLEWMGWINTHTGEPTYADSFKGRFTFSLDDSKNTAYL
QINSLRAEDTAVYFCTRRGYDWYFDVWGQGTTVTVSS.
Another exemplary anti-CD5 binder comprises a
heavy chain comprising the sequence:
(SEQ ID NO: 112)
EIQLVQSGGGLVKPGGSVRISCAASGYTFTNYGMNWVRQAPGKGLEWMG
WINTHTGEPTYADSFKGRFTFSLDDSKNTAYLQINSLRAEDTAVYFCTR
RGYDWYFDVWGQGTTVTV
or
(SEQ ID NO: 143)
EIQLVQSGGGLVKPGGSVRISCAASGYTFTNYGMNWVRQAPGKGLEWMG
WINTHTGEPTYADSFKGRFTFSLDDSKNTAYLQINSLRAEDTAVYFCTR
RGYDWYFDVWGQGTTVTVSS.

An exemplary anti-CD5 binder may comprise a light chain comprising the amino acid sequence

(SEQ ID NO: 113)
DIQMTQSPSSLSASVGDRVTITCRASQDINSYLSWFQQKPGKAPKTLIY
RANRLESGVPSRFSGSGSGTDYTLTISSLQYEDFGIYYCQQYDESPWTF
GGGTKLEIK

An exemplary a bispecific or trispecific engager, such as bispecific or trispecific engager containing an anti-CD5 scFv may comprise a short peptide linker connecting an exemplary heavy chain and an exemplary light chain, having a sequence: SSGGGGSGGGGSGGGGS (SEQ ID NO: 114) or SGGGGS (SEQ ID NO: 145) or GGGGS (SEQ ID NO: 146) or GGGG (SEQ ID NO: 147).

An exemplary anti-CD5 scFv comprises an amino acid sequence:

(SEQ ID NO: 115)
MWLQSLLLLGTVACSISEIQLVQSGGGLVKPGGSVRISCAASGYTFTNY
GMNWVRQAPGKGLEWMGWINTHTGEPTYADSFKGRFTFSLDDSKNTAYL
QINSLRAEDTAVYFCTRRGYDWYFDVWGQGTTVTVSSGGGGSGGGGSGG
GGSDIQMTQSPSSLSASVGDRVTITCRASQDINSYLSWFQQKPGKAPKT
LIYRANRLESGVPSRFSGSGSGTDYTLTISSLQYEDFGIYYCQQYDESP
WTFGGGTKLEIK

An exemplary anti-CD16 scFv can comprise a heavy chain variable sequence comprising the amino acid sequence:

(SEQ ID NO: 141)
QVQLVQSGAEVKKPGESLKVSCKASGYTFTSYYMHWVRQAPGQGLEWMG
IINPSGGSTSYAQKFQGRVTMTRDTSTSTVYMELSSLRSEDTAVYYCAR
GSAYYYDFADYWGQGTLVTVSS

An exemplary anti-CD16 scFv can comprise a light chain variable sequence comprising the amino acid sequence:

(SEQ ID NO: 142)
SYVLTQPSSVSVAPGQTATISCGGHNIGSKNVHWYQQRPGQSPVLVIYQ
DNKRPSGIPERFSGSNSGNTATLTISGTQAMDEADYYCQVWDNYSVLFG
GGTKLTVL

The two scFvs can be linked by a synthetic peptide linker that comprises one of the TLR activating peptide sequences, such as those described in Table 3. The constructs are thereafter named as Binder 1-linker-Binder 2, such as, CD5-RS01-CD16, having an RS01 TLR activating peptide sequence in the linker; or, CD5-RS09-CD16, having an RS01 TLR activating peptide sequence in the linker, as shown in FIG. 3F, or FIG. 3H respectively. Sequence verified clones are then expressed in a suitable cell and the protein is detected by gel migration using molecular markers and western blot, using a suitable positive control.

An exemplary bispecific or trispecific engager can comprise the sequence: METDTLLLWVLLLWVPGSTG (SEQ ID NO: 148) or any other useful leader sequence.

An exemplary bispecific or trispecific engager can comprise the sequence: HHHHHH (SEQ ID NO: 149) or any other useful affinity tag.

An exemplary bispecific or trispecific engager can comprise the sequence: ENLYFQG (SEQ ID NO: 150) or any other useful protease cleavage sequence.

An exemplary bispecific or trispecific engager can comprise a first scFv comprising a variable heavy chain linked to a variable light chain via a first linker, which can be linked to a second scFv via a second linker, wherein the second scFv comprises a variable heavy chain linked to a variable light chain via a third linker. In some embodiments the second linker comprises a TLR activating peptide sequence, such as those described in Table 3. In some embodiments, the first linker has a length of 1, 2, 3, 4, 5, 6, 7, 8, 9, 10, 11, 12, 13, 14, 15, 16, 17, 18, 19, or 20 or more amino acids. In some embodiments, the second linker has a length of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more amino acids. In some embodiments, the third linker has a length of 1, 2, 3, 4, 5, 6, 7, 8, 9, 10, 11, 12, 13, 14, 15, 16, 17, 18, 19, or 20 or more amino acids.

(SEQ ID NO: 151)
METDTLLLWVLLLWVPGSTGHHHHHHENLYFQGEIQLVQSGGGLVKPGG
SVRISCAASGYTFTNYGMNWVRQAPGKGLEWMGWINTHTGEPTYADSFK
GRFTFSLDDSKNTAYLQINSLRAEDTAVYFCTRRGYDWYFDVWGQGTTV
TVSSsggggsSYVLTQPSSVSVAPGQTATISCGGHNIGSKNVHWYQQRP
GQSPVLVIYQDNKRPSGIPERFSGSNSGNTATLTISGTQAMDEADYYCQ
VWDNYSVLFGGGTKLTVLggggQEINSSYggggsQVQLVQSGAEVKKPG
ESLKVSCKASGYTFTSYYMHWVRQAPGQGLEWMGIINPSGGSTSYAQKF
QGRVTMTRDTSTSTVYMELSSLRSEDTAVYYCARGSAYYYDFADYWGQG
TLVTVSSsggggsDIQMTQSPSSLSASVGDRVTITCRASQDINSYLSWF
QQKPGKAPKTLIYRANRLESGVPSRFSGSGSGTDYTLTISSLQYEDFGI
YYCQQYDESPWTFGGGTKLEIK

An exemplary bispecific or trispecific engager can comprise the sequence:

(SEQ ID NO: 152)
METDTLLLWVLLLWVPGSTGHHHHHHENLYFQGEIQLVQSGGGLVKPGG
SVRISCAASGYTFTNYGMNWVRQAPGKGLEWMGWINTHTGEPTYADSFK
GRFTFSLDDSKNTAYLQINSLRAEDTAVYFCTRRGYDWYFDVWGQGTTV
TVSSsggggsSYVLTQPSSVSVAPGQTATISCGGHNIGSKNVHWYQQRP
GQSPVLVIYQDNKRPSGIPERFSGSNSGNTATLTISGTQAMDEADYYCQ
VWDNYSVLFGGGTKLTVLggggAPPHALSggggsQVQLVQSGAEVKKPG
ESLKVSCKASGYTFTSYYMHWVRQAPGQGLEWMGIINPSGGSTSYAQKF
QGRVTMTRDTSTSTVYMELSSLRSEDTAVYYCARGSAYYYDFADYWGQG
TLVTVSSsggggsDIQMTQSPSSLSASVGDRVTITCRASQDINSYLSWF
QQKPGKAPKTLIYRANRLESGVPSRFSGSGSGTDYTLTISSLQYEDFGI
YYCQQYDESPWTFGGGTKLEIK

Expression of the CD5-RS01-CD16 BiME is shown by SDS PAGE FIG. 3G (left) and by western blot in FIG. 3G (right) under reducing and non-reding gel electrophoresis as indicated. Expression of the CD5-RSO9-CD16 BiME is shown by SDS PAGE FIG. 3I (left) and by western blot in FIG. 3I (right) under reducing and non-reding gel electrophoresis as indicated. These data demonstrate successful generation and expression of the TLR-activating sequence containing bispecific engagers. The engagers described above are tested in in vitro. A microparticle based phagocytosis assay was used to examine changes in phagocytosis. Briefly, streptavidin coupled fluorescent polystyrene microparticles (6 μm diameter) are conjugated with biotinylated recombinantly expressed and purified cancer ligand, in this case CD5. Myeloid cells cultured in presence of the beads and an engager protein, are incubated with the ligand coated microparticles for 1-4 h and the amount of phagocytosis was analyzed and quantified using flow cytometry.

### Example 4. Trispecific Engager (TriME) Design

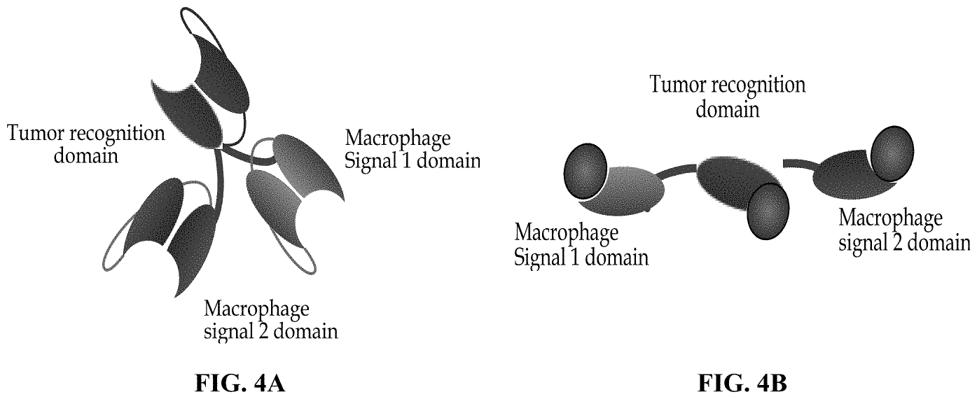

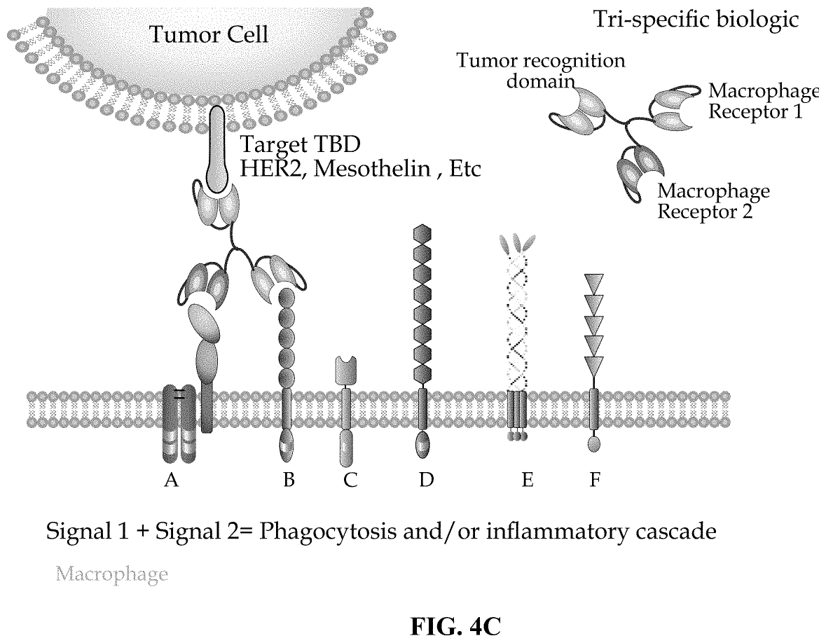

In this example a design of a trispecific antigen-binding protein is set forth as follows. The trispecific antigen-binding protein comprises (a) a first domain (A) which specifically binds to human Scavenger/Phagocytic receptor; (b) a second domain (B) which is a danger signal receptor; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2 (FIG. 4A). The antigen binding domains are occupied with protease cleavable masking elements, which are activated by availability and contact with the protease. An exemplary nanobody design is shown in FIG. 4B. FIG. 4C provides a graphical view of a trispecific engager and an exemplary nature of function on a target tumor cell and a monocyte or macrophage. The trispecific engager structurally comprises a tumor recognition or tumor binding domain, a monocyte or macrophage receptor 1 binding domain, and a monocyte or macrophage receptor 2 binding domain (shown in inset on the top right corner of FIG. 4C). As an expected functional mode, the tumor binding domain binds to the target surface molecule (tumor antigen) of a tumor cell, while the two monocyte or macrophage receptors, B and A are bound respectively by the monocyte or macrophage receptor 1 binding domain, and the monocyte or macrophage receptor 2 binding domain of the trispecific engager. As shown in the figure, engagement of a receptor A and B, by the trispecific engager also operably linked with the tumor antigen by the tumor recognition domain, provides a Signal 1 and a Signal 2 to the monocyte or macrophage. The dual signal (Signal 1+Signal 2) activates the monocyte or macrophage thereby enhancing phagocytosis and activating an inflammatory cascade in this exemplary figure, which lead to phagocytic killing of the target cell.

### Example 5. Antigen Binding Domain Masking Design

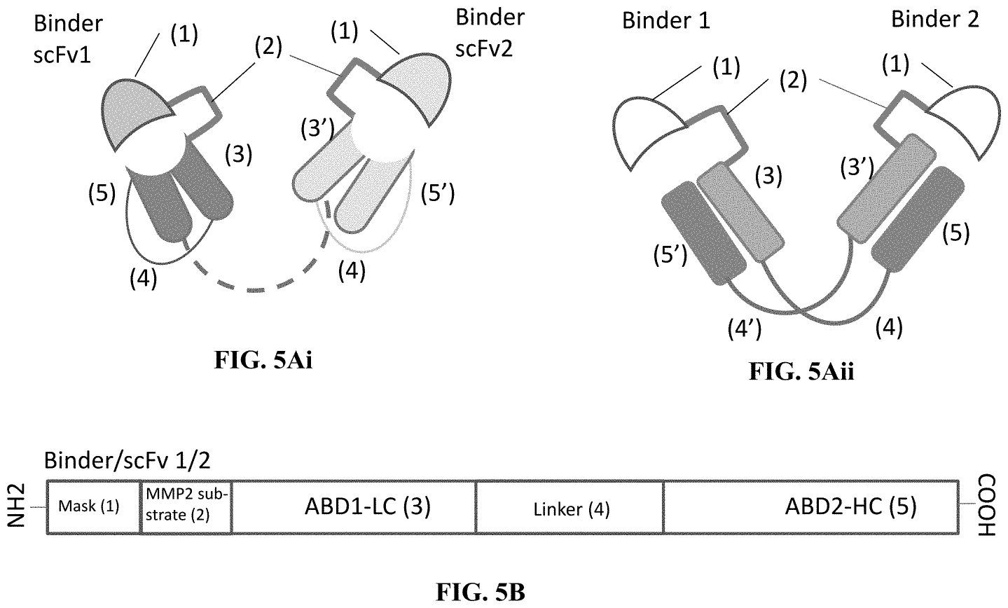

In this example, a generalized exemplary design for an engager having masked antigen binding domains is described in further detail. FIG. 5Ai is a diagrammatic representation of a bispecific engager with two scFV binders, scFv1, and scFv2. SdAb or diabody engagers can also be likewise constructed with necessary structural modifications, an exemplary diabody construct with two binders is represented in FIG. 5Aii. The antigen binding domains are masked by a peptide mask (1) that remains bound to the antigen binding portions of the diabody ABD1 and ABD2, linked at the N terminal portion of the light chain variable domain of ABD1 (3) of the first chain, or the light chain variable region of ABD2 of the second chain by a peptide linker (2). The peptide linker joining the mask with the light chain variable domains is a substrate for matrix metalloproteinase 2 (MMP2) substrate, having an amino acid sequence GPLGVR. The design allows passing of the masked diabody engager to pass through the circulation without binding to any substrate until MMP2 is available to cleave the linking peptide. It is understood that the cancer microenvironment is rich in MMP2. Therefore, the diabody engager is activated in a cancer microenvironment to bind its target cancer cell and the monocyte or macrophage with ABD1 and ABD2 respectively in a tumor environment. FIG. 5B exemplifies the nucleic acid construct of a single chain of a diabody. The nucleic acid construct comprises from 5′-3′ end a nucleic acid sequence encoding the mask peptide, a MMP2 linker, a sequence encoding ABD1 light chain (ABD1-LC), which is linked to a nucleic acid sequence encoding a peptide linker that joins with the ABD1-LC and ABD2-HC; followed by the nucleic acid sequence encoding the ABD2 HC.

### Example 6. Modular Antigen Binding Engager Designs

In this example, several modular designs of binding domains represented by light chain heavy chain domains arranged on an antibody-like polypeptide structure as shown in FIG. 6. In one design, a common light chain is used to pair with two non-identical heavy chains in an IgG like structure, thereby rendering a bispecific binding domain that could be used in a bispecific or a trispecific engager design. In another model depicted herein, a chimeric bi- or trispecific engager uses a combination of an scFv joined to one arm of an usual antibody light and heavy chain combination. In one design, two scFvs replace the heavy chain-light chain paired regions, while the scFvs are connected by the constant regions of the heavy chains. In other designs as depicted herein, one or more scFvs may be conjugated to the Fc region. In yet other designs, one or more scFvs may be conjugated to the constant regions as side chains of an IgG like polypeptide.

### Example 7. Use of Monocyte or Macrophage Specific Activators in an Engager for Activating Inflammatory Signal in Monocyte or Macrophage an Potentiating Phagocytosis

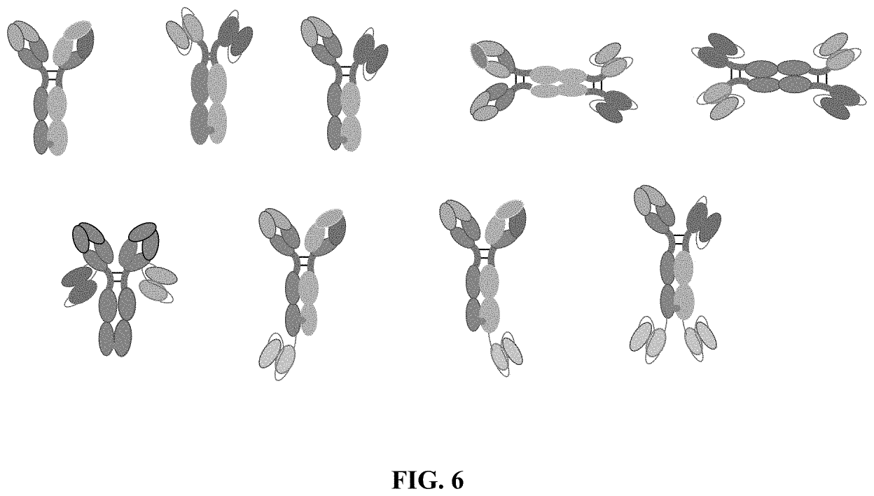

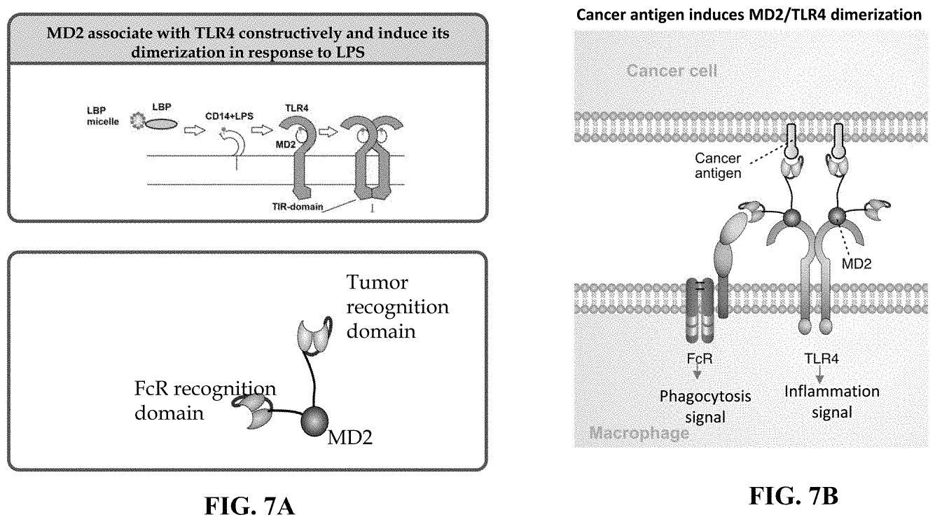

MD2 can bind to and activate TLR4 in response to LPS, as shown in the diagrammatic representation in FIG. 7A, upper panel. In an exemplary design, MD2 is constructed into a monocyte or macrophage specific engager, where the MD2, in addition to the tumor specific binding domain and the monocyte or macrophage specific binding domain associates with TLR4 receptors, and help in the dimerization of TLR4 receptors on the monocyte or macrophage thereby sending a monocyte or macrophage activating signal (Signal 2) that further potentiates the inflammatory activation and phagocytic killing of a target cancer cell by the monocyte or macrophage (FIG. 7B).

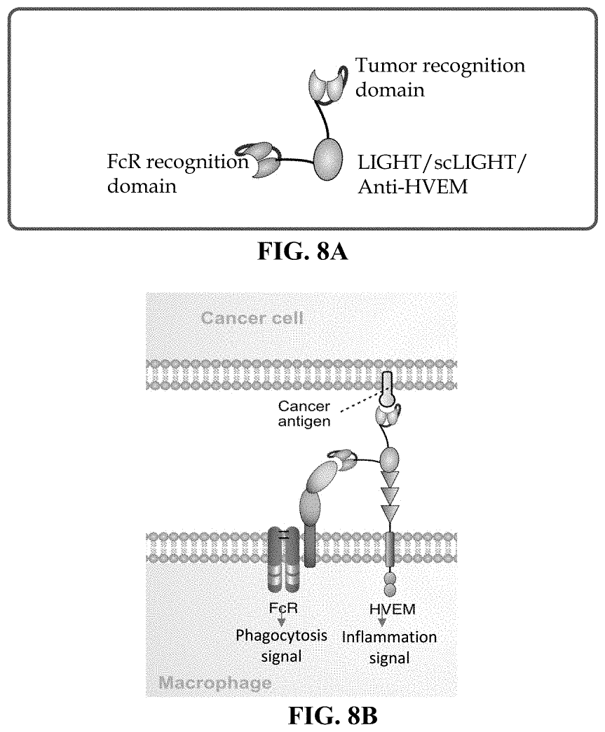

In another example, Herpes Virus Entry Mediator (HVEM) and its association with tumor necrosis factor (TNF)-related 2 (LIGHT) is exploited in designing an exemplary monocyte or macrophage specific engager that potentiates monocyte or macrophage effector functions. HVEM is a member of the TNFR superfamily and has two more ligands: HSV surface envelope gD and LT□. It is expressed on T cells, B cells, NK cells, monocytes, neutrophils, and DC. The LIGHT-HVEM interaction increased levels of phagocytosis, interleukin (IL)-8, TNF-□, nitric oxide (NO), and reactive oxygen species (ROS) in monocytes and neutrophils. In an exemplary design, a monocyte or macrophage specific engager comprises a LIGHT domain that can bind to HVEM. In a variation of the design, the LIGHT domain that binds to HVEM may be replaced by an agonist antibody of antigen binding domain that binds to HVEM, as shown in FIG. 8A. The corresponding mode of function of the engager is depicted graphically in FIG. 8B, where binding of the LIGHT domain with monocyte or macrophage associated HVEM activates an inflammatory signal (Signal 2) in the monocyte or macrophage, that potentiates its effector functions as a phagocytic cell.

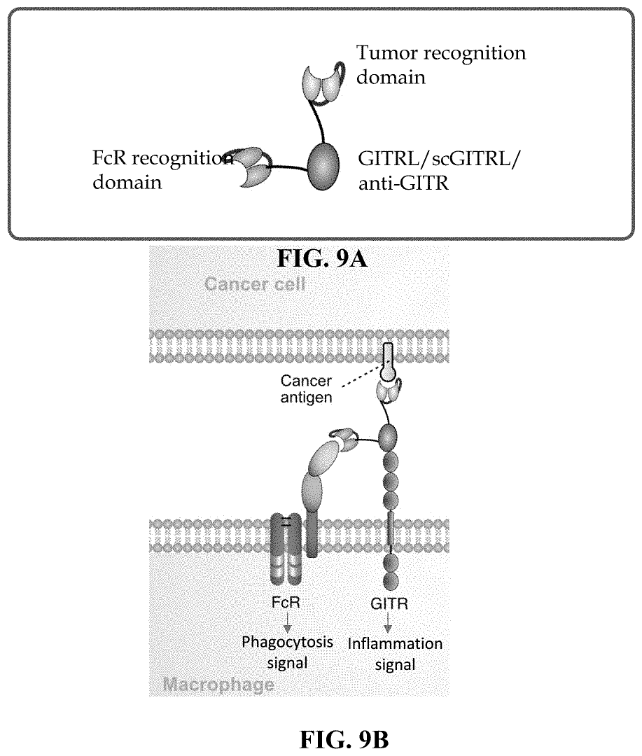

In another example, GIRT associated activation signal is exploited in an exemplary monocyte or macrophage specific engager design that is shown in FIG. 9A. GIRT is expressed on monocytes or macrophages, and when bound by its ligand, GIRTL, it generates an inflammatory signal in the monocyte or macrophage, as depicted graphically in FIG. 9B.

### Example 8. Use of Linkers with Sequences to Facilitate Accelerated Association with in an Engager

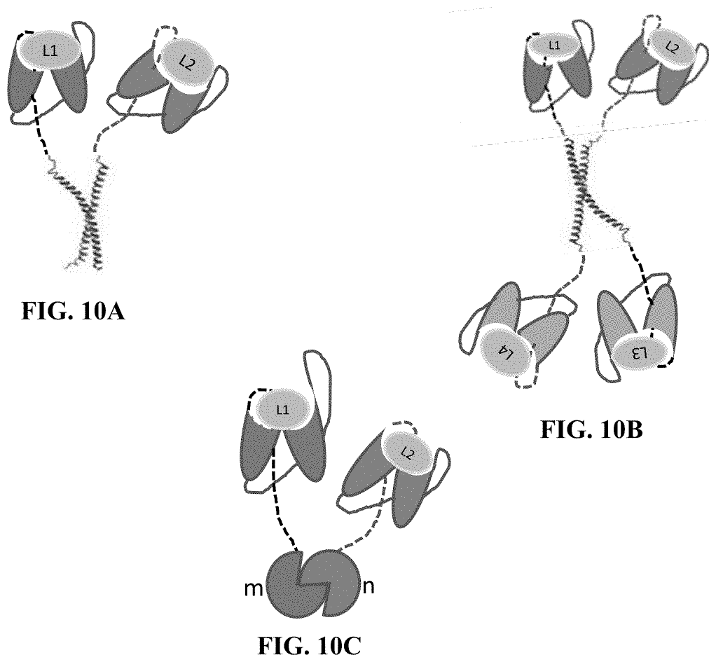

In this example, monocyte or macrophage specific engagers are designed to have linkers between multi-specific binding domains that have complementarity to each other. FIG. 10A-FIG. 10C demonstrates exemplary designs which include leucine zipper domains, (FIGS. 10A and 10B) or rationally designed synthetic sequences comprising a complementary binding region (FIG. 10C). Exemplary linker sequences disclosed in the specification are used. In specific constructs linker domains are utilized that dimerize of trimerize, bringing useful domains in closer proximity. Shown in these figures are exemplary use of leucine zipper domains and coupling protein domains in binding heteromeric binder domains closer together.

### Example 9. Screen for Selecting a Myeloid Cell Binder Domains

In this example, a screen is undertaken to select the cell surface molecules on a myeloid cell, or functional fragments thereof, that can be useful to design binding domains for engagers described herein. A binding domain can be a phagocytosis receptor engager or activator. As is now understood, not all phagocytic cell surface receptors on a phagocytic cell have equal ability to be induced or activated to generate proinflammatory signaling or in any way potentiate monocyte or macrophage effector functions. Hence, to harness monocytes or macrophages and other myeloid cells to kill cancer, a series of signal 1 and signal 2 targets are generated on myeloid cells. These targets were identified through the screening of materials associated with inflammation as well as immune tolerance.

This is done using a unique tool that uses proprietary arrays of expression vectors—encoding over 5,500 full-length human plasma membrane and tethered secreted proteins—spotted onto slides. Human cells are grown over the top become reverse-transfected resulting in cell surface expression of each respective protein at distinct slide locations. The test formulation is then applied and specific binding analyzed and confirmed using an appropriate detection system. These hits were then interrogated and examined as potential targets for monocyte or macrophage binding and modulation.

Specific useful binding agents, or domains identified from the screens are then reverse transcribed, and cloned into lentiviral expression vectors to generate the second binding domain or an engager BiME or TriME constructs. A recombinant nucleic acid encoding a BiMEs or TriMEs can generated using one or more domains from highly phagocytic receptor binding domains generated from the screen.

What is claimed is:

1. A composition comprising a first therapeutic agent, wherein the therapeutic agent comprises:
(a) a first binding domain, wherein the first binding domain is a first antibody or functional fragment thereof that specifically interacts with an antigen on a target cell, and
(b) a second binding domain, wherein the second binding domain is a second antibody or functional fragment thereof that specifically interacts with a myeloid cell;
wherein,
(i) the first therapeutic agent is coupled to a first component, wherein the first component is an additional therapeutic agent or a third binding domain, or
(ii) the composition comprises an additional therapeutic agent.

2. A composition comprising a therapeutic agent, wherein the therapeutic agent comprises: (a) a first binding domain that specifically interacts with an antigen of a target cell, (b) a second binding domain that specifically interacts with a myeloid cell, and (c) a third binding domain that specifically interacts with the myeloid cell.

3. The composition of claim 1 or 2, wherein the myeloid cell is a monocyte cell or a macrophage cell.

4. The composition of any one of claims 1-3, wherein the second binding domain that specifically interacts with a myeloid cell interacts with a phagocytic or tethering receptor of the myeloid cell.

5. The composition of claim 2, wherein the third binding domain that specifically interacts with a myeloid cell interacts with an extracellular region of a first phagocytic or tethering receptor of the myeloid cell.

6. The composition of any one of claims 1-5, wherein any one of binding domains of the therapeutic agent comprises the binding domain of a an antibody, a functional fragment of an antibody, a variable domain thereof, a V_{H} domain, a V_{L} domain, a VNAR domain, a V_{HH} domain, a single chain variable fragment (scFv), an Fab, a single-domain antibody (sdAb), a nanobody, a bispecific antibody, a diabody, or a functional fragment or a combination thereof.

7. The composition of any one of claims 1-6, wherein the antigen on the target cell to which the first binding domain binds, is a cancer antigen or a pathogenic antigen on the target cell or an autoimmune antigen.

8. The composition of any one of claims 1-7, wherein the antigen on the target cell to which the first binding domain binds, is a viral antigen.

9. The composition of any one of claims 1-8, wherein the antigen on the target cell to which the first binding domain binds is a T-lymphocyte antigen.

10. The composition of any one of claims 1-9, wherein the antigen on the target cell to which the first binding domain binds is an extracellular antigen.

11. The composition of any one of claims 1-9, wherein the antigen on the target cell to which the first binding domain binds is an intracellular antigen.

12. The composition of any one of claims 1-11, wherein the antigen on the target cell to which the first binding domain binds is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor vIII (EGFRvIII), Mesothelin, Human Epidermal Growth Factor Receptor 2 (HER2), Mesothelin, EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFR-beta, SSEA-4, EGFR, NCAM, prostase, PAP, ELF2M, GM3, TEM7R, CLDN6, TSHR, GPRC5D, ALK, IGLL1 and combinations thereof.

13. The composition of any one of claims 1-12, wherein the antigen on the target cell to which the first binding domain binds is selected from the group consisting of CD2, CD3, CD4, CD5, CD7, CCR4, CD8, CD30, CD45, and CD56.

14. The composition of any one of claims 12 or 13, wherein the antigen on the target cell to which the first binding domain binds is an ovarian cancer antigen or a T lymphoma antigen.

15. The composition of any one of preceding claims, wherein the antigen on the target cell to which the first binding domain binds is an integrin receptor.

16. The composition of claim 1 or 2, wherein the second binding domain or the third binding domain binds to an integrin receptor.

17. The composition of claim 16, wherein the second binding domain or the third binding domain binds to an integrin receptor selected from the group consisting of α1, α2, αIIb, α3, α4, α5, α6, α7, α8, α9, α10, α11, αD, αE, σL, αM, αV, αX, β1, β2, β3, β4, β5, β6, β7, and β8.

18. The composition of any one of the preceding claims, wherein the therapeutic agent binds to a phagocytic or tethering receptor that comprises a phagocytosis activation domain.

19. The composition of claim 18, wherein the therapeutic agent binds to a receptor or a protein selected from the group consisting of the receptors listed in Table 2A and Table 2B, or a fragment thereof.

20. The composition of claim 18, wherein the therapeutic agent binds to a phagocytic receptor selected from the group consisting of lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor I, CR1, CD35, CR3, CR4, Tim-1, Tim-4 and CD169.

21. The composition of any one of claims 1-20, wherein the therapeutic agent binds to a receptor comprising an intracellular signaling domain that comprises a pro-inflammatory signaling domain.

22. The composition of any one of claims 1-21, wherein the first therapeutic agent comprises a polypeptide that is less than 1000 amino acids or 1000 nm in length.

23. The composition of any one of claims 1-22, wherein the first therapeutic agent comprises a polypeptide that is less than 500 amino acids or 500 nm in length.

24. The composition of any one of claims 1-23, wherein the first therapeutic agent comprises a polypeptide that is 200-1000 amino acids or 200-1000 nm in length.

25. The composition of any one of claims 1-24, wherein engagement of the binding domains of the first therapeutic agent contacts the cancer cell to the myeloid cell.

26. The composition of claim 1, wherein the second binding domain specifically interacts with a myeloid cell and promotes phagocytosis activity of the myeloid cell.

27. The composition of claim 1, wherein the second binding domain specifically interacts with a myeloid cell and promotes inflammatory signaling of the myeloid cell.

28. The composition of claim 1, wherein the second binding domain specifically interacts with a myeloid cell or an adhesion molecule and promotes adhesion of the myeloid cell to the target cell.

29. The composition of claim 1, wherein the second binding domain specifically interacts with a myeloid cell and inhibits anti-phagocytic activity of the myeloid cell mediated by the target cell.

30. The composition of claim 1, wherein the second binding domain specifically interacts with a myeloid cell and inhibits anti-inflammatory activity of the myeloid cell mediated by the target cell.

31. The composition of claim 2, wherein the second and/or the third binding domain promotes phagocytic activity of the myeloid cell.

32. The composition of claim 2, wherein the second and/or the third binding domain promotes inflammatory signaling of the myeloid cell.

33. The composition of claim 2, wherein the second and/or the third binding domain specifically interacts with a myeloid cell or an adhesion molecule and promotes adhesion of the myeloid cell to the target cell.

34. The composition of claim 2, wherein the second and/or the third binding domain inhibits anti-phagocytic activity of the myeloid cell mediated by the target cell.

35. The composition of claim 2, wherein the second and/or the third binding domain inhibits anti-inflammatory activity of the myeloid cell mediated by the target cell.

36. The composition of any one of the preceding claims, wherein the therapeutic agent comprises a therapeutic polypeptide.

37. The composition of any one of the preceding claims, wherein the therapeutic agent comprises a recombinant nucleic acid encoding the therapeutic polypeptide.

38. The composition of claim 1, wherein the third binding domain or the additional therapeutic agent comprises a CD47 antagonist, a CD47 blocker, an antibody, a chimeric CD47 receptor, a sialidase, a cytokine, a proinflammatory gene, a procaspase, or an anti-cancer agent.

39. The composition of any one of the preceding claims, wherein the first binding domain, the second binding domain and the third binding domain bind to distinct non-identical target antigens.

40. The composition of claim 1 or 2, wherein the first binding domain, the second binding domain or the third binding domain is a ligand binding domain.

41. The composition of any one of the preceding claims, wherein the first, the second or the third binding domains are operably linked by one or more linkers.

42. The composition of claim 41, wherein the linker is a polypeptide.

43. The composition of claim 42, wherein the linker is a functional peptide.

44. The composition of claim 43, wherein the linker is a ligand for a receptor.

45. The composition of claim 44, wherein the linker is a ligand for a monocyte or macrophage receptor.

46. The composition of claim 43 or 44, wherein the linker activates the receptor.

47. The composition of claim 43 or 44, wherein the linker inhibits the receptor.

48. The composition of claim 44, wherein the linker is a ligand for a M2 macrophage receptor.

49. The composition of claim 43 or 44, wherein the linker is a ligand for a TLR receptor, such as TLR4.

50. The composition of claim any of the claims 43, 44, 45, 46, 48 or 49, wherein the linker activates a TLR receptor.

51. The composition of any one of the preceding claims, wherein the first, the second and/or the third binding domains are associated with a mask that binds to the binding domain.

52. The composition of claim 51, wherein the mask is an inhibitor that inhibits the interaction of binding domain to its target when the mask remains associated with the respective binding domain.

53. The composition of claim 52, wherein the mask is associated with the binding domain via a peptide linker.

54. The composition of claim 53, wherein the peptide linker comprises a cleavable moiety.

55. The composition of claim 53, wherein the cleavable moiety is cleaved by a protein or an enzyme selectively abundant in the site of the cancer or tumor.

56. The composition of any one of claims 1-55, wherein the third binding domain that specifically interacts with an extracellular region of a second receptor of the macrophage activates the macrophage.

57. The composition of any one of claims 1-56, wherein upon binding of the therapeutic agent to the myeloid cell, the killing or phagocytosis activity of the myeloid cell is increased by at least 10%, or 20%, or 30%, or 40%, or 50%, or 60%, or 70% or 90% or 100% compared to a myeloid cell not bound by the therapeutic agent, as measured by a particle uptake assay.

58. The composition of any one of claims 1-57, wherein engagement of the binding domains of first therapeutic agent triggers phagocytosis of the cancer cell by the myeloid cell.

59. The composition of any one of claims 1-58, wherein engagement of the additional therapeutic agent potentiates or increases the phagocytic killing of the cancer cell by the myeloid cell.

60. The composition of any one of claims 1-59, wherein the second or third binding domain binds to an extracellular of IgA, IgD, IgE, IgG, IgM, FcγRI, FcγRIIA, FcγRIIB, FcγRIIC, FcγRIIIA, FcγRIIIB, FcRn, TRIM21, FcRL5.

61. The composition of any one of claims 1-60, wherein the second or the third binding domain comprises an M2 domain.

62. The composition of any one of claims 1-61, wherein the second or the third binding domain comprises a LIGHT domain.

63. The composition of any one of claims 1-62, wherein the second or the third binding domain comprises a HVEM domain.

64. The composition of any one of claims 1-63, wherein the second or the third binding domain comprises a GITR domain.

65. A pharmaceutical composition comprising:
a first therapeutic agent, wherein the therapeutic agent comprises one or more polypeptides or recombinant nucleic acids encoding the one or more polypeptides, wherein the one or more polypeptides comprise:
(a) a first binding domain, wherein the first binding domain is a first antibody or functional fragment thereof that specifically interacts with an antigen of a target cell, and
(b) a second binding domain, wherein the second binding domain is a second antibody or functional fragment thereof that specifically interacts with a myeloid cell;
wherein,
(i) the first therapeutic agent is coupled to a first component, wherein the first component is an additional therapeutic agent or a third binding domain, or
(ii) the composition comprises an additional therapeutic agent; and
an acceptable pharmaceutical salt or excipient.

66. The pharmaceutical composition of claim 65, wherein the first therapeutic agent comprises a single polypeptide.

67. The pharmaceutical composition of claim 65, wherein the first therapeutic agent comprises multiple polypeptides.

68. The pharmaceutical composition of claim 65, wherein the first therapeutic agent is a recombinant nucleic acid encoding the one or more polypeptides.

69. The pharmaceutical composition of claim 65, further comprising a second therapeutic agent.

70. A method of treating a disease or condition in a subject in need thereof, comprising:
administering to the subject a pharmaceutical composition, comprising:
a first therapeutic agent, wherein the therapeutic agent comprises one or more polypeptides or recombinant nucleic acids encoding the one or more polypeptides, wherein the one or more polypeptides comprise:
(a) a first binding domain, wherein the first binding domain is a first antibody or functional fragment thereof that specifically interacts with an antigen of a target cell, and
(b) a second binding domain, wherein the second binding domain is a second antibody or functional fragment thereof that specifically interacts with a myeloid cell;
wherein,
(i) the first therapeutic agent is coupled to a first component, wherein the first component is an additional therapeutic agent or a third binding domain, or
(ii) the composition comprises an additional therapeutic agent; and
an acceptable pharmaceutical salt or excipient.

71. The method of claim 70, further comprising, administering a second therapeutic agent.

72. The method of claim 70, wherein the administering the pharmaceutical composition comprises administering the pharmaceutical composition intravenously.

73. The method of claim 70, wherein the administering the pharmaceutical composition comprises administering the pharmaceutical composition subcutaneously.

74. The method of claim 70, wherein the administering the pharmaceutical composition comprises injecting the pharmaceutical composition.

75. The composition of claim 1 or 2, wherein the first binding domain comprises a sequence having an amino acid sequence with at least 80%, 85%, 90%, 95% or 100% sequence identity to a sequence selected from the group consisting of SEQ ID NOs: 27, 28, 111, 112, 113, 115, 143 and 144.

76. The composition of claim 1 or 2, wherein the second binding domain comprises a sequence having an amino acid sequence with at least 80%, 85%, 90%, 95% or 100% sequence identity to a sequence selected from the group consisting of SEQ ID NOs: 141 and 142.

77. The composition of claim 1, wherein the first component comprises an amino acid sequence GGQEINSSYGG (SEQ ID NO: 105) or QEINSSY (SEQ ID NO: 129).
(SEQ ID NO: 105)
GGQEINSSYGG
or
(SEQ ID NO: 129)
QEINSSY.

78. The composition of claim 1, wherein the first component comprises an amino acid sequence
(SEQ ID NO: 109)
GGAPPHALSGG
or
(SEQ ID NO: 137)
APPHALS.

79. The composition of claim 49 or 50, wherein the linker comprises an amino acid sequence GGQEINSSYGG (SEQ ID NO: 105), or QEINSSY (SEQ ID NO: 129) or GGAPPHALSGG (SEQ ID NO: 109) or APPHALS (SEQ ID NO: 137).
(SEQ ID NO: 105)
GGQEINSSYGG,
or
(SEQ ID NO: 129)
QEINSSY
or
(SEQ ID NO: 109)
GGAPPHALSGG
or
(SEQ ID NO: 137)
APPHALS.

80. A bispecific or trispecific engager, comprising a sequence having an amino acid sequence with at least 80%, 85%, 90%, 95% or 100% sequence identity to SEQ ID NO: 151.

81. A bispecific or trispecific engager, comprising a sequence having an amino acid sequence with at least 80%, 85%, 90%, 95% or 100% sequence identity to SEQ ID NO: 152.


SEQUENCE LISTING (160 sequences)

SEQ ID NO: 1 - protein, 126 aa, Artificial Sequence
  Description of Artificial Sequence Synthetic polypeptide
  MOD_RES (81)..(81): Any amino acid
EVQLVESGGGLVQAGGSLRLSCTASGRAVSTYAMGWFRQAPGKEREFVAAMISSLSSKSYADTVKGRFTISRDYAKNTVYXQMNSLKPEDTADYYCAADLLPYSSSRSLPMGYDYWGQGTQVTVSS

SEQ ID NO: 2 - protein, 126 aa, Artificial Sequence
  Description of Artificial Sequence Synthetic polypeptide
EVQLVESGGGLVQAGGSLRLSCTASGRAVSTYAMGWFRQAPGKEREFVAAMISSLSSKSYADSVKGRFTISRDYAKNTVYLQMNSLKPEDTADYYCAADLLPYSSTRSLPMGYDYWGQGTQVTVSS

SEQ ID NO: 3 - protein, 127 aa, Artificial Sequence
  Description of Artificial Sequence Synthetic polypeptide
EVQLVESGGGLVQAGGSLRLSCAASGSFSLYDMGWFSQAPGKEREFVAAINWSGGSTAYADSVKGRFTISRDSAKNTVYLQMNSLKPEDTAVYYCAAKPAKYHFGSGYRDFAEYPYWGQGTQVTVSS

SEQ ID NO: 4 - protein, 123 aa, Artificial Sequence
  Description of Artificial Sequence Synthetic polypeptide
  MOD_RES (99)..(99): Any amino acid
EVQLVESGGGLVQAGGSLRLSCAASGRTFSRYAMAWFRHAPGKDREFVAAVSQSGLLTFYADSVKGRFTISRDNAKNTVYLQMNSLKPEDTAVYDCAAXSRFPLVVPVAYENWGQGTQVTVSS

SEQ ID NO: 5 - protein, 123 aa, Artificial Sequence
  Description of Artificial Sequence Synthetic polypeptide
EVQLVESGGGLVQAGGSLRLSCAASGRTFSRYAMAWFRHAPGKDREFVAAVSQSGLLTFYADSVKGRFTISRDNAKNTVYLQMNSLKPEDTAVYDCAADSRFPLVVPVAYENWGQGTQVTVSS

SEQ ID NO: 6 - protein, 116 aa, Artificial Sequence
  Description of Artificial Sequence Synthetic polypeptide
EVQLVESGGGLVQVGGSLRLSCAASGISIRTHAMGWYRQAPGKQRELVATITSVTSGGSLNYADSVKGRFTISRDNAKNTVYLQMNSLKPEDTAVYYCKLLGFDYRGQGTQVTVSS

SEQ ID NO: 7 - protein, 118 aa, Artificial Sequence
  Description of Artificial Sequence Synthetic polypeptide
EVQLVESGGGLVQPGGSLRLSCAASGSIGRFVAMGWYRQAPGKQRELVATITSITSGGRTNYADSVKGRFTISRDNAKNTVYLQMNSLKPEDTAVYYCNVVPYVNDYWGQGTQVTVSS

SEQ ID NO: 8 - protein, 123 aa, Artificial Sequence
  Description of Artificial Sequence Synthetic polypeptide
EVQLVESGGGLVQAGDSLRLSCIASGRTFTMGWFRQAPGKEREFVAAISWSGGRTYYADSVKGRFTISRENAKNTVYLQMNSLKPEDTAVYCCATENLASSGSAYSDDRYNACGQGTQVTVSS

SEQ ID NO: 9 - protein, 124 aa, Artificial Sequence
  Description of Artificial Sequence Synthetic polypeptide
EVQLVESGGEVVQPGGSLRLSCAASGFTFDDRAIGWFRQAPGKEREGVACSANNDNRAFYEDSVKGRFAVSRDNAKNTVYLQMNSLKPEDTAVYYCATRCAAGRVNLYYGMDYWGKGTLVTVSS

SEQ ID NO: 10 - protein, 123 aa, Artificial Sequence
  Description of Artificial Sequence Synthetic polypeptide
EVQLVESGGGLVQPGGSLRLSCAASGFTLGNYAIGWFRQAPGKEREGVSCVDRDGGSTYYLDSVTGRFTTSRDDAENTVYLQMNSLIPDDTAVYYCATRLYGCSGYGRDYADWGQGTQVTVSS

SEQ ID NO: 11 - protein, 118 aa, Artificial Sequence
  Description of Artificial Sequence Synthetic polypeptide
EVQLVESGGGLVQAGGSLRLSCAVSGRTFSTDAFGWFRQAPGKEREFVSAMRWNGSSSYYADLVKGRFTISRDNAKNTVYLLMNSLKPEDTAVYYCTAGKRYGYYDYWGQGTQVTVSS

SEQ ID NO: 12 - protein, 120 aa, Artificial Sequence
  Description of Artificial Sequence Synthetic polypeptide
EVQLVESGGGLVQAGGSLRLSCAASGRTFSNYSMGWFRQAPGKEREFVATISWSGALTHYTDSVKGRFTISRDNAKNTVYLQMNSLKPEDTAVYYCAASDSDYGNKYDYWGQGTQVTVSS

SEQ ID NO: 13 - protein, 119 aa, Artificial Sequence
  Description of Artificial Sequence Synthetic polypeptide
EVQLVESGGGLVQAGGSLRLSCAASGRTVSDMTMGWFRQAPGKERVFVAAISNSGLSTYYQDSVKGRFTISRDTANNTVALQMNSLKPEDTAVYFCAARSGWSGQYDYWGQGTQVTVSS

SEQ ID NO: 14 - protein, 120 aa, Artificial Sequence
  Description of Artificial Sequence Synthetic polypeptide
EVQLVESGGGLVQAGGSLRLSCAASGRIFNNYAMGWFRQAPGKEREFVAGISWSGDSTLYADSVKGRFTTSRDNAKNTVYLQMNSLKPEDTANYYCAEKQGADWAPYDYWGQGTQVTVSS

SEQ ID NO: 15 - protein, 118 aa, Artificial Sequence
  Description of Artificial Sequence Synthetic polypeptide
EVQLVESGGGLVQAGGSLRLSCVASELTFSLYRMGWFRQAPGKEREFVSAMSTSGAGTYYADSVKGRFTISRDNPKNTVYLQMNSLKPEDTAVYYCVAGVRFGVYDYWGQGTQVTVSS

SEQ ID NO: 16 - protein, 129 aa, Artificial Sequence
  Description of Artificial Sequence Synthetic polypeptide
EVQLVESGGGLVQPGGSLRLSCAASGFTLDDYAIGWFRQAPGKEREGVSCISRTDGSTDYADSVKGRFTISRDNAKNTVYLQMNSLKPEDTAVYYCAAGRTYYSGSYYFGLGSDEYDYWGQGTQVTVSS

SEQ ID NO: 17 - protein, 114 aa, Artificial Sequence
  Description of Artificial Sequence Synthetic polypeptide
EVQLVESGGGLVQPGGSLRLSCAASGSIFTINAMAWYRQAPGKQRELVAHLTNSGRTGYADSVKGRFTISSDNAKNTVYLQMNSLKPEDTAVYYCNRLGLHWSWGQGTQVTVSS

SEQ ID NO: 18 - protein, 124 aa, Artificial Sequence
  Description of Artificial Sequence Synthetic polypeptide
EVQLVESGGGLVQAGGSLRLSCAASIGTFSAYHMGWFRQAPGKERELVAAISWSVSSTYYADSVKGRFTISRDNAKRTVSLQMDSLKPEDTAVYYCAARSGERYDYYKAQYEYWGQGTQVTVSS

SEQ ID NO: 19 - protein, 121 aa, Artificial Sequence
  Description of Artificial Sequence Synthetic polypeptide
EVQLVESGGGLVQPGGSLRLSCAAYGSFFSIGTMGWYRQPPGNQRELVAVTYGLGSTNYAESVKGRFTISRDNAKNTVSLQMNSLKPEDTAVYYCYAEIDTDPRSGEWDYWGQGTQVTVSS

SEQ ID NO: 20 - protein, 116 aa, Artificial Sequence
  Description of Artificial Sequence Synthetic polypeptide
EVQLVESGGGLVQPGGSLRLSCLPSTSTSSLRTVGWYRQGPGKQRDLVAIMSAGTTRYADSVKGRFTISLDDAKNTVYLQMNSLKPEDTAVYICNGRPVFSNVDYWGQGTQVTVSS

SEQ ID NO: 21 - protein, 123 aa, Artificial Sequence
  Description of Artificial Sequence Synthetic polypeptide
EVQLVESGGGLVQAGGSLRLSCAASGFTFDDYAIGWFRQAPGKEREGVSCVSRDGGSTYYLDSVKGRFTISSDNAKNTVYLQMNSLKPEDAAVYYCAASRYDCSKYLIDYNYRGQGTQVTVSS

SEQ ID NO: 22 - protein, 125 aa, Artificial Sequence
  Description of Artificial Sequence Synthetic polypeptide
  MOD_RES (49)..(49): Any amino acid
  MOD_RES (52)..(52): Any amino acid
  MOD_RES (57)..(57): Any amino acid
  MOD_RES (104)..(104): Any amino acid
  MOD_RES (116)..(116): Any amino acid
  MOD_RES (119)..(119): Any amino acid
EVQLVKSGGGLVQAGGSLRLSCAASGRRFSTSGMGWFRQAPGREREFVXGIXWNSRXTYYAESVKGRFTISRDNSKNTVYLQMNSLKPEDTAVYYCATNYYGSXWSVNSDDYDYWXQGXQVTVSS

SEQ ID NO: 23 - protein, 126 aa, Artificial Sequence
  Description of Artificial Sequence Synthetic polypeptide
EVQLVESGGGLVQAGGSLRLSCAASGRTFSNYAMGWFRQAPGKEREFVAAITWSGSSTYYADSVKGRFTISRDNAKNTVYLQMNSLKPEDTAVYYCAAAQRGRYYYLDRNVEYDYWGQGTQVTVSS

SEQ ID NO: 24 - protein, 129 aa, Artificial Sequence
  Description of Artificial Sequence Synthetic polypeptide
EVQLVESGGGLVQPGGSLRLSCAASGFTLDDYGIGWFRQAPGKEREGVSCISSSDGSTDYADSVKGRFTISRDNAKNTVYLQMNNLKPEDTAVYYCAAGRTYYSGSYYFGLGSDEYDYWGQGTQVTVSS

SEQ ID NO: 25 - protein, 125 aa, Artificial Sequence
  Description of Artificial Sequence Synthetic polypeptide
EVQLVESGGNLVQAGGSLRLSCAASGFTFDDYVIGWFRQAPGKEREGVSCISSVEGSTYYADSVKGRFTISGDNAKNTVYLQMNSLKPEDTAVYYCAAGTWLDCSGYGSYDMDYWGKGTLVTVSS

SEQ ID NO: 26 - protein, 123 aa, Artificial Sequence
  Description of Artificial Sequence Synthetic polypeptide
EVQLVESGGGLVQAGGSLRLSCAASGFTFDDYVIGWFRQAPGKEREGVSCISSSEGSTYYAESVKGRFTISSDNAKNTVYLQMNSLKPEDTAVYYCAASTWLDFVHGNEYDYRGQGTQVTVSS

SEQ ID NO: 27 - protein, 116 aa, Artificial Sequence
  Description of Artificial Sequence Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 28 - protein, 107 aa, Artificial Sequence
  Description of Artificial Sequence Synthetic polypeptide
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 29 - protein, 130 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVE

SEQ ID NO: 30 - protein, 108 aa, Artificial Sequence
  Description of Artificial Sequence Synthetic polypeptide
LVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 31 - protein, 5 aa, Artificial Sequence
  Description of Artificial Sequence Synthetic peptide
TYAMG

SEQ ID NO: 32 - protein, 4 aa, Artificial Sequence
  Description of Artificial Sequence Synthetic peptide
YDMG

SEQ ID NO: 33 - protein, 5 aa, Artificial Sequence
  Description of Artificial Sequence Synthetic peptide
RYAMA

SEQ ID NO: 34 - protein, 5 aa, Artificial Sequence
  Description of Artificial Sequence Synthetic peptide
THAMG

SEQ ID NO: 35 - protein, 5 aa, Artificial Sequence
  Description of Artificial Sequence Synthetic peptide
FVAMG

SEQ ID NO: 36 - protein, 17 aa, Artificial Sequence
  Description of Artificial Sequence Synthetic peptide
AMISSLSSKSYADTVKG

SEQ ID NO: 37 - protein, 17 aa, Artificial Sequence
  Description of Artificial Sequence Synthetic peptide
AMISSLSSKSYADSVKG

SEQ ID NO: 38 - protein, 17 aa, Artificial Sequence
  Description of Artificial Sequence Synthetic peptide
AINWSGGSTAYADSVKG

SEQ ID NO: 39 - protein, 17 aa, Artificial Sequence
  Description of Artificial Sequence Synthetic peptide
AVSQSGLLTFYADSVKG

SEQ ID NO: 40 - protein, 17 aa, Artificial Sequence
  Description of Artificial Sequence Synthetic peptide
AVSQSGLLTFYADSVKG

SEQ ID NO: 41 - protein, 19 aa, Artificial Sequence
  Description of Artificial Sequence Synthetic peptide
TITSVTSGGSLNYADSVKG

SEQ ID NO: 42 - protein, 19 aa, Artificial Sequence
  Description of Artificial Sequence Synthetic peptide
TITSITSGGRTNYADSVKG

SEQ ID NO: 43 - protein, 16 aa, Artificial Sequence
  Description of Artificial Sequence Synthetic peptide
DLLPYSSSRSLPMGYD

SEQ ID NO: 44 - protein, 17 aa, Artificial Sequence
  Description of Artificial Sequence Synthetic peptide
DLLPYSSTRSLPMGYDY

SEQ ID NO: 45 - protein, 16 aa, Artificial Sequence
  Description of Artificial Sequence Synthetic peptide
KPAKYHFGSGYRDFAE

SEQ ID NO: 46 - protein, 14 aa, Artificial Sequence
  Description of Artificial Sequence Synthetic peptide
  MOD_RES (1)..(1): Any amino acid
XSRFPLVVPVAYEN

SEQ ID NO: 47 - protein, 14 aa, Artificial Sequence
  Description of Artificial Sequence Synthetic peptide
DSRFPLVVPVAYEN

SEQ ID NO: 48 - protein, 5 aa, Artificial Sequence
  Description of Artificial Sequence Synthetic peptide
LGFDY

SEQ ID NO: 49 - protein, 7 aa, Artificial Sequence
  Description of Artificial Sequence Synthetic peptide
VPYVNDY

SEQ ID NO: 50 - protein, 5 aa, Artificial Sequence
  Description of Artificial Sequence Synthetic peptide
DRAIG

SEQ ID NO: 51 - protein, 5 aa, Artificial Sequence
  Description of Artificial Sequence Synthetic peptide
NYAIG

SEQ ID NO: 52 - protein, 5 aa, Artificial Sequence
  Description of Artificial Sequence Synthetic peptide
TDAFG

SEQ ID NO: 53 - protein, 5 aa, Artificial Sequence
  Description of Artificial Sequence Synthetic peptide
NYSMG

SEQ ID NO: 54 - protein, 5 aa, Artificial Sequence
  Description of Artificial Sequence Synthetic peptide
DMTMG

SEQ ID NO: 55 - protein, 5 aa, Artificial Sequence
  Description of Artificial Sequence Synthetic peptide
NYAMG

SEQ ID NO: 56 - protein, 5 aa, Artificial Sequence
  Description of Artificial Sequence Synthetic peptide
LYRMG

SEQ ID NO: 57 - protein, 17 aa, Artificial Sequence
  Description of Artificial Sequence Synthetic peptide
AISWSGGRTYYADSVKG

SEQ ID NO: 58 - protein, 17 aa, Artificial Sequence
  Description of Artificial Sequence Synthetic peptide
CSANNDNRAFYEDSVKG

SEQ ID NO: 59 - protein, 17 aa, Artificial Sequence
  Description of Artificial Sequence Synthetic peptide
CVDRDGGSTYYLDSVTG

SEQ ID NO: 60 - protein, 17 aa, Artificial Sequence
  Description of Artificial Sequence Synthetic peptide
AMRWNGSSSYYADLVKG

SEQ ID NO: 61 - protein, 17 aa, Artificial Sequence
  Description of Artificial Sequence Synthetic peptide
TISWSGALTHYTDSVKG

SEQ ID NO: 62 - protein, 17 aa, Artificial Sequence
  Description of Artificial Sequence Synthetic peptide
AISNSGLSTYYQDSVKG

SEQ ID NO: 63 - protein, 17 aa, Artificial Sequence
  Description of Artificial Sequence Synthetic peptide
GISWSGDSTLYADSVKG

SEQ ID NO: 64 - protein, 17 aa, Artificial Sequence
  Description of Artificial Sequence Synthetic peptide
AMSTSGAGTYYADSVKG

SEQ ID NO: 65 - protein, 17 aa, Artificial Sequence
  Description of Artificial Sequence Synthetic peptide
CISRTDGSTDYADSVKG

SEQ ID NO: 66 - protein, 16 aa, Artificial Sequence
  Description of Artificial Sequence Synthetic peptide
ENLASSGSAYSDDRYN

SEQ ID NO: 67 - protein, 15 aa, Artificial Sequence
  Description of Artificial Sequence Synthetic peptide
RCAAGRVNLYYGMDY

SEQ ID NO: 68 - protein, 14 aa, Artificial Sequence
  Description of Artificial Sequence Synthetic peptide
RLYGCSGYGRDYAD

SEQ ID NO: 69 - protein, 9 aa, Artificial Sequence
  Description of Artificial Sequence Synthetic peptide
GKRYGYYDY

SEQ ID NO: 70 - protein, 11 aa, Artificial Sequence
  Description of Artificial Sequence Synthetic peptide
SDSDYGNKYDY

SEQ ID NO: 71 - protein, 10 aa, Artificial Sequence
  Description of Artificial Sequence Synthetic peptide
RSGWSGQYDY

SEQ ID NO: 72 - protein, 11 aa, Artificial Sequence
  Description of Artificial Sequence Synthetic peptide
KQGADWAPYDY

SEQ ID NO: 73 - protein, 9 aa, Artificial Sequence
  Description of Artificial Sequence Synthetic peptide
GVRFGVYDY

SEQ ID NO: 74 - protein, 5 aa, Artificial Sequence
  Description of Artificial Sequence Synthetic peptide
DYAIG

SEQ ID NO: 75 - protein, 5 aa, Artificial Sequence
  Description of Artificial Sequence Synthetic peptide
INAMA

SEQ ID NO: 76 - protein, 5 aa, Artificial Sequence
  Description of Artificial Sequence Synthetic peptide
AYHMG

SEQ ID NO: 77 - protein, 5 aa, Artificial Sequence
  Description of Artificial Sequence Synthetic peptide
IGTMG

SEQ ID NO: 78 - protein, 5 aa, Artificial Sequence
  Description of Artificial Sequence Synthetic peptide
LRTVG

SEQ ID NO: 79 - protein, 5 aa, Artificial Sequence
  Description of Artificial Sequence Synthetic peptide
DYAIG

SEQ ID NO: 80 - protein, 5 aa, Artificial Sequence
  Description of Artificial Sequence Synthetic peptide
TSGMG

SEQ ID NO: 81 - protein, 5 aa, Artificial Sequence
  Description of Artificial Sequence Synthetic peptide
NYAMG

SEQ ID NO: 82 - protein, 5 aa, Artificial Sequence
  Description of Artificial Sequence Synthetic peptide
DYGIG

SEQ ID NO: 83 - protein, 5 aa, Artificial Sequence
  Description of Artificial Sequence Synthetic peptide
DYVIG

SEQ ID NO: 84 - protein, 16 aa, Artificial Sequence
  Description of Artificial Sequence Synthetic peptide
HLTNSGRTGYADSVKG

SEQ ID NO: 85 - protein, 17 aa, Artificial Sequence
  Description of Artificial Sequence Synthetic peptide
AISWSVSSTYYADSVKG

SEQ ID NO: 86 - protein, 16 aa, Artificial Sequence
  Description of Artificial Sequence Synthetic peptide
VTYGLGSTNYAESVKG

SEQ ID NO: 87 - protein, 15 aa, Artificial Sequence
  Description of Artificial Sequence Synthetic peptide
IMSAGTTRYADSVKG

SEQ ID NO: 88 - protein, 17 aa, Artificial Sequence
  Description of Artificial Sequence Synthetic peptide
CVSRDGGSTYYLDSVKG

SEQ ID NO: 89 - protein, 17 aa, Artificial Sequence
  Description of Artificial Sequence Synthetic peptide
  MOD_RES (3)..(3): Any amino acid
  MOD_RES (8)..(8): Any amino acid
GIXWNSRXTYYAESVKG

SEQ ID NO: 90 - protein, 17 aa, Artificial Sequence
  Description of Artificial Sequence Synthetic peptide
AITWSGSSTYYADSVKG

SEQ ID NO: 91 - protein, 17 aa, Artificial Sequence
  Description of Artificial Sequence Synthetic peptide
CISSSDGSTDYADSVKG

SEQ ID NO: 92 - protein, 17 aa, Artificial Sequence
  Description of Artificial Sequence Synthetic peptide
CISSVEGSTYYADSVKG

SEQ ID NO: 93 - protein, 17 aa, Artificial Sequence
  Description of Artificial Sequence Synthetic peptide
CISSSEGSTYYAESVKG

SEQ ID NO: 94 - protein, 16 aa, Artificial Sequence
  Description of Artificial Sequence Synthetic peptide
GRTYYSGSYYFGLGSD

SEQ ID NO: 95 - protein, 6 aa, Artificial Sequence
  Description of Artificial Sequence Synthetic peptide
LGLHWS

SEQ ID NO: 96 - protein, 15 aa, Artificial Sequence
  Description of Artificial Sequence Synthetic peptide
RSGERYDYYKAQYEY

SEQ ID NO: 97 - protein, 13 aa, Artificial Sequence
  Description of Artificial Sequence Synthetic peptide
EIDTDPRSGEWDY

SEQ ID NO: 98 - protein, 9 aa, Artificial Sequence
  Description of Artificial Sequence Synthetic peptide
RPVFSNVDY

SEQ ID NO: 99 - protein, 14 aa, Artificial Sequence
  Description of Artificial Sequence Synthetic peptide
SRYDCSKYLIDYNY

SEQ ID NO: 100 - protein, 16 aa, Artificial Sequence
  Description of Artificial Sequence Synthetic peptide
  MOD_RES (6)..(6): Any amino acid
NYYGSXWSVNSDDYDY

SEQ ID NO: 101 - protein, 16 aa, Artificial Sequence
  Description of Artificial Sequence Synthetic peptide
AQRGRYYYLDRNVEYD

SEQ ID NO: 102 - protein, 20 aa, Artificial Sequence
  Description of Artificial Sequence Synthetic peptide
GRTYYSGSYYFGLGSDEYDY

SEQ ID NO: 103 - protein, 16 aa, Artificial Sequence
  Description of Artificial Sequence Synthetic peptide
GTWLDCSGYGSYDMDY

SEQ ID NO: 104 - protein, 14 aa, Artificial Sequence
  Description of Artificial Sequence Synthetic peptide
STWLDFVHGNEYDY

SEQ ID NO: 105 - protein, 11 aa, Artificial Sequence
  Description of Artificial Sequence Synthetic peptide
GGQEINSSYGG

SEQ ID NO: 106 - protein, 11 aa, Artificial Sequence
  Description of Artificial Sequence Synthetic peptide
GGSMPNPMVGG

SEQ ID NO: 107 - protein, 11 aa, Artificial Sequence
  Description of Artificial Sequence Synthetic peptide
GGGLQQVLLGG

SEQ ID NO: 108 - protein, 11 aa, Artificial Sequence
  Description of Artificial Sequence Synthetic peptide
GGYAPQRLPGG

SEQ ID NO: 109 - protein, 11 aa, Artificial Sequence
  Description of Artificial Sequence Synthetic peptide
GGAPPHALSGG

SEQ ID NO: 110 - protein, 11 aa, Artificial Sequence
  Description of Artificial Sequence Synthetic peptide
GGVVPTPPYGG

SEQ ID NO: 111 - protein, 133 aa, Artificial Sequence
  Description of Artificial Sequence Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 112 - protein, 116 aa, Artificial Sequence; identical to SEQ ID NO: 27
  Description of Artificial Sequence Synthetic polypeptide

SEQ ID NO: 113 - protein, 107 aa, Artificial Sequence; identical to SEQ ID NO: 28
  Description of Artificial Sequence Synthetic polypeptide

SEQ ID NO: 114 - protein, 17 aa, Artificial Sequence
  Description of Artificial Sequence Synthetic peptide
SSGGGGSGGGGSGGGGS

SEQ ID NO: 115 - protein, 257 aa, Artificial Sequence
  Description of Artificial Sequence Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 116 - protein, 12 aa, Artificial Sequence
  Description of Artificial Sequence Synthetic peptide
YEQDPWGVKWWY

SEQ ID NO: 117 - protein, 12 aa, Artificial Sequence
  Description of Artificial Sequence Synthetic peptide
HLSWLPDVVYAW

SEQ ID NO: 118 - protein, 6 aa, Artificial Sequence
  Description of Artificial Sequence Synthetic peptide
GPLGVR

SEQ ID NO: 119 - protein, 39 aa, Artificial Sequence
  Description of Artificial Sequence Synthetic polypeptide
IARLEEKVKTLKAQNSELASTANMLREQVAQLKQKVMNH

SEQ ID NO: 120 - protein, 39 aa, Artificial Sequence
  Description of Artificial Sequence Synthetic polypeptide
TDTLQAETDQLEDEKSALQTEIANLLKEKEKLEFILAAH

SEQ ID NO: 121 - protein, 31 aa, Artificial Sequence
  Description of Artificial Sequence Synthetic polypeptide
AQLEKELQALEKENAQLEWELQALEKELAQK

SEQ ID NO: 122 - protein, 31 aa, Artificial Sequence
  Description of Artificial Sequence Synthetic polypeptide
AQLKKKLQALKKKNAQLKWKLQALKKKLAQK

SEQ ID NO: 123 - protein, 11 aa, Artificial Sequence
  Description of Artificial Sequence Synthetic peptide
GGSHPRLSAGG

SEQ ID NO: 124 - protein, 11 aa, Artificial Sequence
  Description of Artificial Sequence Synthetic peptide
GGHELSVLLGG

SEQ ID NO: 125 - protein, 11 aa, Artificial Sequence
  Description of Artificial Sequence Synthetic peptide
GGTPRTLPTGG

SEQ ID NO: 126 - protein, 11 aa, Artificial Sequence
  Description of Artificial Sequence Synthetic peptide
GGAPVHSSIGG

SEQ ID NO: 127 - protein, 11 aa, Artificial Sequence
  Description of Artificial Sequence Synthetic peptide
GGTFSNRFIGG

SEQ ID NO: 128 - protein, 11 aa, Artificial Sequence
  Description of Artificial Sequence Synthetic peptide
GGELAPDSPGG

SEQ ID NO: 129 - protein, 7 aa, Artificial Sequence
  Description of Artificial Sequence Synthetic peptide
QEINSSY

SEQ ID NO: 130 - protein, 7 aa, Artificial Sequence
  Description of Artificial Sequence Synthetic peptide
SHPRLSA

SEQ ID NO: 131 - protein, 7 aa, Artificial Sequence
  Description of Artificial Sequence Synthetic peptide
SMPNPMV

SEQ ID NO: 132 - protein, 7 aa, Artificial Sequence
  Description of Artificial Sequence Synthetic peptide
GLQQVLL

SEQ ID NO: 133 - protein, 7 aa, Artificial Sequence
  Description of Artificial Sequence Synthetic peptide
HELSVLL

SEQ ID NO: 134 - protein, 7 aa, Artificial Sequence
  Description of Artificial Sequence Synthetic peptide
YAPQRLP

SEQ ID NO: 135 - protein, 7 aa, Artificial Sequence
  Description of Artificial Sequence Synthetic peptide
TPRTLPT

SEQ ID NO: 136 - protein, 7 aa, Artificial Sequence
  Description of Artificial Sequence Synthetic peptide
APVHSSI

SEQ ID NO: 137 - protein, 7 aa, Artificial Sequence
  Description of Artificial Sequence Synthetic peptide
APPHALS

SEQ ID NO: 138 - protein, 7 aa, Artificial Sequence
  Description of Artificial Sequence Synthetic peptide
TFSNRFI

SEQ ID NO: 139 - protein, 7 aa, Artificial Sequence
  Description of Artificial Sequence Synthetic peptide
VVPTPPY

SEQ ID NO: 140 - protein, 7 aa, Artificial Sequence
  Description of Artificial Sequence Synthetic peptide
ELAPDSP

SEQ ID NO: 141 - protein, 120 aa, Artificial Sequence
  Description of Artificial Sequence Synthetic polypeptide
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 142 - protein, 106 aa, Artificial Sequence
  Description of Artificial Sequence Synthetic polypeptide
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 143 - protein, 118 aa, Artificial Sequence
  Description of Artificial Sequence Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSS

SEQ ID NO: 144 - protein, 135 aa, Artificial Sequence
  Description of Artificial Sequence Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSS

SEQ ID NO: 145 - protein, 6 aa, Artificial Sequence
  Description of Artificial Sequence Synthetic peptide
SGGGGS

SEQ ID NO: 146 - protein, 5 aa, Artificial Sequence
  Description of Artificial Sequence Synthetic peptide
GGGGS

SEQ ID NO: 147 - protein, 4 aa, Artificial Sequence
  Description of Artificial Sequence Synthetic peptide
GGGG

SEQ ID NO: 148 - protein, 20 aa, Artificial Sequence
  Description of Artificial Sequence Synthetic peptide
METDTLLLWVLLLWVPGSTG

SEQ ID NO: 149 - protein, 6 aa, Artificial Sequence
  Description of Artificial Sequence Synthetic 6xHis tag
HHHHHH

SEQ ID NO: 150 - protein, 7 aa, Artificial Sequence
  Description of Artificial Sequence Synthetic peptide
ENLYFQG

SEQ ID NO: 151 - protein, 512 aa, Artificial Sequence
  Description of Artificial Sequence Synthetic polypeptide
METDTLLLWVLLLWVPGSTGHHHHHHENLYFQGEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGQEINSSYGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 152 - protein, 512 aa, Artificial Sequence
  Description of Artificial Sequence Synthetic polypeptide
METDTLLLWVLLLWVPGSTGHHHHHHENLYFQGEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSSGGGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGAPPHALSGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 153 - protein, 4 aa, Artificial Sequence
  Description of Artificial Sequence Synthetic peptide
GSGS

SEQ ID NO: 154 - protein, 4 aa, Artificial Sequence
  Description of Artificial Sequence Synthetic peptide
SGGG

SEQ ID NO: 155 - protein, 7 aa, Artificial Sequence
  Description of Artificial Sequence Synthetic peptide
SGGGGSG

SEQ ID NO: 156 - protein, 33 aa, Artificial Sequence
  Description of Artificial Sequence Synthetic polypeptide
GAAPAAAPAKQEAAAPAPAAKAEAPAAAPAAKA

SEQ ID NO: 157 - protein, 20 aa, Artificial Sequence
  Description of Artificial Sequence Synthetic peptide
  MISC_FEATURE (1)..(20): This sequence may encompass 1-4 ‘Gly Gly Gly Gly Ser’ repeating units
  See specification as filed for detailed description of substitutions and preferred embodiments
GGGGSGGGGSGGGGSGGGGS

SEQ ID NO: 158 - protein, 6 aa, Artificial Sequence
  Description of Artificial Sequence Synthetic peptide
GGGGGG

SEQ ID NO: 159 - protein, 8 aa, Artificial Sequence
  Description of Artificial Sequence Synthetic peptide
GGGGGGGG

SEQ ID NO: 160 - protein, 20 aa, Artificial Sequence
  Description of Artificial Sequence Synthetic peptide
  MISC_FEATURE (1)..(20): This sequence may encompass 1-4 ‘Glu Ala Ala Ala Lys’ repeating units
  See specification as filed for detailed description of substitutions and preferred embodiments
EAAAKEAAAKEAAAKEAAAK